(19) United States
(12) Patent Application Publication
(10) Pub. No.: US 2022/0017895 A1
(43) Pub. Date: Jan. 20, 2022

(54) GRAMC: GENOME-SCALE REPORTER ASSAY METHOD FOR CIS-REGULATORY MODULES

(71) Applicant: Rutgers, The State University of New Jersey, New Brunswick, NJ (US)

(72) Inventor: Jongmin Nam, New Brunswick, NJ (US)

(73) Assignee: Rutgers, The State University of New Jersey, New Brunswick, NJ (US)

(21) Appl. No.: 17/289,841

(22) PCT Filed: Oct. 30, 2019
(86) PCT No.: PCT/US19/58921

§ 371 Date: Apr. 29, 2021

Related U.S. Application Data
(60) Provisional application No. 62/753,608, filed on Oct. 31, 2018.

(51) Int. Cl. C12N 15/10

(52) U.S. Cl. CPC C12N 15/1086

(57) ABSTRACT

Disclosed herein are libraries of reporter nucleic acids for functional regulatory elements as well as methods and kits for constructing and using such libraries. Exemplary libraries, methods, and kits can be used for high-throughput detection, identification, and/or quantitation of functional regulatory elements.

### CROSS REFERENCE TO RELATED APPLICATION

This application claims the benefit of U.S. Provisional Application No. 62/753,608, filed Oct. 31, 2018, which is incorporated by reference herein in its entirety.

### FIELD

This application provides libraries of reporter nucleic acids, for example, functional regulatory elements as well as methods and kits for constructing and using such libraries.

### BACKGROUND

Cis-regulatory modules (CRMs) such as enhancers, promoters, and repressors are functional elements in the genome. It has been estimated that hundreds of thousands of CRMs are scattered across the human genome (Niu, et al. Nucleic acids research 46.11 (2018): 5395-5409; Visel, et al. Nature 461.7261 (2009):199; ENCODE Project Consortium, Nature 489.7414 (2012):57). Because CRMs regulate when, where, and to what level genes are expressed, CRMs are involved in nearly every biological process. Individual CRMs directly interact with multiple transcription factors, and multiple CRMs function in combination to mediate gene regulatory activities (Davidson. The Regulatory Genome, Elsevier (2006); Levine, et al. Cell 157.1 (2014): 13-25; De Laat, et al. Nature 502.7472 (2013): 499). Comprehensive experimental identification of these elements has been a challenge.

The standard reporter assay to identify CRMs is to clone a candidate CRM upstream of a basal promoter and a reporter gene and examine its ability to drive reporter gene expression (Rosenthal, Methods in enzymology 152 (1987): 704-720; Amore, et al. Methods in cell biology 74. (2004): 621-652; Banerji, et al. Cell 27.2 (1981): 299-308). The same reporter construct may monitor how a CRM responds to gene perturbations (Nam, et al. PLoS One 7.4 (2012): e35934.) and to mutations in transcription binding sites (Damle, et al, Developmental biology 357 (2011): 505-517; de-Leon, et al. PNAS USA 107.22 (2010): 10103-10108; Cui, et al, Cell reports 19.2 (2017): 364-374; Emison, et al. Nature 434.7035 (2005): 857; Guerreiro, et al. PNAS USA 110.26 (2013): 10682-10686). However, such conventional one-by-one reporter assays are not suitable for analyzing the millions of potential CRMs contained in the genome (e.g., high-throughput analyses). Some high-throughput assays have been attempted but can suffer from biases.

### SUMMARY

Disclosed herein are methods of constructing a nucleic acid molecule reporter library, as well as nucleic acid molecule reporter libraries produced using the methods disclosed herein. The disclosed genome-scale reporter assay method is effective for both enhancers and promoters as in the case of standard reporter assays. The assay also accommodates long DNA inserts, enabling screening of complete CRMs rather than partial CRMs. Excessive genomic coverage and DNA barcodes increase experimental cost, while insufficient genomic coverage and DNA barcodes results in less reliable data. However, in the libraries and methods disclosed herein, the genomic coverage and the number of DNA barcodes in the library are tunable. Finally, the assay generates reproducible data with comparable or less input materials than currently available methods.

In some embodiments, the methods of constructing a nucleic acid molecule reporter library include isolating a plurality of nucleic acid molecules (e.g., genomic DNA or synthetic DNA) of a selected size range (e.g., a size range of 100-3000 base pairs long, such as about 750-850 base pairs long), ligating the plurality of isolated nucleic acid molecules to at least one linear adapter sequence (such as an adapter including at least two consecutive ribonucleotides flanked by at least one deoxyribonucleotide on a 3′ end, and at least one deoxyribonucleotide on a 5′ end) to form a plurality of circular nucleic acid molecules comprising an insert (an isolated nucleic acid molecule) and an adapter, contacting the plurality of circular nucleic acid molecules with an enzyme under conditions sufficient to produce a plurality of linear nucleic acid molecules, and fusing the plurality of linear nucleic acid molecules to at least one reporter nucleic acid to produce a plurality of reporter constructs, forming the nucleic acid molecule reporter library.

Any nucleic acid molecules can be used, including genomic DNA (such as genomic DNA fragments) or synthetic DNA. In some examples, the nucleic acids are genomic DNA obtained from a cell or population of cells of interest. The genomic DNA can be from any organism of interest, including, but not limited to animals (for example, mammals), plants, bacteria, fungi, or archaea. In some examples, the methods include selecting the size range of the isolated nucleic acid molecules using gel electrophoresis or bead-based size selection. In some examples, the methods include ligating the plurality of isolated nucleic acid molecules to at least one linear adapter sequence using a ligase. In some examples, the ligase includes a DNA ligase, such as a T4 DNA ligase. The linear adapter sequence can include at least two consecutive ribonucleotides flanked by at least one deoxyribonucleotide on a 3′ end and at least one deoxyribonucleotide on a 5′ end (e.g., the nucleic acid of SEQ ID NO: 1 and/or SEQ ID NO: 2). Thus, ligation produces a plurality of circular nucleic acid molecules that include an insert and an adapter.

In some examples, the methods further include contacting the plurality of circular nucleic acid molecules with an exonuclease (e.g., exonuclease I, exonuclease III and/or lambda exonuclease) under conditions sufficient to remove linear nucleic acid molecules from the plurality of circular nucleic acid molecules, prior to linearizing the circular nucleic acids. In some examples, the methods then include contacting the plurality of circular nucleic acid molecules with an endoribonuclease (e.g., an endoribonuclease specific for ribonucleotides within a DNA duplex, such as RNase HII or Uracil-DNA Glycosylase) under conditions sufficient to produce a plurality of linear nucleic acid molecules, each comprising the at least one deoxyribonucleotide on the 3′ end and the at least one deoxyribonucleotide on the 5′ end, flanking the insert. In some examples, the methods include fusing the plurality of linear nucleic acid molecules to at least one reporter nucleic acid (e.g., a nucleic acid encoding a fluorescent protein and/or a nucleic acid that includes a barcode) to produce a plurality of reporter constructs.

In some examples, the methods further include determining genomic coverage of the plurality of linear nucleic acid molecules. For example, determining genomic coverage may include selecting at least one genomic region of interest, amplifying the plurality of linear nucleic acid molecules, and determining the whether the selected genomic region is present in the plurality of linear nucleic acid molecules, the number of copies of the selected genomic region in the plurality of linear nucleic acid molecules, and/or the genomic coverage. In some examples, the genomic coverage is determined by selecting one or more single copy targets for analysis. Exemplary single copy targets include ACTA1, ADM, ADAM12, AXL, CFB, DLXS, Kiss1, NCOA6, Notch2, RPP30, and TOP1. Additional or alternative single copy targets can be selected, depending on the source of the starting material for the library.

In some examples, the methods include fusing the plurality of nucleic acid molecules to a linear vector nucleic acid (e.g., a linear vector nucleic acid that includes a basal promoter). Thus, the methods can be used to produce a plurality of linear vectors comprising nucleic acid molecules.

In some examples, the at least one reporter nucleic acid includes a nucleic acid encoding a fluorescent protein, and fusing the plurality of linear nucleic acid molecules to at least one reporter nucleic acid includes fusing the plurality of linear vectors to a fluorescent reporter nucleic acid. Thus, the methods can be used to produce a plurality of fluorescent reporter constructs. In other examples the at least one reporter nucleic acid includes a nucleic acid encoding a barcode, and fusing the plurality of linear nucleic acid molecules to at least one reporter nucleic acid includes fusing the plurality of reporter linear vectors to a barcode nucleic acid. Thus, the methods can be used to produce a plurality of barcode reporter constructs. In some examples, the at least one reporter nucleic acid includes a nucleic acid encoding a barcode and a nucleic acid encoding a fluorescent protein, and fusing the plurality of linear vectors to at least one reporter nucleic acid includes fusing the plurality of reporter constructs to a barcode nucleic acid and a nucleic acid encoding a fluorescent protein. Thus, the methods can be used to produce a plurality of fluorescent and barcode reporter constructs.

In some examples, the methods further include contacting each of the plurality of linear vectors with a primer nucleic acid that includes a barcode reporter construct. In some examples, the methods then include performing a polymerase chain reaction (PCR). Thus, the methods herein can be used to produce a plurality of amplified vectors that include a barcode reporter construct. In some examples, the methods then include self-ligating the amplified vectors that include a barcode reporter construct to produce circular vectors. Thus, the methods herein can be used to produce a barcode reporter construct. In some examples, the methods herein further include contacting the plurality of circular vectors that include a barcode reporter construct with an exonuclease (e.g., exonuclease I, exonuclease III and/or lambda exonuclease) under conditions sufficient to remove linear nucleic acid molecules from the plurality of circular vectors comprising a barcode reporter construct.

In specific examples of methods of constructing a nucleic acid molecule reporter library the methods include isolating a plurality of nucleic acid molecules of a selected size range; ligating the plurality of isolated nucleic acid molecules to at least one linear adapter sequence using a ligase, wherein the linear adapter sequence includes at least two consecutive ribonucleotides flanked by at least one deoxyribonucleotide on a 3′ end, and at least one deoxyribonucleotide on a 5′ end, thereby producing a plurality of circular nucleic acid molecules that include an insert and an adapter; contacting the plurality of circular nucleic acid molecules with an exonuclease under conditions sufficient to remove linear nucleic acid molecules from the plurality of circular nucleic acid molecules; contacting the plurality of circular nucleic acid molecules with an endoribonuclease under conditions sufficient to produce a plurality of linear nucleic acid molecules each including the at least one deoxyribonucleotide on the 3′ end and the at least one deoxyribonucleotide on the 5′ end, flanking the insert; and fusing the plurality of linear nucleic acid molecules to at least one reporter nucleic acid to produce a plurality of reporter constructs, such as by (a) fusing the plurality of nucleic acid molecules to a linear vector nucleic acid, thereby producing a plurality of linear vectors that include the nucleic acid molecules; (b) contacting each of the plurality of linear vectors that include the nucleic acid molecules with a primer that includes a barcode nucleic acid; and (c) performing a polymerase chain reaction (PCR) and ligation reaction, producing a plurality of circular vectors that include a barcode reporter construct; and contacting the plurality of circular vectors that include the barcode reporter construct with an exonuclease under conditions sufficient to remove linear nucleic acid molecules from the plurality of circular vectors that include a barcode reporter construct. In some examples, the methods further include determining genomic coverage of the inserts prior to fusing the plurality of linear nucleic acids molecules to the at least one reporter nucleic acid.

Further disclosed herein are methods (e.g., high-throughput methods) of detecting functional nucleic acid regulatory elements. In some examples, the methods include transfecting or transforming at least one cell of interest with any of the libraries disclosed herein. Exemplary cells include animal (e.g., mammalian), bacterial, plant, fungal, and archaeal cells. For example, mammalian cells can include cardiomyocytes, neurons, hepatocytes, endothelial cells, embryonic stem cells, organoid-derived cells, organoid-derived cells, and induced stem cells. In some examples, the methods include collecting the at least one cell of interest from at least two subjects, wherein the at least two subjects include at least one subject with a disease or condition and at least one subject without a disease or condition. In some examples, the methods include collecting the at least one cell of interest from at least one subject, wherein the plurality of cells are collected from the subject under different conditions.

In some examples, the methods also include measuring the at least one reporter. For example, some methods can include identifying and/or quantifying the at least one reporter. In some examples, the methods include isolating RNA from the cell of interest to produce isolated RNA. In some examples, identifying the reporter includes reverse transcribing the isolated RNA to produce cDNA, such as using recombinant Moloney murine leukemia virus (rMoMuLV) reverse transcriptase or avian myeloblastosis virus (AMV) reverse transcriptase. In specific examples, an RNA- and DNA-dependent DNA polymerase is also used to reverse transcribe the isolated RNA.

In some examples, the methods then include detecting the cDNA. In some examples, detection includes amplifying the cDNA. For example, where at least one reporter is at least one unique barcode nucleic acid, amplifying the cDNA can include selecting primers specific for nucleotides that include at least one unique nucleic acid barcode, contacting the primers with the cDNA, and performing PCR using the primers and cDNA to produce amplified DNA.

In some examples, the methods further include identifying at least one unique nucleic acid barcode. In some examples, at least one unique nucleic acid barcode is identified through sequencing the amplified DNA. In some examples, the methods also include quantifying at least one unique nucleic acid barcode.

In some examples of the methods herein, the plurality of nucleic acid molecules, for example, the plurality of nucleic acid molecules in a library produced using the methods described herein, include at least 80% of a selected genome of interest. In some examples of the methods herein, the plurality of nucleic acid molecules include at least 80% of the cis-regulatory elements in a selected genome of interest.

Also disclosed herein are kits for constructing a nucleic acid molecule reporter library. In some examples, the kits include at least one of any of the reporter nucleic acids described herein. In some examples, the reporter nucleic acid includes a linear adapter sequence of SEQ ID NO: 1 and/or SEQ ID NO: 2. Exemplary kits can also include at least one ligase, exonuclease, endoribonuclease, and/or polymerase.

Further disclosed herein are kits for high-throughput identification and/or quantitation of functional nucleic acid regulatory elements. In some examples, the kits include any of the libraries disclosed herein, such as libraries that covers at least 80% of a genome of interest. Additional examples of kits include at least one reverse transcriptase and/or PCR primers and a high-fidelity DNA polymerase.

The foregoing and other features of the disclosure will become more apparent from the following detailed description, which proceeds with reference to the accompanying figures.

### BRIEF DESCRIPTION OF THE DRAWINGS

FIGS. 1A-1D: GRAMc library building. FIG. 1A shows an exemplary method of controlling genomic coverage of the library. Size-selected and end-repaired random genomic DNA fragments were circularized by ligation with a fused adapter. Linear DNAs were removed by exonuclease treatment followed by RNaseHII digestion to linearize ligation product and dice adapter-concatemers. Adapter-ligated products were then serially diluted to determine the genomic coverage of each dilution by QPCR. A dilution of intended coverage is assembled using GIBSON ASSEMBLY® with a SCP-GFP cassette and the vector backbone to form barcode-less, linear constructs. FIG. 1B is a schematic showing an exemplary method of controlling barcode numbers of the library. Random 25 bp (N25) barcodes and a core poly-adenylation signal were added to the library of linear constructs by PCR. Barcoded constructs were self-ligated, and linear DNAs were removed by exonucleases I/III. A small fraction of ligates was transformed to determine the scale of transformation. To avoid inflation of colony counts due to cell division, transformants for counting colonies should be immediately plated without rescuing. A desired amount of ligates were transformed to produce a GRAMc library with the intended number of barcodes. Plasmids extracted from liquid media were used for library characterization and reporter assay. Inserts and associated barcodes were identified by Illumina paired-end sequencing. FIG. 1C shows a size distribution of inserts in the human GRAMc library. FIG. 1D shows a cumulative distribution of barcode numbers per insert in the human GRAMc library.

FIGS. 2A-2E show the reproducibility and accuracy of GRAMc. FIG. 2A shows the reproducibility of GRAMc results. The human GRAMc library was tested in two batches of 200M HepG2 cells. CRM activities were double-normalized to the copy numbers of input plasmids and background activity (bg). Inserts that drove reporter expression ≥5×bg in one batch and ≥4.5×bg in another were considered CRMs (“Active”), and the CRM calling was 80% reproducible. Inserts that did not meet the cutoff but were still ≥3×bg in one batch and ≥2.7×bg in another were considered marginally active with a lower reproducibility of 62%. FIG. 2B shows validation of GRAMc results by individual reporter assay. A set of 11 CRMs (“Active”), 5 marginally active inserts and 4 inactive inserts, were tested in 4 batches of individual reporter assays by QPCR. Average activities (solid bar) from 4 batches of individual reporter assays were compared with the GRAMc data (R²=0.83). FIG. 2C shows correlated genomic distributions of CRMs (top) and expressed genes (middle) on chromosome 1. Genomic distribution of the input library is shown at the bottom. Inserts from centromeres were removed. FIG. 2D shows enrichment of CRMs in 2 kb windows with up to 100 kb flanking regions of expressed genes (black dots) and nonexpressed genes (gray dots). The genomic average is shown as a dashed line. The genic region is at position 0 and includes both exons and introns. The area upstream of the genes is on the left half and downstream is shown on the right half. FIG. 2E shows relative enrichment of ENCODE chromatin annotations in CRMs (G5, greater than 5×bg) versus inactive inserts (L1, lower than 1×bg). ENCODE annotations are ordered based on their relative enrichment.

FIGS. 3A-3G show cis-regulatory activity and TFBS motif enrichment in ChromHMM predicted strong enhancers. FIG. 3A shows enrichment of predicted enhancers in CRMs (black bars) versus CRM activities measured by GRAMc (gray bars). Inserts were classified by their averaged activities in two batches of GRAMc data: G5, greater than 5×bg; G3L5, equal or greater than 3×bg and lower than 5×bg; G2L3, equal or greater than 2×bg and lower than 3×bg; G1L2, equal or greater than 1×bg and lower than 2×bg; and L1, lower than 1×bg. FIGS. 3B-3G show relative motif enrichments (log₂ scale) in predicted enhancers with progressively weaker activities versus GRAMc-identified CRMs (G5). Each dot represents a TFBS motif and lines indicate 2-fold differences between the two data sets. The percent proportion of each bin in the predicted enhancers is shown in the upper-left square of each plot.

FIGS. 4A-4E show CRM-driven prediction of gene regulatory programs. FIG. 4A shows abundance and enrichment of TFBS motifs in CRMs. Abundance is the proportion of CRMs (the G5 set) or inactive sets (the L1 set) that contain a given TFBS motif, and the relative enrichment is the ratio of motif enrichments between the G5 set and the L1 set. Vertical lines indicate borders for the relative enrichment of motifs. Several highly enriched and abundant motifs are labeled. FIG. 4B shows comparison of enrichments of predicted TFBS motifs and ENCODE ChIP-seq annotations in the G5 set. FIG. 4C shows two alternative hypotheses on the role of PITX2 or IKZF1 on HepG2-CRMs in other cells (Cell X). FIGS. 4D-4E show testing a hypothesis on the enriched TFBS motifs for non-expressed transcription factors in HepG2 by ectopic expression of human pitx2 (FIG. 4D) and human ikzf1 (FIG. 4E) versus CMV::gfp control. Inserts that belong to the G5 set are shown in red dots (motif+) or in black dots (motif−). Two black diagonal lines indicate 2-fold differences between the perturbed set versus the control set. Inset boxplots show the difference between motif+ versus motif− inserts with P values using a two-sample t-test.

FIGS. 5A-5B show enrichment of repeat elements in GRAMc data. Inserts were classified by their averaged activities in two batches of GRAMc data as in FIGS. 3A-3G. FIG. 5A shows representative families of repeat elements in GRAMc data. Enrichment of repeat elements within genomic regions with differential activities are shown. Genomic regions in the G5 set were considered CRMs. FIG. 5B shows enrichment of three major subfamilies of Alu elements in GRAMc data.

FIGS. 6A-6B show generation of a fused adapter and adapter-ligated inserts. FIG. 6A shows a fused adapter. The fused adapter is prepared by annealing two 5′-phosphorylated oligomers (top, SEQ ID NO: 1; bottom, SEQ ID NO: 2). The fused adapter contains two primer sites, P1 (yellow arrow) and P2 (magenta arrow), for amplification of adapter-ligated genomic inserts. The box indicates two ribonucleotides for an RNase HII cleavage. FIG. 6B shows an exemplary method for preparation of a pure population of adapter-ligated inserts. Ligation of an insert and a fused adapter generated circular DNA that is resistant to exonuclease treatment. All undesired linear DNA was removed by exonuclease I/III. Because circular DNA is difficult to amplify using PCR, circular ligation products were linearized by RNase HII. Linearized adapter-ligated inserts were then ready for PCR amplification with P1 and P2 primers.

FIG. 7 is a schematic diagram showing an exemplary method for preparation of a GRAMc vector for GIBSON ASSEMBLY®. The GRAMc vector is linearized by digestion with AflII and HindIII to increase the efficiency of and reduce the cycles required for amplification. Following digestion, the vector is amplified in two pieces, one containing the SCP-GFP cassette and one containing the vector backbone. Primers NJ96 and NJ95 add the P1 and P2 sites to the vector backbone cassette and the SCP-GFP cassette, respectively, for subsequent GIBSON ASSEMBLY® with adapter ligated inserts. Primers NJ146 and NJ145 contain a sequence of 6 phosporothioated nucleotides at the 5′ end (indicated by S6) to protect the terminal primer sites from degradation during GIBSON ASSEMBLY® and allow for efficient amplification of the pre-barcoded library.

FIG. 8 shows an exemplary method for building paired-end sequencing libraries for Illumina NextSeq500. PCR of the GRAMc library was performed with 2 pairs of primers (P2/nP3 and P1/P4) against adapter sequences flanking the inserts and N25 barcodes, followed by self-ligation, which generates 2 sublibraries with N25s mated to either the 5′ end of inserts (Hs800_14) or the 3′ end of inserts (Hs800_23). Exonuclease treatment ensures survival of only mated circular ligates during subsequent second round amplification of insert::N25 cassettes with the alternate set of primers (P1/P4 for Hs800_23 and P2/nP3 for Hs800_14) to generate 2 sequencing libraries, Hs800_2314 and Hs800_1423. PCR adds PE1 and PE2 sites for Illumina paired-end sequencing. PE1 sites were added using seven out of phase primers per sequencing library to offset the lack of diversity in flanking adapter sequences. Phased primers incorporate ON, 2N, 4N, 6N, 8N, 10N, and 12N random sequences between PE1 sites and respective nP3 or P4 sites. The 14 phased libraries were sequenced on the Illumina NextSeq500 platform.

FIG. 9 shows an exemplary schematic for preparing a GRAMc sequencing library from total RNA. During the first QC step (QC1), removal of contaminated DNA in RNA samples is monitored by measuring GFP DNAs by QPCR. After 12 hours of DNase treatment, if the Ct value for GFP DNA remains ≤30, DNA digestion is continued. The Ct value is observed every 6 hours, and this process is repeated until the Ct value is >30. As a quality control (QC) standard for reverse transcription (RT), 1000 ng of DNaseI/ExoI/ExoIII digested total RNA was used for a standard RT reaction. During the second QC (QC2) step, the genome-scale RT reaction is monitored and supplemented with reagents as needed until the Ct value of GFP cDNA is within 1 cycle of the Ct value in the QC standard.

FIGS. 10A-10F show density over human genome 38 for CRMs, expressed genes, and input. FIGS. 10A-10B show GRAMc CRM density over human genome 38; FIGS. 10C-10D show expressed gene density over human genome 38; and FIGS. 10E-10F show GRAMc input density over human genome 38.

FIG. 11 shows Western blot confirmation of ectopic transcription factor expression. Samples of cells co-transfected with the 80K constructs from the GRAMc library and either with Flag-tagged EGFP (control), or the Flag-tagged transcription factors, PITX2 or IKZF1, were subjected to anti-Flag detection of protein expression. Equivalent sample loading was confirmed with an anti-GAPDH control blot.

FIG. 12 shows an exemplary schematic of GRAMc, including library construction and characterization as well as use of the library in a reporter assay as well as data deconvolution.

FIG. 13 shows an exemplary stepwise synthesis of long random DNA sequences from short random oligomers. De novo synthesis of a large number of long random DNA sequences remains challenging; therefore, a simple method of generating a pool of long random DNA sequences from commercially available short random single stranded DNAs (ssDNAs) is shown. First, 2 μg of ssDNA is phosphorylated using a polynucleotide kinase and subsequently converted into double-strand DNA (dsDNAs) by random hexamers, dNTPs and Klenow enzyme. In parallel, 1 μg of unphosphorylated ssDNA is converted into dsDNA using random hexamers, dNTPs, and Klenow enzyme. Second, a reaction tube is prepared with 200 ng of unphosphorylated dsDNA and T4 DNA ligase in 1× T4 DNA ligase buffer. Unphosphorylated dsDNA ligated to phosphorylated dsDNA. Third, to initiate ligation, 50 ng of phosphorylated dsDNA (or a fraction of unphosphorylated DNA, such as about ¼th) is added to the ligation reaction tube. Because there is an excess amount of unphosphorylated DNA in the reaction, most phosphorylated DNA is ligated to the unphosphorylated DNA. Each molecule of unphosphorylated DNA can accept up to two molecules of phosphorylated DNAs (one molecule on each end). The ligation product includes unphosphorylated 5′-ends. The ligation process is repeated for at least one cycle (e.g., at least about 1, 2, 3, 4, 5, 6, 7, 8, 9, 10, 12, 15, 18, 20, 25, 30, 45, 50, 60, 75, 90, or 100 cycles, or about 1-5, 1-10, 1-15, 1-20, 5-20, 10-25, 25-50, or 50-100 cycles, or about 16 cycles). The cycle number (X) is expected to be ≥2×L/I, where L and I respectively are the desired length of random DNAs and the length of starting oligomers. For example, to synthesize a pool of DNA molecules about 800 bp long with 100 bp-long oligomers, X should be about ≥16. Fourth, nicks in the ligation products are repaired with DNA repair enzymes (NEB PreCR Repair Mix, Cat #M0309S). Fifth, DNAs of a desired length are enriched with gel-based or bead-based size selection. The eluted DNAs are then ready for library construction (e.g., a CRM library), such as a library with at least about 10, 25, 50, 100, 250, 500, 10³, 10⁴, 10⁵, 10⁶, 10⁷, 10⁸, or 10⁹ reporter constructs (e.g., with inserts), such as about 10-100, 100-10³, 10³-10⁴, 10⁴-10⁶, 10⁶-10⁷, 10⁷-10⁸, 10⁸-10⁹, or 10⁶-10⁹ reporter constructs or about 10⁷ reporter constructs, for example, with inserts at least about 50, 100, 200, 300, 400, 500, 750, 800, 900, 1000, 1200, 1500, 2000, 2500, or 3000 base pairs long, such as about 50-3000 or 100-3000 base pairs long, such as about 50-200, 100-200, 100-300, 300-500, 100-1500, 500-1200, 700-1000, or 750-850 base pairs long or about 800 base pairs long. The stepwise synthesis of long, random DNA sequences can also be used in other applications.

FIG. 14 shows the reproducibility of perturbation experiments. Two independent batches of 80,000 randomly selected reporter constructs were compared for each perturbation experiment. All three experiments were highly reproducible (Pearson's r≥0.97).

### SEQUENCE LISTING

The nucleic and amino acid sequences listed in the accompanying sequence listing are shown using standard letter abbreviations for nucleotide bases, and three letter code for amino acids, as defined in 37 C.F.R. 1.822. Only one strand of each nucleic acid sequence is shown, but the complementary strand is understood as included by any reference to the displayed strand. The Sequence Listing is submitted as an ASCII text file, created on Oct. 30, 2019, 30 kb, which is incorporated by reference herein. In the accompanying sequence listing:

SEQ ID NOS: 1 and 2 are exemplary linear adaptor nucleic acid sequences.

SEQ ID NOS: 3-116 are exemplary primer sequences.

SEQ ID NOS: 117-124 are exemplary trimming adaptor sequences.

### DETAILED DESCRIPTION

Unless otherwise noted, technical terms are used according to conventional usage. Definitions of common terms in molecular biology may be found in Benjamin Lewin, Genes VII, published by Oxford University Press, 2000 (ISBN 019879276X); Kendrew et al. (eds.), The Encyclopedia of Molecular Biology, published by Blackwell Publishers, 1994 (ISBN 0632021829); Robert A. Meyers (ed.), Molecular Biology and Biotechnology: a Comprehensive Desk Reference, published by Wiley, John & Sons, Inc., 1995 (ISBN 0471186341); and George P. Rédei, Encyclopedic Dictionary of Genetics, Genomics, and Proteomics, 2nd Edition, 2003 (ISBN: 0-471-26821-6).

The singular forms “a,” “an,” and “the” refer to one or more than one, unless the context clearly dictates otherwise. The term “or” refers to a single element of stated alternative elements or a combination of two or more elements, unless the context clearly indicates otherwise. As used herein, “comprises” means “includes.” Thus, “comprising A or B,” means “including A, B, or A and B,” without excluding additional elements.

It is further to be understood that all base sizes or amino acid sizes, and all molecular weight or molecular mass values, given for nucleic acids or polypeptides are approximate, and are provided for description. Although methods and materials similar or equivalent to those described herein can be used in the practice or testing of the present disclosure, suitable methods and materials are described below. All publications, patent applications, patents, and other references mentioned herein are incorporated by reference in their entirety, as are the GenBank® Accession numbers (for the sequence present on Oct. 31, 2018). In case of conflict, the present specification, including explanations of terms, will control. In addition, the materials, methods, and examples are illustrative only and not intended to be limiting.

To facilitate review of the various embodiments of this disclosure, the following explanations of specific terms are provided.

Adaptor (or adaptor sequence or linker): A single-stranded or double-stranded nucleic acid (e.g., DNA, RNA, or a combination of both) that can be ligated to the ends of other nucleic acid molecules (e.g., DNA and/or RNA). Double stranded adapters can be synthesized to have blunt ends, sticky ends, or a sticky end and a blunt end. In specific examples, the adaptor sequence includes at least one ribonucleotide or at least two consecutive ribonucleotides (e.g., at least about 2, 3, 4, 5, 6, 7, 8, 9, 10, 25, 50, or 100 ribonucleotides, such as about 2-5, 2-10, 2-25, 25-50, or 50-100 ribonucleotides, or about 2 ribonucleotides), for example, flanked by at least one deoxyribonucleotide on the 3′ end and at least one deoxyribonucleotide on the 5′ end (e.g., at least about 1, 2, 5, 10, 15, 16, 17, 18, 19, 20, 21, 22, 23, 24, 25, 26, 27, 28, 29, 30, 31, 32, 33, 34, 35, 40, 45, 50, 100, 250, 500, or 1000 deoxyribonucleotides, or about 5-45, 10-40, 15-35, 20-30, 1-50, 1-100, 1-250, 1-500, or 1-1000 deoxyribonucleotides, or about 21, 28, or 29, or about 15-35 or 20-30 deoxyribonucleotides on the 3′ end and/or the 5′ end). Specific, non-limiting examples of adaptor sequences include SEQ ID NOS: 1 and 2.

Barcode: Any nucleic acid or genetic marker. Barcodes can be random (e.g., for reporter applications, such as high-throughput applications), semi-random, or non-random (e.g., in taxonomic applications, such as unique barcodes that are specific for a taxonomic group for identification of such). In specific examples, the barcode is a random barcode. In some examples, the barcode is from a library of barcodes (e.g., a pre-existing or algorithm-generated barcode library), such as a library of at least 10, 25, 50, 100, 250, 500, 10³, 10⁴, 10⁵, 10⁶, 10⁷, 10⁸, or 10⁹ barcodes, such as about 10-100, 100-10³, 10³-10⁴, 10⁴-10⁶, 10⁶-10⁷, 10⁷-10⁸, 10⁸-10⁹, or 10⁶-10⁹ barcodes or about 10⁷-2×10⁷ barcodes or about 2×10⁷ barcodes. In specific examples, the barcode is from a random library of about 2×10⁷ barcodes. In some examples, the barcode is a short barcode, for example, at least about 5, 10, 15, 20, 25, 30, 35, 40, 45, 50, 75, 100, 250, 500, 1000, 2000, 3000, or 5000 nucleotides long, or about 5-10, 10-20, 15-40, 20-30, 10-50, 10-75, 10-100, 100-250, 250-500, 500-1000, 1000-3000, or 1000-5000 nucleotides long, or about 20, 25, 30, 15-40, or 20-30 nucleotides long.

Complementary: A nucleic acid molecule is said to be complementary to another nucleic acid molecule if the two molecules share a sufficient number of complementary nucleotides (for example, A-T, A-U, or G-C) to form a stable duplex or triplex when the strands bind (hybridize) to each other, for example by forming Watson-Crick, Hoogsteen, or reverse Hoogsteen base pairs. Stable or specific binding occurs when a nucleic acid molecule remains detectably bound to another nucleic acid as a result of base pairing between complementary nucleotides in the nucleic acid molecules under the required conditions.

Conditions sufficient for: Any environment that permits the desired activity, for example, that permits specific binding between two molecules (such as between a nucleic acid and protein or between two nucleic acids) or that permits an enzymatic activity (such as ligase activity or nuclease activity).

Contact: Placement in direct physical association; includes both in solid and liquid form. For example, contacting can occur in vitro or in cells with nucleic acids, proteins, and/or enzymes (e.g., ligases or nucleases).

Detect: To determine if an agent (such as a nucleic acid molecule and/or reporter molecule) is present or absent. In some examples, this can further include identification and/or quantification. For example, use of the disclosed methods and detection probes in particular examples permits determination of presence, amount, and/or identity of a nucleic acid or reporter molecule (such as a reporter nucleic acid).

Hybridization: The ability of complementary single-stranded DNA, RNA, or DNA/RNA hybrids to form a duplex molecule (also referred to as a hybridization complex).

Ligate: Joining together two nucleic acid molecules by a phosphodiester bond between a 3′ hydroxyl group of one nucleic acid molecule and a 5′ phosphate group of a second nucleic acid molecule. An enzyme that catalyzes the formation of the phosphodiester bond between juxtaposed 5′ phosphate and 3′ hydroxyl termini of nucleic acids is referred to as a ligase. Exemplary ligases include DNA ligases (including T4 DNA ligase, T3 DNA ligase, T7 DNA ligase, Taq DNA ligase (e.g., Taq DNA ligase or a high fidelity Taq DNA ligase, such as HiFi Taq DNA ligase)), thermostable DNA ligases (e.g., a thermostable ligase that catalyzes the formation of a phosphodiester bond between the 5′-phosphate and the 3′-hydroxyl of two adjacent DNA strands that are hybridized and accurately paired, with no gap, to a complementary DNA strand, such as 9° N® DNA ligase), and ligases that ligate adjacent, single-stranded DNA splinted by a complementary RNA strand (e.g., SPLINTR® ligase). In some examples, the ligase is sufficient to ligate blunt ends of double-stand nucleic acids (e.g., T4 DNA ligase or T3 DNA ligase). In specific examples, the ligase is T4 DNA ligase.

Nuclease: An enzyme that cleaves a phosphodiester bond. An endonuclease is an enzyme that cleaves an internal phosphodiester bond within a nucleotide chain (in contrast to exonucleases, which cleave a phosphodiester bond at the end of a nucleotide chain). Endonucleases include restriction endonucleases or other site-specific endonucleases, such as endoribonucleases (which cleave RNA at sequence specific sites), for example, RNase HII (e.g., to remove any ribonucleotides) or uracil-DNA glycosylase. Other examples of nucleases include DNase I, Si nuclease, CEL I nuclease, Mung bean nuclease, Ribonuclease A (RNase A), Ribonuclease T1 (RNase T1), Ribonuclease H (RNase H), RNase I, RNase PhyM, RNase U2, RNase CLB, micrococcal nuclease, and apurinic/apyrimidinic endonucleases. Exonucleases include exonuclease I, exonuclease III, lambda exonuclease, exonuclease VII, and Bal 31 nuclease. In particular examples herein, a nuclease is an RNA-specific nuclease, such as RNase HII (e.g., to remove any ribonucleotides) or uracil-DNA glycosylase, or an exonuclease, such as exonuclease I, exonuclease III, or lambda exonuclease.

Regulatory elements: A segment of a nucleic acid molecule which is capable of increasing or decreasing the expression of specific genes. Exemplary regulatory elements include activators, such as promoters (e.g., a region of DNA that initiates transcription of a gene), and enhancers (e.g., a transcription factor or a region of DNA that can interact with other molecules, such as proteins, to increase the likelihood of transcription of a particular gene), or repressors, such as a silencer (e.g., a region of DNA that inhibits transcription of a DNA sequence into RNA when bound to a repressor protein or transcription factor).

Subject: Any multi-cellular vertebrate organism, such as human and non-human mammals (e.g., veterinary subjects).

Vector: A nucleic acid (e.g., DNA or RNA) used as a vehicle to artificially carry foreign genetic material into another cell. Exemplary types of vectors include plasmids, viral vectors, cosmids, and artificial chromosomes. Exemplary elements included in a vector are origin of replications, regulatory elements (e.g., promoters or enhancers), multicloning sites, markers, and/or reporters. In specific examples, a vector can at least include multicloning sites; regulatory elements; for example, promoters (e.g., a basal promoter and/or a synthetic promoter, such as a super core promoter), enhancers, or repressors; and poly(A) tails.

### Methods of Constructing a Nucleic Acid Molecule Reporter Library

Described herein are methods of constructing a nucleic acid molecule reporter library. Thus, methods are provided that allow for a determination of the presence or absence of nucleic acid sequences of interest and/or expression of nucleic acid sequences of interest, such as specific and/or functional sequences within a larger nucleic acid sequence, such as a genome (e.g., an animal or human genome). The methods herein can be used with any nucleic acid sequences of interest, such as functional nucleic acid sequences, for example, nucleic acid sequences that regulate expression of genes (e.g., regulatory elements or modules, such as cis regulatory elements or modules). In some examples, the disclosed methods permit identification or quantitation of the nucleic acids sequences of interest. In some examples, the methods include isolating a plurality of nucleic acid sequences, such as a plurality of nucleic acid sequences that includes nucleic acid sequences of interest, and fusing the plurality of nucleic acid sequences to reporter nucleic acids, producing a plurality of reporter constructs.

In some embodiments, the methods include isolating a plurality of nucleic acid molecules of a selected size range. Any nucleic acid molecules can be used, including genomic DNA (such as genomic DNA fragments) or synthetic DNA. In some examples, the nucleic acids are genomic DNA obtained from a cell or population of cells of interest. Any cell or population of cells can be used, such as animal cells (e.g., mammalian cells), plant cells, bacterial cells, fungal cells, or archaea cells. In some examples, the mammalian cell includes at least one of stem cells, neural cells, cardiovascular cells, hepatic cells, endothelial cells, epithelial cells, oral cells, reproductive cells, endocrine cells, lens cells, fat cells, secretory cells, kidney cells, extracellular matrix cells, contractile cells, immune cells, blood cells, or germ cells. In specific, non-limiting examples, the mammalian cell is at least one of cardiomyocytes, neurons, hepatocytes, endothelial cells (e.g., human umbilical vein endothelial cells, HUVECs, such as in an angiogenesis model), embryonic stem cells, induced pluripotent stem cells, HepG2 cells, LNCaP cells, HeLa cells, HCT116 cells, or K562 cells. In some examples, the plant cell includes at least one of meristematic cells (including meristem derivative cells), parenchyma cells (such as mesophyll cells, transfer cells, or chlorenchyma cells), collenchyma cells, sclerenchyma cells (such as sclerenchyma sclereids or sclerenchyma fibres), tracheids, vessel elements, phloem cells (such as sieve tubes, companion cells, phloem fibres, or phloem sclereids), or epidermal cells (such as a stomatal guard cells). In specific, non-limiting examples, the plant cell is at least one of Arabidopsis, cannabis, maize, rice, barley, wheat, switchgrass, tomato, potato, Chlamydomonas, Hydrodictyon, Spirogyra, and Actebularia. In some examples, the bacterial cell includes at least one of gram-negative or gram-positive bacterial cells, for example, Acidobacteria, Actinobacteria, Aquificae, Bacteroidetes, Caldiserica, Chlamydiae, Chlorobi, Chloroflexi, Chrysiogenetes, Cyanobacteria, Deferribacteres, Deinococcus-Thermus, Dictyoglomi, Escherichia, Elusimicrobia, Fibrobacteres, Firmicutes, Fusobacteria, Gemmatimonadetes, Lentisphaerae, Nitrospira, Planctomycetes, Proteobacteria, Spirochaetes, Synergistetes, Tenericutes, Thermodesulfobacteria, Thermotogae, or Verrucomicrobia cells. In some examples, the fungal cell includes at least one of Trichoderma, Neurospora, Aspergillus, Monascus, Mucor, Saccharomyces, Pichia, or Rhizopus. In some examples, the archaea cell includes at least one of Cenarchaeum, Caldococcus, Ignisphaera, Acidilobus, Acidococcus, Aeropyrum, Desulfurococcus, Ignicoccus, Staphylothermus, Stetteria, Sulfophobococcus, Thermodiscus, Thermosphaera, Geogemma, Hyperthermus, Pyrodictium, Pyrolobus, Nitrosopumilus (candidatus), Acidianus, Metallosphaera, Stygiolobus, Sulfolobus, Sulfurisphaera, Thermofilum, Caldivirga, Pyrobaculum, Thermocladium, Thermoproteus, Vulcanisaeta, Aciduliprofundum, Archaeoglobus, Ferroglobus, Geoglobus, Haladaptatus, Halalkalicoccus, Haloalcalophilium, Haloarcula, Halobacterium, Halobaculum, Halobiforma, Halococcus, Haloferax, Halogeometricum, Halomicrobium, Halopiger, Haloplanus, Haloquadra, Halorhabdus, Halorubrum, Halosarcina, Halosimplex, Haloterrigena, Halovivax, Natrialba, Natrinema, Natronobacterium, Natronococcus, Natronolimnobius, Natronorubrum, Methanoregula (candidatus), Methanocalculus, Methanobacterium, Methanobrevibacter, Methanosphaera, Methanothermobacter, Methanothermus, Methanocaldococcus, Methanotorris, Methanococcus, Methanothermococcus, Methanocorpusculum, Methanoculleus, Methanofollis, Methanogenium, Methanolacinia, Methanomicrobium, Methanoplanus, Methanospirillaceae, Methanospirillum, Methanosaeta, Methanimicrococcus, Methanococcoides, Methanohalobium, Methanohalophilus, Methanolobus, Methanomethylovorans, Methanosalsum, Methanosarcina, Methanopyrus, Palaeococcus, Pyrococcus, Thermococcus, Ferroplasma, Picrophilus, Thermoplasma, Korarchaeota, Nanoarchaeota, or Nanoarchaeum cells.

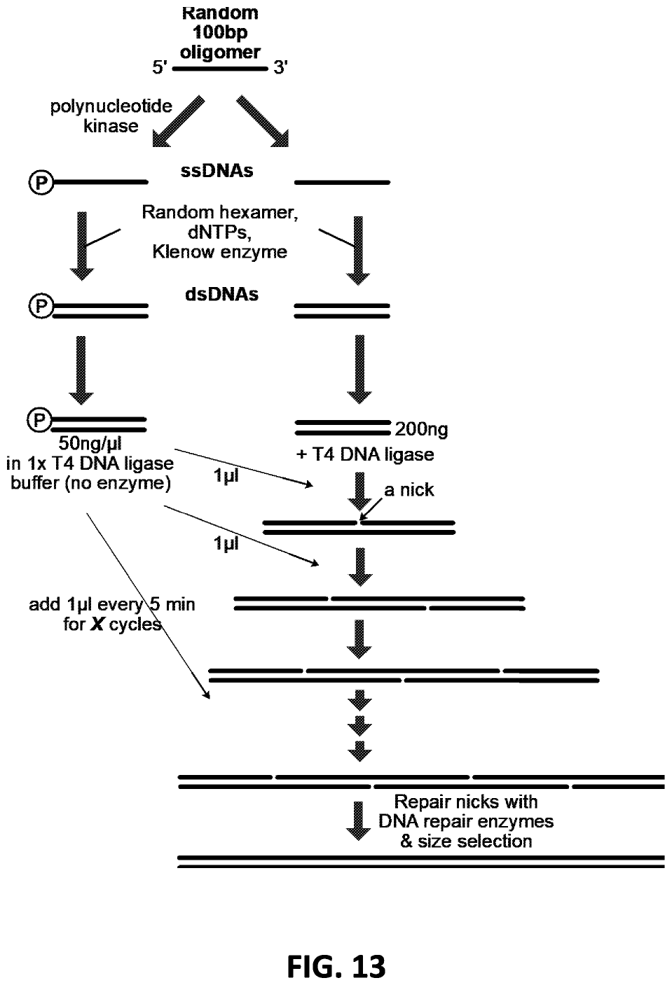

The plurality of nucleic acid molecules of a selected size range can be from any source, for example, a genome or a partial genome from a cell, including chromosomal DNA and mitochondrial DNA. Thus, in some examples, the isolated nucleic acids are isolated from a selected cell type or population of cells types. The DNA (e.g., genomic DNA) is fragmented, for example, by digestion, shearing, sonication, or a combination thereof. In some examples, the nucleic acids are synthetic DNA, such as random double-stranded DNA sequences of a selected length or range of lengths. Any DNA synthesis method can be used to produce synthetic DNA. In specific examples, synthetic DNA (e.g., DNA of a selected size range) can be generated by ligating two or more DNA molecules that are smaller than the DNA of a selected size range (e.g., for DNA in a size selected range of about 750-850 base pairs or about 800 base pairs, the smaller DNA can be at least about 25, 50, 100, 200, 300, or 400 base pairs, or about 25-50, 25-100, 25-200, 25-400, or 100-400 base pairs, or about 100 base pairs). An exemplary method for generating synthetic DNA nucleic acid molecules of a selected size range is shown in FIG. 13.

In some examples, the size range of the nucleic acids that are isolated is at least about 50, 100, 200, 300, 400, 500, 750, 800, 900, 1000, 1200, 1500, 2000, 2500, or 3000 base pairs long, such as about 50-3000 or 100-3000 base pairs long, such as about 50-200, 100-200, 100-300, 300-500, 100-1500, 500-1200, 700-1000, 700-900, or 750-850 base pairs long or about 800 base pairs long. Any method can be used to select a plurality of nucleic acid molecules of a desired size range. In some examples, the plurality of nucleic acid molecules are selected using gel electrophoresis (e.g., using an agarose gel, such as a manually prepared agarose gel or agarose gel cassette, such as using constant voltage or a varying voltage, such as at least a 1%, 1.2%, 1.5%, 2%, 3%, or 5% agarose gel, such as a 1-5%, 1-2%, 2-3%, or 3-5% agarose gel or a 1.2% agarose gel) or bead-based size selection (e.g., solid-phase reversible immobilization, SPRI, such as using paramagnetic beads, for example, paramagnetic beads with a carboxyl coating).

In some examples, the methods include ligating nucleic acid molecules (e.g., the plurality of isolated nucleic acid molecules of the selected size, also referred to herein as “inserts”) to an adapter sequence (e.g., at least one adapter sequence, such as at least one linear adaptor sequence). Any adaptor sequence can be used, such as a linear adapter sequence capable of forming a circular nucleic acid molecule (e.g., a plurality of circular nucleic acid molecules), such as by ligation with the plurality of isolated nucleic acid molecules. In some examples, the adaptor sequence includes ribonucleotides and deoxyribonucleotides. In specific examples, the adaptor sequence includes one ribonucleotide or at least two consecutive ribonucleotides (e.g., at least about 2, 3, 4, 5, 6, 7, 8, 9, 10, 25, 50, or 100 ribonucleotides, such as about 2-5, 2-10, 2-25, 25-50, or 50-100 ribonucleotides, or about 2 ribonucleotides). In some examples, the adaptor sequence includes one ribonucleotide or at least two consecutive ribonucleotides flanked by at least one deoxyribonucleotide at the 3′ end (e.g., at least about 1, 2, 5, 10, 15, 16, 17, 18, 19, 20, 21, 22, 23, 24, 25, 26, 27, 28, 29, 30, 31, 32, 33, 34, 35, 40, 45, 50, 100, 250, 500, or 1000 deoxyribonucleotides, or about 5-45, 10-40, 15-35, 20-30, 1-50, 1-100, 1-250, 1-500, or 1-1000 deoxyribonucleotides, or about 21, 28, or 29, or about 15-35 or 20-30 deoxyribonucleotides at the 3′ end) and at least one deoxyribonucleotide at the 5′ end (e.g., at least about 1, 2, 5, 10, 15, 16, 17, 18, 19, 20, 21, 22, 23, 24, 25, 26, 27, 28, 29, 30, 31, 32, 33, 34, 35, 40, 45, 50, 100, 250, 500, or 1000 deoxyribonucleotides, or about 5-45, 10-40, 15-35, 20-30, 1-50, 1-100, 1-250, 1-500, or 1-1000 deoxyribonucleotides, or about 21, 28, or 29, or about 15-35 or 20-30 deoxyribonucleotides at the 5′ end). In specific examples, the linear adaptor sequence can include the following: CTGCTGAATCACTAGTGAATTATTACCCrUrUCAAGACACTACTCTCCAGCAGT (SEQ ID NO: 1) or CTGCTGGAGAGTAGTGTCTTGrArAGGGTAATAATTCACTAGTGATTCAGCAGT (SEQ ID NO: 2), where the ‘rU’ and ‘rA’ denote ribonucleotides. In particular examples, the adapter is a double-stranded linear adapter prepared by hybridization of the nucleic acids of SEQ ID NOs: 1 and 2.

The plurality of isolated nucleic acid molecules (such as the plurality of inserts) are ligated to the adapter sequence (e.g., at least one adapter sequence, such as at least one linear adaptor sequence, for example, SEQ ID NO: 1 and/or SEQ ID NO: 2) using any ligation method (e.g., ligase-mediated ligation or chemical ligation). In some examples, at least one ligase is used for ligation. Any nucleic acids or adaptor sequence described herein can be used. In some examples, the ligation method is sufficient to form circular nucleic acid molecules (e.g., a plurality of circular nucleic acid molecules) that include the “insert” nucleic acid molecules and the adapter sequence (e.g., a double-stranded adapter including SEQ ID NO: 1 and SEQ ID NO: 2). Thus, in specific examples, the methods can be used to produce a plurality of circular nucleic acid molecules, each with an insert and an adapter sequence. In some examples, a DNA ligase is used. Any ligase (e.g., T4 DNA ligase) sufficient to ligate nucleic acids can be used. Examples of ligases that can be used include DNA ligases (including T4 DNA ligase, T3 DNA ligase, T7 DNA ligase, Taq DNA ligase (e.g., Taq DNA ligase or a high fidelity Taq DNA ligase, such as HiFi Taq DNA ligase), thermostable DNA ligases (e.g., a thermostable ligase that catalyzes the formation of a phosphodiester bond between the 5″-phosphate and the 3″-hydroxyl of two adjacent DNA strands that are hybridized and accurately paired, with no gap, to a complementary DNA strand, such as 9° N® DNA ligase), and ligases that ligate adjacent, single-stranded DNA splinted by a complementary RNA strand (e.g., SPLINTR® ligase). In some examples, the ligase is sufficient to ligate blunt ends of double-stand nucleic acids (e.g., T4 DNA ligase or T3 DNA ligase). In specific examples, the ligase is T4 DNA ligase.

In some embodiments, the methods further include contacting the plurality of circular nucleic acid molecules with at least one enzyme (e.g., at least about 1, 2, 5, or 10 enzymes, or about 1-2, 1-5, or 1-10 enzymes or about 1 or 2 enzymes) specific for removing successive nucleotides from the end of a polynucleotide molecule (e.g., at least one exonuclease, such as at least about 1, 2, 5, or 10 exonucleases, or about 1-2, 1-5, or 1-10 exonucleases, or about 1 or 2 exonucleases) under conditions sufficient to remove linear nucleic acids from circular nucleic acid molecules (e.g., any circular nucleic acid molecules described herein, such as a plurality of circular nucleic acid molecules). In some examples, the at least one exonuclease includes exonuclease I, exonuclease III, and/or lambda exonuclease. In specific examples, the at least one exonuclease is exonuclease I and exonuclease III.

In some embodiments, the methods include contacting the plurality of circular nucleic acid molecules including an insert and adapter sequence with an enzyme specific for separating nucleotides within a polynucleotide chain (e.g., nucleotides other than those at the 5′ or 3′ end, such as an endonuclease) under conditions sufficient to produce linear nucleic acid molecules (e.g., a plurality of linear nucleic acid molecules) from the plurality of circular nucleic acid molecules including an insert and adapter. In some examples, the linear nucleic acid molecules produced each include at least one deoxyribonucleotide on the 5′ end and at least one deoxyribonucleotide on the 3′ end, for example, flanking an insert (e.g., any insert described herein). In some examples, the linear nucleic acid molecules produced include an insert flanked by at least one deoxyribonucleotide on the 5′ end and at least one deoxyribonucleotide on the 3′ end. For example, the at least one deoxyribonucleotide on the 5′ end or the 3′ end can include at least one deoxyribonucleotide, such as about at least about 1, 2, 5, 10, 15, 16, 17, 18, 19, 20, 21, 22, 23, 24, 25, 26, 27, 28, 29, 30, 31, 32, 33, 34, 35, 40, 45, 50, 100, 250, 500, or 1000 deoxyribonucleotides, or about 5-45, 10-40, 15-35, 20-30, 1-50, 1-100, 1-250, 1-500, or 1-1000 deoxyribonucleotides, or about 21, 28, or 29, or about 15-35 or 20-30 deoxyribonucleotides. In specific examples, the enzyme is specific for removing ribonucleotides within a double-stranded nucleic acid (e.g., an endoribonuclease). For example, the enzyme can remove at least one ribonucleotide, such as about at least about 2, 3, 4, 5, 6, 7, 8, 9, 10, 25, 50, or 100 ribonucleotides, such as about 2-5, 2-10, 2-25, 25-50, or 50-100 ribonucleotides, or about 2 ribonucleotides) from a circular nucleic acid (e.g., any of the circular nucleic acid molecules described herein, such as a plurality of circular nucleic acid molecules). In specific examples, the enzyme (e.g., endoribonuclease) can include an RNase HII (e.g., to remove any ribonucleotides) or uracil-DNA glycosylase (e.g., to remove uracil). Linearizing the circular nucleic acids produces a plurality of linear nucleic acid molecules including the insert nucleic acid and at least one deoxyribonucleotide on the 3′ end and at least one deoxyribonucleotide on the 5′ end.

In some embodiments, the methods include fusing the plurality of linear nucleic acid molecules obtained by linearizing the circular nucleic acid including an insert and at least one deoxyribonucleotide on the 3′ end and at least one deoxyribonucleotide on the 5′ end to at least one reporter nucleic acid (e.g., producing a plurality of reporter constructs, such as a nucleic acid molecule reporter library). Any reporter nucleic acid can be used, for example, a fluorescent or barcode reporter nucleic acid, such as nucleic acids encoding a fluorescent protein and/or nucleic acids that include a barcode. In some examples, at least one reporter is a nucleic acid encoding a fluorescent protein. Any fluorescent protein can be encoded, such as a blue, violet, green, yellow, orange, or red fluorescent protein, or a protein with any combination or variation of such fluorescence. In specific examples, at least one reporter nucleic acid is a nucleic acid encoding a green fluorescent protein (GFP). In other examples, at least one reporter nucleic acid is a nucleic acid that includes a barcode (e.g., nucleic acid or genetic marker). Any nucleic acid or genetic marker can be used as a barcode. In some examples, the barcode is a short nucleic acid or genetic marker, for example, a nucleic acid or genetic marker at least about 5, 10, 15, 20, 25, 30, 35, 40, 45, 50, 75, 100, 250, 500, 1000, 2000, 3000, or 5000 nucleotides long, or about 5-10, 10-20, 15-40, 20-30, 10-50, 10-75, 10-100, 100-250, 250-500, 500-1000, 1000-3000, or 1000-5000 nucleotides long, or about 20, 25, 30, 15-40, or 20-30 nucleotides long. In further examples, the reporter includes at least one nucleic acid encoding a fluorescent protein and at least one barcode nucleic acid.

In specific examples, at least one reporter nucleic acid is a barcode nucleic acid. Any nucleic acid barcode can be used; for example, random, semi-random, or non-random barcodes can be used, such as from a barcode library. In specific examples, the barcode is a random barcode. In some examples, the barcode is from a library of barcodes (e.g., a pre-existing or algorithm-generated barcode library), such as a library of at least 10, 25, 50, 100, 250, 500, 10³, 10⁴, 10⁵, 10⁶, 10⁷, 10⁸, or 10⁹ barcodes, such as about 10-100, 100-10³, 10³-10⁴, 10⁴-10⁶, 10⁶-10⁷, 10⁷-10⁸, 10⁸-10⁹, or 10⁶-10⁹ barcodes or about 10⁷-2×10⁷ barcodes or about 2×10⁷ barcodes. In specific examples, the barcode is from a random library of about 2×10⁷ barcodes.

In some embodiments, the methods include fusing the linear nucleic acid molecules including the insert nucleic acid with at least one deoxyribonucleotide on the 3′ end and at least one deoxyribonucleotide on the 5′ end and the reporter to a linear vector nucleic acid to produce a plurality of linear vectors. Any linear vector nucleic acid can be used. For example, a linear vector nucleic acid can include nuclease cleavage sites and transcription or translation regulatory elements (such as promoters, enhancers, repressors, and/or a poly(A) tail). In some examples, the linear vector nucleic acid can include at least one promoter, such as a basal promoter and/or a synthetic promoter. For example, the linear vector nucleic acid can include at least about 1, 2, 3, 4, 5, 6, 8, or 10 promoters, or about 1-4, 5-10, or 1-10 promoters. In some examples, at least one promoter, such as a basal and/or synthetic promoter can include at least one promoter motif, such as at least about 1, 2, 3, 4, 5, 6, 8, or 10 promoter motifs, or about 1-4, 5-10, or 1-10 promoter motifs or about 4 promoter motifs, for example, a synthetic promoter can include TATA box, initiator (Inr), motif ten element (MTE), downstream promoter element (DPE), B recognition element (BRE), E-box, CCAAT box, NRF-1, GABPA, YY1, ACTACAnnTCCC, and/or decamer promoter motifs. In specific examples, at least one promoter is a synthetic promoter that includes TATA box, Inr, MTE, and DPE motifs (e.g., a super core promoter); additional exemplary promoters can be found at Morgan, addgene blog: “Plasmids 101: The Promoter Region—Let's Go!”, 2014, incorporated herein by reference in its entirety.

The linear nucleic acid molecules including the insert nucleic acid with at least one deoxyribonucleotide on the 3′ end and at least one deoxyribonucleotide on the 5′ end can be fused to the linear vector nucleic acid at any time, for example, with, before, or after fusing the linear nucleic acid molecules to at least one reporter nucleic acid. In some examples, the linear vector nucleic acid includes at least one reporter nucleic acid (e.g., at least one reporter nucleic acid encoding a fluorescent protein, such as a green fluorescent protein, or at least one reporter nucleic acid that includes at least one barcode), and, thus, fusing linear nucleic acid molecules to the linear vector nucleic acid includes fusion to at least one reporter nucleic acid. In some examples, the methods include fusing the linear nucleic acid molecules to a linear vector nucleic acid before linear nucleic acid molecules are fused to at least one reporter nucleic acid (e.g., a nucleic acid encoding a fluorescent protein or a nucleic acid that includes a barcode). For example, fusing the plurality of linear nucleic acid molecules to at least one reporter nucleic acid can include fusing the plurality of linear vectors to a reporter nucleic acid encoding a fluorescent protein (e.g., a fluorescent reporter nucleic acid) to produce a plurality of fluorescent reporter constructs. In some examples, fusing the plurality of linear nucleic acid molecules to at least one reporter nucleic acid can include fusing the plurality of linear vectors to a reporter nucleic acid that includes a barcode (e.g., a barcode reporter nucleic acid) to produce a plurality of barcode reporter constructs. In other examples, the linear nucleic acid includes the insert nucleic acid with at least one deoxyribonucleotide on the 3′ end and at least one deoxyribonucleotide on the 5′ end and a reporter nucleic acid before fusing to the linear vector nucleic acid.

The methods include fusing any number of reporter nucleic acids to a plurality of linear nucleic acid molecules or a plurality of linear vectors that include nucleic acid molecules, for example, at least about 1, 2, 3, 4, 5, 10, 15, 20, or 25, or about 1-2, 1-5, 1-10, 10-20, 15-25, or 1-25, or about 2 reporter nucleic acids. In some examples, the methods include fusing plurality of linear nucleic acid molecules or a plurality of linear vectors that include nucleic acid molecules to a fluorescent reporter nucleic acid (e.g., a reporter nucleic acid encoding a GFP) to produce a plurality of fluorescent reporter constructs. In some examples, the methods include fusing a plurality of linear nucleic acid molecules or a plurality of linear vectors that include nucleic acid molecules to a barcode reporter nucleic acid (e.g., a reporter nucleic acid that includes a short barcode, such as a barcode about 25 nucleotides long) to produce a plurality of barcode reporter constructs. In some examples, the methods include fusing a plurality of linear nucleic acid molecules or a plurality of linear vectors that include nucleic acid molecules to a fluorescent reporter nucleic acid and a barcode reporter nucleic acid (e.g., a reporter nucleic acid encoding a GFP and a reporter nucleic acid that includes a short barcode, such as a barcode about 25 nucleotides long) to produce a plurality of fluorescent and barcode reporter constructs. In specific examples, the methods include fusing a plurality of linear vectors that include nucleic acid molecules to a fluorescent reporter nucleic acid and/or a barcode reporter nucleic acid (e.g., a reporter nucleic acid encoding a GFP and/or a reporter nucleic acid that includes a short barcode, such as a barcode about 25 nucleotides long) to produce a plurality of fluorescent and barcode reporter constructs.

In some embodiments, fusing a plurality of linear nucleic acid molecules or a plurality of linear vectors that include nucleic acid molecules to a barcode reporter nucleic acid includes contacting the plurality of linear nucleic acid molecules including the insert nucleic acid with at least one deoxyribonucleotide on the 3′ end and at least one deoxyribonucleotide on the 5′ end or a plurality of linear vectors that include the insert nucleic acid with at least one deoxyribonucleotide on the 3′ end and at least one deoxyribonucleotide on the 5′ end with a primer nucleic acid that includes a barcode reporter nucleic acid (e.g., a reporter nucleic acid that includes a short barcode, such as a barcode about 25 nucleotides long). In some examples, a polymerase chain reaction (PCR) is performed using the plurality of linear nucleic acid molecules or plurality of linear vectors that include the linear nucleic acid molecules and at least one primer nucleic acid that includes a barcode reporter nucleic acid, such as to extend the linear nucleic acid molecules or plurality of linear vectors to produce a plurality of barcode reporter constructs or a plurality of linear vectors that include a barcode reporter constructs. In specific examples, a polymerase chain reaction (PCR) is performed using the plurality of linear vectors that include nucleic acid molecules and primer nucleic acid that includes a barcode reporter nucleic acid to produce a plurality of linear vectors that include a barcode reporter construct.

In some examples, the methods include ligating the ends of the plurality of linear vectors that include the reporter construct (e.g., the fluorescent and/or barcode reporter construct) using a ligase to produce a plurality of circular vectors that include the reporter construct (e.g., the fluorescent and/or barcode reporter construct). In specific examples, the methods include ligating the ends of a plurality of linear vectors that include a barcode reporter construct using a ligase to produce a plurality of circular vectors that include the barcode reporter construct. Any ligase (e.g., a DNA ligase, such as a T4 DNA ligase) described herein can be used. In some examples, the ligase is sufficient to ligate blunt ends of double-stand nucleic acids (e.g., T4 DNA ligase or T3 DNA ligase). In specific examples, the ligase is T4 DNA ligase. In some examples, the methods further include contacting the plurality of circular vectors that include the barcode reporter construct with at least one exonuclease to remove linear nucleic acid molecules from the plurality of circular vectors. Any exonuclease described herein can be used (e.g., exonuclease I, exonuclease III, and/or lambda exonuclease). In specific examples, the at least one exonuclease is exonuclease I and exonuclease III.

In some embodiments, the methods also include determining genomic coverage of the plurality of linear nucleic acid molecules, for example, where the plurality of linear nucleic acid molecules include genomic DNA. The genomic coverage can be determined at any time. In some examples, the genomic coverage is determined prior to fusing the plurality of linear nucleic acid molecules including the inset nucleic acid and at least one deoxyribonucleotide on the 3′ end and at least one deoxyribonucleotide on the 5′ end to the reporter nucleic acid. In specific examples, the coverage can be determined using a plurality of linear nucleic acid molecules (e.g., linear nucleic acid molecules that include nucleic acid molecules and an adapter sequence). Genomic coverage can be determined using any method. In specific examples, genomic coverage is determined by selecting at least one genomic region of interest (e.g., an entire genome or a partial genome), amplifying the plurality of linear nucleic acid molecules (e.g., using PCR, such as quantitative PCR, QPCR), and determining whether the selected genomic region is present in the plurality of linear nucleic acid molecules. In some examples, such as where the linear nucleic acid molecules include nucleic acid molecules and an adapter sequence, the PCR is performed using primers complementary to the adapter sequence (e.g., primers that are complementary to all or part of the adaptor sequence, such as all or part of the adaptor sequence located 5′ to the nucleic acid molecules).

In specific examples of methods of constructing a nucleic acid molecule reporter library, the methods include isolating a plurality of nucleic acid molecules of a selected size range (e.g., at least about 50, 100, 200, 300, 400, 500, 750, 800, 900, 1000, 1200, 1500, 2000, 2500, or 3000 base pairs long, such as about 50-3000 or 100-3000 base pairs long, such as about 50-200, 100-200, 100-300, 300-500, 100-1500, 500-1200, 700-1000, or 750-850 base pairs long or about 800 base pairs long); ligating the plurality of nucleic acid molecules to at least one linear adapter sequence using a ligase (e.g., T4 ligase), wherein the linear adapter sequence includes at least two consecutive ribonucleotides flanked by at least one deoxyribonucleotide on a 3′ end, and at least one deoxyribonucleotide on a 5′ end (e.g., at least about 21, 28, or 29, or about 15-35 or 20-30 deoxyribonucleotides on the 3′ end or the 5′ end), such as SEQ ID NO: 1 or SEQ ID NO: 2, thereby producing a plurality of circular nucleic acid molecules that include an insert and an adapter; contacting the plurality of circular nucleic acid molecules with an exonuclease (e.g., exonuclease I and/or exonuclease III) under conditions sufficient to remove linear nucleic acid molecules from the plurality of circular nucleic acid molecules; contacting the plurality of circular nucleic acid molecules with an endoribonuclease (e.g., RNase HII) under conditions sufficient to produce a plurality of linear nucleic acid molecules each including the at least one deoxyribonucleotide on the 3′ end and the at least one deoxyribonucleotide on the 5′ end, flanking the insert; and fusing the plurality of linear nucleic acid molecules to at least one reporter nucleic acid to produce a plurality of reporter constructs, such as by (a) fusing the plurality of nucleic acid molecules to a linear vector nucleic acid, thereby producing a plurality of linear vectors that include the nucleic acid molecules; (b) contacting each of the plurality of linear vectors that include the nucleic acid molecules with a primer that includes a barcode nucleic acid; and (c) performing a polymerase chain reaction (PCR), producing a plurality of circular vectors that include a barcode reporter construct; and contacting the a plurality of circular vectors that include the barcode reporter construct with an exonuclease (e.g., exonuclease I and/or exonuclease III) under conditions sufficient to remove linear nucleic acid molecules from the plurality of circular vectors that include a barcode reporter construct.

### Compositions and Kits for Constructing a Nucleic Acid Molecule Reporter Library

Contemplated herein are nucleic acid molecule reporter libraries produced using any of the methods described herein. The reporter library can include any number of reporter constructs. In some examples, the number of reporter constructs may depend on the nucleic acid sequence or sequences of interest. For example, where the nucleic acid molecule reporter library includes nucleic acid molecules from a larger sequence, such as a genome (e.g., an animal or human genome, a plant genome, a bacterial genome, a fungal genome, or an archaeal genome), the number of reporter constructs may depend on the size of the larger sequence and/or the level of coverage by the library. In some examples, the number of reporter constructs is at least about 10, 25, 50, 100, 250, 500, 10³, 10⁴, 10⁵, 10⁶, 10⁷, 10⁸, or 10⁹, such as about 10-100, 100-10³, 10³-10⁴, 10⁴-10⁶, 10⁶-10⁷, 10⁷-10⁸, 10⁸-10⁹, or 10⁶-10⁹ or about 10⁷-2×10⁷ or about 2×10⁷ (e.g., 1.91×10⁷).

Contemplated herein are libraries of reporter constructs that include a reporter molecule and nucleic acid molecules (e.g., inserts). The elements of the reporter constructs in nucleic acid molecule reporter libraries produced using the methods herein may also vary depending on the contemplated method of identification and/or quantitation. For example, the libraries produced using the methods herein may be used in vivo or in vitro, and identification and/or quantitation can range from using a visual-based reporter (e.g., a fluorescent reporter, for example, a nucleic acid encoding a blue, violet, green, yellow, orange, or red fluorescent protein, such as for visual and/or spectrometry-based identification and/or quantitation) to a sequence-based reporter (e.g., a barcode reporter, for example, random, semi-random, or non-random barcodes, including nucleic acids or genetic markers at least about 5, 10, 15, 20, 25, 30, 35, 40, 45, 50, 75, 100, 250, 500, 1000, 2000, 3000, or 5000 nucleotides long, or about 5-10, 10-20, 15-40, 20-30, 10-50, 10-75, 10-100, 100-250, 250-500, 500-1000, 1000-3000, or 1000-5000 nucleotides long, or about 20, 25, 30, 15-40, or 20-30 nucleotides long, such as for array-based and/or sequencing-based identification and/or quantitation). Contemplated herein are libraries that include more than one reporter or type of reporter. In some examples, the libraries can include visual- and sequence-based reporters, such as libraries that include fluorescent and barcode reporters. In specific examples, the libraries include reporter constructs with both nucleic acids that encode GFP and that include a short barcode (e.g., a barcode about 25 nucleotides long). The size of the contemplated inserts of the reporter constructs may also vary depending of the contemplated method of identification and/or quantitation. For example, the insert size range is at least about 50, 100, 200, 300, 400, 500, 750, 800, 900, 1000, 1200, 1500, 2000, 2500, or 3000 base pairs long, such as about 50-3000 or 100-3000 base pairs long, such as about 50-200, 100-200, 100-300, 300-500, 100-1500, 500-1200, 700-1000, or 750-850 base pairs long or about 800 base pairs long.

Further contemplated herein are libraries of reporter constructs that include other elements than reporter molecules. For example, the linear adapter sequence of the reporter nucleic acid, or a portion thereof, may be included (e.g., SEQ ID NO: 1 and/or SEQ ID NO: 2 or a portion thereof). For example, the reporter constructs may also include any of the vectors and/or vector elements described herein, such as nuclease cleavage sites and transcription or translation regulatory elements, for example, promoters (e.g., a basal promoter and/or a synthetic promoter, such as a super core promoter), enhancers, repressors, and/or a poly(A) tail.

Also contemplated herein are kits for constructing a nucleic acid molecule reporter library. In some examples, the kits include one or more linear adapters, for example SEQ ID NO: 1 and/or SEQ ID NO: 2. In some examples, the kits include any of the reporter nucleic acids described herein. For example, visual-based nucleic acid reporters (e.g., a fluorescent reporter, for example, a nucleic acid encoding a blue, violet, green, yellow, orange, or red fluorescent protein, such as for visual and/or spectrometry-based identification and/or quantitation) and/or sequence-based reporters (e.g., a barcode reporter, for example, random, semi-random, or non-random barcodes, including nucleic acids or genetic markers at least about 5, 10, 15, 20, 25, 30, 35, 40, 45, 50, 75, 100, 250, 500, 1000, 2000, 3000, or 5000 nucleotides long, or about 5-10, 10-20, 15-40, 20-30, 10-50, 10-75, 10-100, 100-250, 250-500, 500-1000, 1000-3000, or 1000-5000 nucleotides long, or about 20, 25, 30, 15-40, or 20-30 nucleotides long, such as for array-based and/or sequencing-based identification and/or quantitation) can be included. More than one reporter or type of reporter is contemplated. For example, the kits can include visual- and sequence-based reporters, such as fluorescent and barcode reporters. In specific examples, the kits include nucleic acids reporters that both encode GFP and include a short barcode (e.g., a barcode about 25 nucleotides long).

Further contemplated herein are kits with reporter constructs that include other elements than reporter molecules. For example, the linear adapter sequence of the reporter nucleic acid may be included (e.g., SEQ ID NO: 1 and/or SEQ ID NO: 2). The kits may also include any of the vectors and/or vector elements described herein, such as nuclease cleavage sites and transcription or translation regulatory elements, for example, promoters (e.g., a basal promoter and/or a synthetic promoter, such as a super core promoter), enhancers, repressors, and/or a poly(A) tail. Also contemplated are any of the enzymes for performing the methods described herein. For example, the kit can include at least one ligase, such as DNA ligases (including T4 DNA ligase, T3 DNA ligase, T7 DNA ligase, Taq DNA ligase (e.g., Taq DNA ligase or a high fidelity Taq DNA ligase, such as HiFi Taq DNA ligase), thermostable DNA ligases (e.g., a thermostable ligase that catalyzes the formation of a phosphodiester bond between the 5′-phosphate and the 3′-hydroxyl of two adjacent DNA strands that are hybridized and accurately paired, with no gap, to a complementary DNA strand, such as 9° N® DNA ligase), and ligases that ligate adjacent, single-stranded DNA splinted by a complementary RNA strand (e.g., SPLINTR® ligase); at least one exonuclease, such as at least about 1, 2, 5, or 10 exonucleases, or about 1-2, 1-5, or 1-10 exonucleases, or about 1 or 2 exonucleases (e.g., exonuclease I, exonuclease III, and/or lambda exonuclease); endoribonuclease (e.g., RNase HII or uracil-DNA glycosylase), and/or polymerase, including any polymerase suitable for PCR (e.g., a high-fidelity polymerase).

### Methods of Detecting Functional Nucleic Acid Regulatory Elements and Kits Therefor

The disclosed libraries can be used for a variety of purposes, including identifying cis-regulatory elements in a genome of interest. In some examples, the disclosed libraries can be used to directly measure functional differences in CRMs from different individuals of the same species. The disclosed libraries and methods can directly measure functional consequences of sequence variations in cell-based approaches (e.g., cardiomyocytes, neurons, hepatocytes). In other examples, the disclosed libraries and methods can be used to identify biomarker CRMs, such as CRMs that mediate cellular toxicity of a drug, CRMs that maintain pathological state of cells, and/or CRMs that maintain healthy cellular states

For example, the disclosed libraries and methods can identify CRMs that respond to cellular toxicity of a drug. A collection of biomarker CRMs that detect multiple different cellular toxicity effects can be generated and this collection of biomarkers can be used to test drugs' toxicity in one screening. The disclosed libraries and methods can also identify CRMs that are specific to pathological cell state in patient-derived cells (e.g., iPSC-derived cardiomyopathic cells). The disclosed libraries and methods further be used to identify CRMs that are specific to healthy cell states in control cells (e.g., iPSC-derived control cardiomyocytes). Furthermore, by pooling all three types of biomarker CRMs, one can screen drugs that can turn pathological cell state into normal state without causing cytotoxic effect in a single screening.

In another embodiment, the disclosed libraries and methods can screen artificial CRMs that possess any desired activity. These CRMs can include a strong driver for selection markers in any cell type (e.g., drivers for precisely controlling gene expression (e.g., enzymes) in engineered cells (bacteria, fungi, plants, archaea, and mammalian cells).

In other embodiments, the disclosed libraries and methods can screen enriched motifs for non-expressed transcription factors in a host cell type, such as to detect gene regulatory interactions, for example, in various cell types (e.g., mutually exclusive cell types, for example, formed from stem cells, such as embryonic stem cells or induced stem cells). Exemplary applications include tissue engineering, for example, to generate a particular cell type. For example, one cell type can be suppressed and another cell type can be promoted (e.g., for applications where one cell type can turn into another cell type, for example, where a desired cell type or cell type of interest can turn into an undesired cell type or cell type that is not of interest).

Disclosed herein are methods of detecting functional nucleic acid regulatory elements (for example, CRMS, such as promoters, enhancers, and/or repressors). In some examples, the methods can include transfecting at least one cell of interest with a nucleic acid molecule reporter library disclosed herein. In some examples, the methods include selecting a cell of interest. Any cell of interest can be used and/or selected, such as animal cells (e.g., mammalian cells), plant cells, fungal cells, bacterial cells, or archaea cells. In some examples, the mammalian cell includes at least one of stem cells, neural cells, cardiovascular cells, hepatic cells, endothelial cells, epithelial cells, oral cells, reproductive cells, endocrine cells, lens cells, fat cells, secretory cells, kidney cells, extracellular matrix cells, contractile cells, immune cells, blood cells, or germ cells. In specific, non-limiting examples, the mammalian cell is at least one of cardiomyocytes, neurons, hepatocytes, endothelial cells (e.g., human umbilical vein endothelial cells, HUVECs, such as in an angiogenesis model), embryonic stem cells, induced pluripotent stem cells, HepG2 cells, LNCaP cells, HeLa cells, HCT116 cells, or K562 cells. In some examples, the plant cell includes at least one of meristematic cells (including meristem derivative cells), parenchyma cells (such as mesophyll cells, transfer cells, or chlorenchyma cells), collenchyma cells, sclerenchyma cells (such as sclerenchyma sclereids or sclerenchyma fibres), tracheids, vessel elements, phloem cells (such as sieve tubes, companion cells, phloem fibres, or phloem sclereids), or epidermal cells (such as a stomatal guard cells). In specific, non-limiting examples, the plant cell is at least one of Arabidopsis, cannabis, maize, rice, barley, wheat, switchgrass, tomato, potato, Chlamydomonas, Hydrodictyon, Spirogyra, and Actebularia. In some examples, the bacterial cell includes at least one of gram-negative or gram-positive bacterial cells, for example, Acidobacteria, Actinobacteria, Aquificae, Bacteroidetes, Caldiserica, Chlamydiae, Chlorobi, Chloroflexi, Chrysiogenetes, Cyanobacteria, Deferribacteres, Deinococcus-Thermus, Dictyoglomi, Elusimicrobia, Escherichia, Fibrobacteres, Firmicutes, Fusobacteria, Gemmatimonadetes, Lentisphaerae, Nitrospira, Planctomycetes, Proteobacteria, Spirochaetes, Synergistetes, Tenericutes, Thermodesulfobacteria, Thermotogae, or Verrucomicrobia cells. In some examples, the fungal cell includes at least one of Trichoderma, Neurospora, Aspergillus, Monascus, Mucor, Saccharomyces, Pichia, or Rhizopus. In some examples, the archaea cell includes at least one Cenarchaeum, Caldococcus, Ignisphaera, Acidilobus, Acidococcus, Aeropyrum, Desulfurococcus, Ignicoccus, Staphylothermus, Stetteria, Sulfophobococcus, Thermodiscus, Thermosphaera, Geogemma, Hyperthermus, Pyrodictium, Pyrolobus, Nitrosopumilus (candidatus), Acidianus, Metallosphaera, Stygiolobus, Sulfolobus, Sulfurisphaera, Thermofilum, Caldivirga, Pyrobaculum, Thermocladium, Thermoproteus, Vulcanisaeta, Aciduliprofundum, Archaeoglobus, Ferroglobus, Geoglobus, Haladaptatus, Halalkalicoccus, Haloalcalophilium, Haloarcula, Halobacterium, Halobaculum, Halobiforma, Halococcus, Haloferax, Halogeometricum, Halomicrobium, Halopiger, Haloplanus, Haloquadra, Halorhabdus, Halorubrum, Halosarcina, Halosimplex, Haloterrigena, Halovivax, Natrialba, Natrinema, Natronobacterium, Natronococcus, Natronolimnobius, Natronorubrum, Methanoregula (candidatus), Methanocalculus, Methanobacterium, Methanobrevibacter, Methanosphaera, Methanothermobacter, Methanothermus, Methanocaldococcus, Methanotorris, Methanococcus, Methanothermococcus, Methanocorpusculum, Methanoculleus, Methanofollis, Methanogenium, Methanolacinia, Methanomicrobium, Methanoplanus, Methanospirillaceae, Methanospirillum, Methanosaeta, Methanimicrococcus, Methanococcoides, Methanohalobium, Methanohalophilus, Methanolobus, Methanomethylovorans, Methanosalsum, Methanosarcina, Methanopyrus, Palaeococcus, Pyrococcus, Thermococcus, Ferroplasma, Picrophilus, Thermoplasma, Korarchaeota, Nanoarchaeota, or Nanoarchaeum cells.

In some examples, the methods include collecting at least one cell of interest (e.g., from at least one subject). In some examples, the cells are collected from at least two subjects, such as at least one subject with a disease or condition and at least one subject without a disease or condition. In other examples, the cells are collected from cells or subjects under different conditions (e.g., before or after administration of a reagent or protocol, such as a drug or treatment protocol). Any of the libraries described herein can be used. The methods can also include measuring the at least one reporter. In some embodiments, the methods also include identifying and/or quantifying at least one reporter. In particular embodiments, identifying and/or quantifying at least one reporter indicates presence of one or more CRMs linked to the reporter. The CRM can be further characterized, for example by isolating the nucleic acid linked to the reporter and sequencing the nucleic acid. The isolated nucleic acid can further be tested to identify the CRM included in the nucleic acid.

In some embodiments, the methods include isolating RNA from the cell of interest that has been transfected with the nucleic acid reporter library, thereby producing isolated RNA. Any method can be used to isolate RNA, including extraction and precipitation methods (e.g., Tan et al. Journal of biomedicine & biotechnology (2009): 574398-574398, incorporated herein by reference in its entirety). In some examples, additional steps can be included, such as to enhance the purity of the isolated RNA. Any additional RNA isolation steps can be included, such as contacting the RNA with enzymes specific for DNA, for example, DNases (e.g., DNase I) and/or exonucleases (e.g., exonuclease I and/or exonuclease III).

In some embodiments, identifying the reporter includes synthesizing cDNA. In some examples, synthesizing cDNA includes reverse transcribing isolated RNA (e.g., RNA isolated using any of the methods described herein), thereby producing cDNA. Any method of reverse transcription can be used. In some examples, the methods include contacting the isolated RNA with at least one reverse transcriptase. Any reverse transcriptase can be used. In some examples, the recombinant Moloney murine leukemia virus (rMoMuLV) reverse transcriptase and/or avian myeloblastosis virus (AMV) reverse transcriptase can be used. Any additional cDNA synthesis steps can be included. In specific examples, additional cDNA synthesis steps include further contacting the RNA and the at least one reverse transcriptase with an RNA- and DNA-dependent DNA polymerase. In some examples, additional cDNA synthesis steps include adding RNase (e.g., an RNase specific for single-strand RNA, such as RNase I_{f}).

In some embodiments, the methods include detecting and/or identifying cDNA (e.g., cDNA synthesized using any of the methods described herein). Any method of detecting and/or identifying cDNA can be used (e.g., sequencing-, microarray-, and/or PCR-based methods, such as Next Generation sequencing methods, microarray and hybridization, and/or quantitative PCR). In some examples, the cDNA includes at least one unique barcode reporter. In some examples, detecting cDNA includes amplifying cDNA (e.g., using PCR, such as high-fidelity PCR, for example, by contacting the cDNA with a high-fidelity polymerase and/or at least one primer, such as a pair of universal primers), such as the barcode reporter cDNA (e.g., barcode reporter cDNA). In specific examples, the amplifying the cDNA includes selecting primers specific for nucleotides that include at least one unique nucleic acid barcode (e.g., at least one primer, such as a pair of primers, for example a pair of universal primers). In some examples, the primers include a pair of universal primers that amplifies the pool of barcodes in the cDNA. In some examples, amplifying the cDNA further includes contacting the primers with the cDNA and performing PCR (e.g., using the primers and the cDNA). Thus, in some examples, the methods can be used to produce amplified DNA (e.g., cDNA), such as amplified barcode DNA. In some examples, the methods include identifying the cDNA, such as by identifying a reporter (e.g., a nucleic acid barcode). In some examples, the methods include identifying a nucleic acid barcode using sequencing-, microarray-, and/or PCR-based methods, such as Next Generation sequencing, microarray and hybridization, and/or quantitative PCR. In specific examples, the cDNA is identified by sequencing a nucleic acid barcode (e.g., using Next Generation sequencing). Exemplary methods can further include a quantitation step (e.g., quantifying the at least one unique nucleic acid barcode).

In some examples, the methods described herein are high-throughput methods. In some examples, the plurality of nucleic acid molecules in the libraries described herein cover at least about 10%, 20%, 30%, 40%, 50%, 60%, 70%, 75%, 80%, 85%, 90%, 91%, 92%, 93%, 94%, 95%, 98%, or 100%, or about 10-20%, 20-40%, 25-50%, 50-75%, 75-85%, 80-90%, 85-90%, 85-100%, or 90-100%, or about 93%, 93.4%, or 94% of a selected genome of interest (e.g., an animal or human genome). In other examples, the plurality of nucleic acids in the library provides greater than 1× coverage of a genome (for example, 1×, 1.5×, 2×, 2.5×, 3×, 3.5×, 4×, 4.5×, 5×, 8×, 10×, or greater coverage). In some examples, the plurality of nucleic acid molecules include at least about 10%, 20%, 30%, 40%, 50%, 60%, 70%, 75%, 80%, 85%, 90%, 91%, 92%, 93%, 94%, 95%, 98%, or 100%, or about 10-20%, 20-40%, 25-50%, 50-75%, 75-85%, 80-90%, 85-90%, 85-100%, or 90-100%, or about 85%, 90%, or 95% of the cis regulatory elements in a selected genome of interest.

Further contemplated herein are kits for detecting functional nucleic acid regulatory elements. In some examples, the kits can be used for identification and/or quantitation of functional nucleic acid regulatory elements. In some examples, the kits can be used for high-throughput detection, identification, and/or quantitation of functional nucleic acid regulatory elements. In some examples, the kits can include any nucleic acid reporter library described herein. In some examples, the library covers at least about 10%, 20%, 30%, 40%, 50%, 60%, 70%, 75%, 80%, 85%, 90%, 91%, 92%, 93%, 94%, 95%, 98%, or 100%, or about 10-20%, 20-40%, 25-50%, 50-75%, 75-85%, 80-90%, 85-90%, 85-100%, or 90-100%, or about 93%, 93.4%, or 94% of a selected genome of interest (e.g., an animal or human genome). In some examples, the library includes at least about 10%, 20%, 30%, 40%, 50%, 60%, 70%, 75%, 80%, 85%, 90%, 91%, 92%, 93%, 94%, 95%, 98%, or 100%, or about 10-20%, 20-40%, 25-50%, 50-75%, 75-85%, 80-90%, 85-90%, 85-100%, or 90-100%, or about 85%, 90%, or 95% of the cis regulatory elements in a selected genome of interest (e.g., an animal or human genome).

In some examples, the kits further include at least one reverse transcriptase (e.g., recombinant Moloney murine leukemia virus (rMoMuLV) reverse transcriptase, avian myeloblastosis virus (AMV) reverse transcriptase). Additional cDNA synthesis elements can be included, such as an RNA- and DNA-dependent DNA polymerase and/or RNase (e.g., an RNase specific for single-strand RNA, such as RNase I_{f}). In some examples, the kits include elements for amplification (e.g., of cDNA, such as cDNA that includes at least one unique barcode), such as by PCR. In specific examples, the kits include PCR primers and a DNA polymerase (e.g., a high-fidelity DNA polymerase).

### EXAMPLES

The following examples are provided to illustrate certain particular features and/or embodiments. These examples should not be construed to limit the disclosure to the particular features or embodiments described. These examples describe a Genome-scale Reporter Assay Method for cis-regulatory modules (CRMs). GRAMc can reliably measure the cis-regulatory activity of nearly 90% of the human genome in 200 million HepG2 cells with randomly fragmented inserts of about 800 bp. A library of reporter constructs was generated that covers the human genome about 4 times (4× coverage) with ≥15 M randomly fragmented inserts of about 800 bp.

### Example 1

This example describes methods and materials used in Examples 1-7.

### GRAMc Library Construction

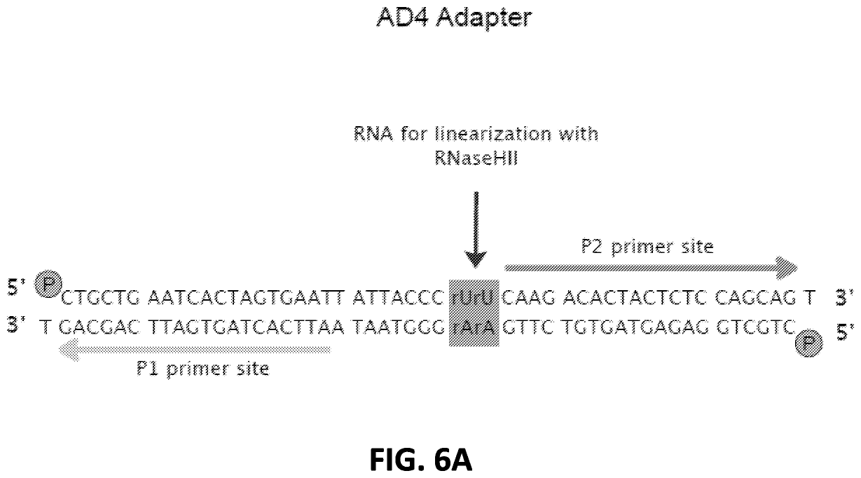

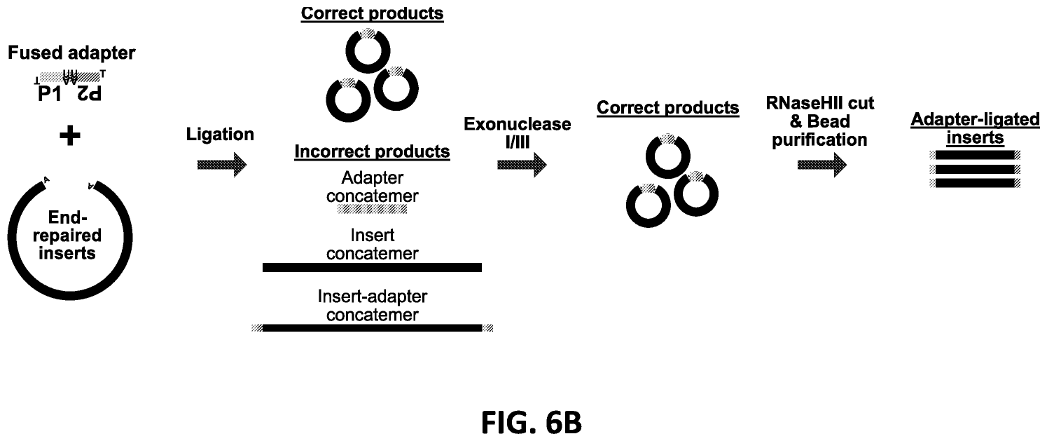

Fused adapter preparation: GRAMc preparation includes a custom-designed fused adapter to minimize the formation of unwanted concatenates (FIG. 6). Two complementary hybrid oligomers were synthesized by Integrated DNA Technologies (IDT): p-AD4_F (5′-/p/CTGCTGAATCACTAGTGAATTATTACCCrUrUCAAGACACTACTCTCCAGCAGT-3; SEQ ID NO: 1) and p-AD4_R (5′-/p/CTGCTGGAGAGTAGTGTCTTGrArAGGGTAATAATTCACTAGTGATTCAGCAGT-3; SEQ ID NO: 2)). Ribonucleotide sites are labeled “rU” and “rA.” A fused adapter was prepared by diluting p-AD4_F and p-AD4_R to 4 pmol/μL in 1× T4 DNA ligase buffer (NEB® B0202S) followed by annealing at 95° C. for 2 min, then decreasing the temperature for 160 cycles at a rate of −0.5° C./20 s cycle. Annealed adapters were aliquoted into 3 μl volume and maintained at −80° C. until use.

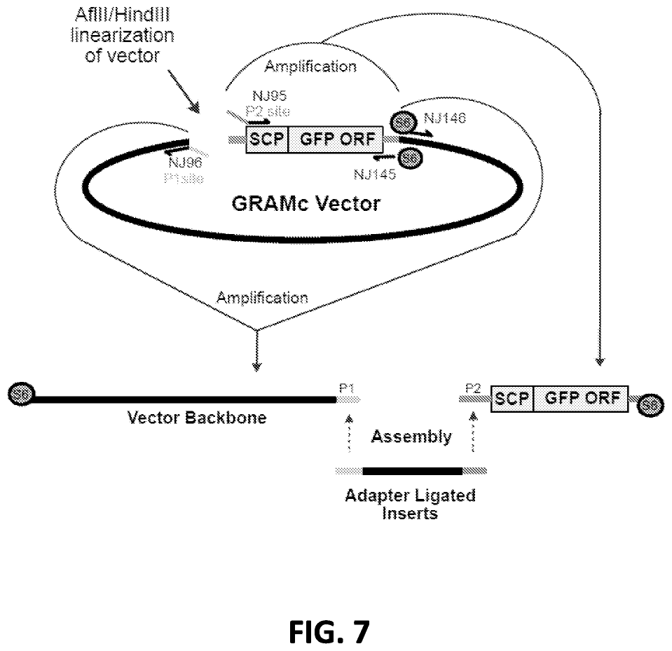

GRAMc vector preparation: The GRAMc vector was constructed by replacing sea urchin nodal basal promoter with the Super Core Promoter 1 (SCP) (Juven-Gershon, et al. Developmental biology 339.2 (2010): 225-229) upstream of the GFP ORF in an existing vector (Nam, et al, PLoS One 7.4 (2012): e35934) based on pGEM-T Easy vector (PROMEGA®). The GFP ORF is from pGREEN LANTERN® (GIBCO BRL®) (Arnone, et al. Development 124.22 (1997): 4649-4659). The vector was linearized by AflII/HindIII overnight digestion and amplified in 10 cycle of PCR as two separate cassettes from 20 ng of linearized template (FIG. 7). The SCP-GFP cassette was amplified in a 50 μL Q5® High-Fidelity DNA Polymerase reaction (NEB® M0491) using primers NJ-95 and NJ-145 and the vector backbone with NJ-146 and NJ-96 using an annealing temperature of 62° C. and a 2 min extension. A sequence of six phosporothioated bases at the 5′ end of the NJ145 and NJ146 prevents loss of primer sites during subsequent GIBSON ASSEMBLY®.

Preparation of genomic inserts: Twenty micrograms of NG16408 genomic DNA (Coriell Institute) was randomly fragmented in 200 μL of water with a QSONICA® Q125 at 20% amperage with 3 cycles of 15 s pulses/10 s rest. DNA was column cleaned using a Zymo-25 column (Zymo Research) and size selected for about 800 bp fragments on a 1.2% agarose gel. A portion of the gel-purified gDNA was size confirmed on a 2% agarose E-gel (THERMOFISHER® G501802). The remaining purified fragments were repaired in a 25 μL PreCR reaction (NEB® M0309) containing 1× THERMOPOL® Buffer, 100 μM dNTPs, 1×NAD+, and 0.5 μL of PreCR enzyme for 30 minutes at 37° C. PreCR-treated fragments were column purified using a Zymo-6 column and treated with the End Repair/dA Tailing Module (NEB® E7370) in a 32.5 μL reaction, followed by a 41 μL reaction of the TA Ligation Module (NEB E7370) with a 10:1 adapter to insert molar ratio of the annealed AD4 fused adapter. Unligated adapters and genomic inserts were removed with 20 U each of exonuclease I (NEB M0293) and exonuclease III (NEB® M0206) in a 50 μL reaction supplemented to 1× with CutSmart buffer. Ligates were column cleaned (Zymo-6), then linearized with 15 U of RNase HII (NEB® M0288) in a 30 μL reaction in 1× THERMOPOL® buffer for 90 minutes at 37° C. RNase HII also cuts concatemers of AD4 adapters into about 60 bp units, which can be removed in subsequent magnetic bead purification. Linearized inserts were purified using 20 μL of AXYGEN® magnetic beads (AXYGEN®), supplemented to a final concentration of 17% PEG 8000 and 10 mM MgCl₂, followed by 3 washes with 70% ethanol and elution in 30 μL of water.

Stepwise synthesis of long random DNA sequences from short random oligomers: Because de novo synthesis of a large number of long random DNA sequences remains challenging, in some examples, a pool of long random DNA sequences were generated from commercially available short random single stranded DNAs (ssDNAs; FIG. 13). First, 2 μg of ssDNA was phosphorylated using a polynucleotide kinase and subsequently converted into double-strand DNA (dsDNAs) by random hexamers, dNTPs, and Klenow enzyme. In parallel, 1 μg of unphosphorylated ssDNA was converted into dsDNA using random hexamers, dNTPs, and Klenow enzyme. Second, a reaction tube was prepared with 200 ng of unphosphorylated dsDNA and T4 DNA ligase in 1× T4 DNA ligase buffer. Unphosphorylated dsDNA was ligated to phosphorylated dsDNA. Third, to initiate ligation, 50 ng of phosphorylated dsDNA (or a fraction of unphosphorylated DNA, such as about ¼th) was added to the ligation reaction tube. Because there was an excess amount of unphosphorylated DNA in the reaction, most phosphorylated DNA was ligated to the unphosphorylated DNA. Each molecule of unphosphorylated DNA can accept up to two molecules of phosphorylated DNAs (one molecule on each end). The ligation product includes unphosphorylated 5′-ends. The ligation process was repeated for at least one cycle (e.g., at least about 1, 2, 3, 4, 5, 6, 7, 8, 9, 10, 12, 15, 18, 20, 25, 30, 45, 50, 60, 75, 90, or 100 cycles, or about 1-5, 1-10, 1-15, 1-20, 5-20, 10-25, 25-50, or 50-100 cycles, or about 16 cycles). The cycle number (X) is expected to be ≥2×L/I, where L and I respectively are the desired length of random DNA generated and the length of starting nucleic acid. For example, to synthesize a pool of DNA molecules about 800 bp long with 100 bp-long nucleic acids, X should be about ≥16. Fourth, nicks in the ligation products were repaired with DNA repair enzymes (NEB® PreCR Repair Mix, Cat #M0309S). Fifth, DNA molecules of a desired length were enriched with gel-based or bead-based size selection. The eluted DNA was then ready for GRAMc library building or other applications. Using this method, we have generated a GRAMc library that contains approximately 1M random DNA sequences about 800 bp long.

Genomic coverage estimation: To determine the amount of adapter-ligated inserts that represent 1× genomic coverage, dilutions of 0.5 ng/μl, 0.25 ng/μl, 0.1 ng/μl, 0.05 ng/μl, and 0.025 ng/μL of insert were prepared. Each dilution was amplified with two adapter-specific primers, NJ-213 and NJ-214, with annealing at 61° C. and a 1 minute extension as determined by a cycle test. A Q5® High-Fidelity DNA Polymerase kit (NEB® M0491) was used. Amplicons were AXYGEN® cleaned. Eight nanograms per well of each amplified dilution and of NG16408 stock DNA was used for QPCR against the following single copy targets: ACTA1, ADM, ADAM12, AXL, CFB, DLXS, Kiss1, NCOA6, Notch2, RPP30, and TOP1. For each dilution sample, targets with a dCT>5 compared to stock genomic DNA were counted as absent.

The Poisson probability (P) of a genomic region being present in the library is given as P=1−(1−p)XN, in which p=(insert size)/(genome size), N=the number of partitions of the genome for the given insert size, and X=the intended genomic coverage. The proportion of targets present as identified by QPCR were compared to the value of P. Based on this model, the P was about 0.6 for a sample with about 1× genomic coverage. The 0.1 ng/μL dilution tested positive for 6 of the 11 targets or a proportion of 0.545, representing between 0.5× and 1× coverage. Thus, 0.2 ng of inserts were determined to represent about 1× genomic coverage. Equimolar amounts of independently amplified replicates were mixed to obtain a pool of inserts at 5× genomic coverage.

Insert cloning and N25 barcoding of the GRAMc library: Thirty nanograms of 5× genomic inserts were cloned into the two-pieces of linearized GRAMc vector, SCP-GFP, and the backbone cassettes with a 1:1:1 molar ratio in a 16 μL NEBUILDER® HiFi Assembly reaction (NEB® E2621) for 20 minutes at 50° C. Assembled linear DNA was column purified and eluted in 20 μL water. To prepare the assembled library for barcoding, 4 replicates of 8 ng of the purified assembly were amplified in 9 cycles of PCR, as determined by a cycle test, with primers NJ-101 and NJ-126 using an annealing temperature of 62° C. and a 5 minute extension time. The replicates were combined and column-cleaned.

To add N25 barcodes downstream of the GFP ORF, 150 ng of the library was used for a single cycle of PCR with NJ-127, which contains random 25 bp barcode sequences, core Poly(A) signal (Nag, et al. RNA 12.8 (2006): 1534-1544) and 5′ biotinylation, in a 50 μL Q5 High-Fidelity DNA Polymerase reaction with an annealing temperature of 60° C. for 40 seconds and an extension time of 15 minutes. NJ-126 was used as a competitor in the PCR to reduce the potential for template switching by occupying and extending the opposing strand. Primers were removed by AXYGEN® bead purification using 50 μL of beads and 20 μL water elution, as has been described. The barcoded library was isolated using 20 μL of DYNABEADS® MyOne C1 beads (INVITROGEN® 65001) with bead preparation, binding, and washing according to the manufacturer's protocol.

Following isolation, C1 beads were washed in 20 μL of water then resuspended in 50 of water. Half of the barcoded library was amplified in 24×20 μL replicate Q5® High-Fidelity DNA Polymerase reactions for 9 cycles, as determined by a cycle test, with NJ-128 and NJ-129, 61° C. annealing, and a 5 minute extension. Replicates were combined and AXYGEN®-bead cleaned, then gel purified (Zymo Research) with an additional AXYGEN® bead cleaning.

The barcoded GRAMc library was then self-ligated. To reduce intermolecular ligation, 125 ng of the barcoded library was ligated in 600 μL of 1× T4 ligase buffer (NEB® B0202) with 14,000 U of high-concentration T4 DNA Ligase (NEB® M0202T) for 4 hrs at 20° C. Ligation products were supplemented with 67 μL of lambda exonuclease buffer and 30 U each of exonuclease I (NEB® M0293) and lambda exonuclease (NEB® M0262S) for 1 hour at 37° C., then spiked with 1 μL of Proteinase K (THERMOFISHER®) for 15 minutes at 37° C. Proteinase K treatment reduces viscosity of the ligation mix and increases DNA yield by nearly two fold. The library was purified with 25 μL of magnetic beads (AXYGEN®) supplemented to a final concentration of 15% PEG 8000 and 10 mM MgCl₂, followed by 4 washes with 70% ethanol and elution in 6.5 μL of water. The product of this process is a pure population of circularized GRAMc library.

Transformation and size estimation of the GRAMc library: To determine the scale of electroporation, 1 μl of ligation product was electroporated into 25 μL of ELECTROMAX® DH10B® competent cells (THERMOFISHER® 18290015). Transformants were resuspended into 1 ml of pre-warmed SOC media immediately, and 1/500th of the transformants were used for 10-fold serial dilution and plating without recovery to estimate the number of colonies for the entire pool. The scale of transformation to reach the target colony number is determined based on this test. Electroporation of 4-10 ng of ligation products generates about 40 M colonies.

To generate a full GRAMc library with a colony target of 200 M, duplicate electroporation steps were performed using 30 ng of library ligates (12 ng/μL) per each of 2×25 of ELECTROMAX® DH10B® competent cells. Each replicate was resuspended into 1 ml of SOC media immediately following electroporation, and then the replicates were combined. To estimate the size of the GRAMc library, 1/2000 of the transformants were used for a 10-fold serial dilution and plating without recovery. The remaining transformants were immediately used to inoculate 180 ml of LB, to which 100 μg/ml ampicillin was added following a 20 minute recovery followed by overnight culturing. The plasmid library was prepared using the ZYMOPURE® II Plasmid Maxiprep Kit (Zymo Research). Hereafter, this library is referred to as the Hs800_GRAMc library.

As a quality control step, twelve colonies from the plate were picked, and plasmids were extracted to check the insert sizes and barcodes using Sanger sequencing. Plasmids from each colony should contain an insert (about 800 bp) and a barcode. Where the ligation product includes high barcode diversity, the barcode sequences identified from colonies should not be present in the final library. Example sequences of GRAMc vector and oligomers used are available in Table 3.

TABLE 3
Example primer and adaptor trimming sequences

| Primer/ | | |
|---|---|---|
| Adaptor | Name | Sequence |
| NJ-95 | Gibson_SCP1_amp | CTGCTGGAGAGTAGTGTCTTGtACTTATATAAGG |
| | 1 | GGGTGGG (SEQ ID NO: 3) |
| NJ-96 | EcoP151_Plr_lin | CTGCTGAATCACTAGTGAATTCGCGG (SEQ ID |
| | | NO: 4) |
| NJ-101 | 3PofN25 merShort | GGCGCGCCGCTGAGGGAGT (SEQ ID NO: 5) |
| NJ-126 | NT_del_F | AATTCGCCCTATAGTGAGTCGTA (SEQ ID NO: 6) |
| NJ-127 | bN25_polyA_R-1; | /5Biosg/TACAGTCCGACGATCCAGCAG(N:2525252 |
| | “primer R” | 5)(N)(N)(N)(N)(N)(N)(N)(N)(N)(N)(N)(N)(N)(N)(N)(N) |
| | | (N)(N)(N)(N)(N)(N)(N)(N)GGCGCGCCGCTGAGGGA |
| | | GTCTAGAG (SEQ ID NO: 7) |
| NJ-128 | pN25_polyA_R-2 | /5Phos/CACAAACCACAACTAGAATGCAGTGAAA |
| | | AAAATGCTTTATTTGTTTACAGTCCGACGATCCA |
| | | GCAG (SEQ ID NO: 8) |
| NJ-129 | pNT_del_F | pAATTCGCCCTATAGTGAGTCGTA (SEQ ID NO: |
| | | 9) |
| NJ-132 | GRAMc_Ion- | CCATCTCATCCCTGCGTGTCTCCGACTCAGTTCG |
| | A_IX7_P4s | TGATTCGATTACAGTCCGACGATCCAGCAG (SEQ |
| | | ID NO: 10) |
| NJ-133 | GRAMc_Ion- | CCATCTCATCCCTGCGTGTCTCCGACTCAGTTCC |
| | A_IX8_P4s | GATAACGATTACAGTCCGACGATCCAGCAG |
| | | (SEQ ID NO: 11) |
| NJ-134 | GRAMc_Ion- | CCATCTCATCCCTGCGTGTCTCCGACTCAGTGAG |
| | A_IX9_P4s | CGGAACGATTACAGTCCGACGATCCAGCAG |
| | | (SEQ ID NO: 12) |
| NJ-141 | pGRAMc_nP3 short | /5Phos/TAGACTCCCTCAGCGGC (SEQ ID NO: 13) |
| NJ-142 | pGRAMc_P4 short | /5Phos/TACAGTCCGAcgatcCAGCAG (SEQ ID NO: |
| | | 14) |
| NJ-145 | S_3PofN25 merShort | G*G*C*G*C*G*CCGCTGAGGGAGT (* = |
| | | Phosphorothioated bonds)(SEQ ID NO: 15) |
| NJ-146 | S_NT_del_F | A*A*T*T*C*G*CCCTATAGTGAGTCGTA (* = |
| | | Phosphorothioated bonds)(SEQ ID NO: 16) |
| NJ-179 | CRSP_F_T7_ | TTAATACGACTCACTATAGGTCGTAGTTATCTAC |
| | backbone | ACGACGGTTTTAGAGCTAGAAATAG (SEQ ID |
| | | NO: 17) |
| NJ-180 | CRSP_F_T7_GFP | TTAATACGACTCACTATAGGCGCGCTGAAGTCA |
| | | AGTTCGAGTTTTAGAGCTAGAAATAG (SEQ ID |
| | | NO: 18) |
| NJ-183 | CRSP_R | AAAAGCACCGACTCGGTGCC (SEQ ID NO: 19) |
| NJ-197 | GRAMc_Ion- | CCATCTCATCCCTGCGTGTCTCCGACTCAGCTAA |
| | A_IX1_P4s | GGTAACGATTACAGTCCGACGATCCAGCAG |
| | | (SEQ ID NO: 20) |
| NJ-198 | GRAMc_Ion- | CCATCTCATCCCTGCGTGTCTCCGACTCAGTAAG |
| | A_IX2_P4s | GAGAACGATTACAGTCCGACGATCCAGCAG |
| | | (SEQ ID NO: 21) |
| NJ-200 | GRAMc_Ion- | CCATCTCATCCCTGCGTGTCTCCGACTCAGAAGA |
| | A_IX3_P4s | GGATTCGATTACAGTCCGACGATCCAGCAG |
| | | (SEQ ID NO: 22) |
| NJ-208 | pGRAMc_P1s_NoT | /5Phos/ATTCACTAGTGATTCAGCAG (SEQ ID NO: |
| | | 23) |
| NJ-209 | pGRAMc_P2s_NoT | /5Phos/GACACTACTCTCCAGCAG (SEQ ID NO: 24) |
| NJ-213 | Gibson_P1-T | GCGAATTCACTAGTGATTCAGCAGT (SEQ ID NO: |
| | | 25) |
| NJ-214 | Gibson_iNBP-T | CAAGACACTACTCTCCAGCAGT (SEQ ID NO: 26) |
| NJ-268 | Hs-Top1_QF | ACTTCGTGTGGAGCACATCA (SEQ ID NO: 27) |
| NJ-269 | Hs-Top1_QR | CGTTTCTCAACAGGGACCTT (SEQ ID NO: 28) |
| NJ-270 | Hs-ACTA1_QF | ATGGTCGGTATGGGTCAGAA (SEQ ID NO: 29) |
| NJ-271 | Hs-ACTA1_QR | TCTCCATGTCATCCCAGTTG (SEQ ID NO: 30) |
| NJ-276 | Hs-AXL_QF2 | CTGTCAGACGATGGGATGG (SEQ ID NO: 31) |
| NJ-277 | Hs-AXL_QR2 | TAAGGGGTGTGAGGATGGAG (SEQ ID NO: 32) |
| NJ-278 | Hs-DLX5_QF | TACACAAGTGCAGCCAGCTC (SEQ ID NO: 33) |
| NJ-279 | Hs-DLX5_QR | GAGTAAGAGAGAGCAGCCCATC (SEQ ID NO: 34) |
| NJ-280 | Hs-NOTCH2_QF | AAATGCCTCACAGGCTTCAC (SEQ ID NO: 35) |
| NJ-281 | Hs-NOTCH2_QR | CACTGGCACTGGTAGGAACC (SEQ ID NO: 36) |
| NJ-282 | Hs-RPP30_QF | CTGCTTCCAGGAGACCTGAC (SEQ ID NO: 37) |
| NJ-283 | Hs-RPP30_QR | TTTGTGGTGATTTCCCCCTA (SEQ ID NO: 38) |
| NJ-284 | Hs-ADM_QF | GGTCGGACTCTGGTGTCTTC (SEQ ID NO: 39) |
| NJ-285 | Hs-ADM_QR | CTTGCGCGACTATTCCTTGT (SEQ ID NO: 40) |
| NJ-286 | Hs-CFB_QF | CAAGCAGACAAGCAAAGCAA (SEQ ID NO: 41) |
| NJ-287 | Hs-CFB_QR | GATAAAGGGCATCAGGCAGA (SEQ ID NO: 42) |
| NJ-288 | Hs-Kiss1_QF | ACCTGCCGAACTACAACTGG (SEQ ID NO: 43) |
| NJ-289 | Hs-Kiss1_QR | TTTGGGGTCTGAAGTTCACTG (SEQ ID NO: 44) |
| NJ-292 | Hs-NCOA6_QF | TGGCTTCTCAGCAGGACAG (SEQ ID NO: 45) |
| NJ-293 | Hs-NCOA6_QR | TGCTGGACATTTTGATTTGC (SEQ ID NO: 46) |
| NJ-294 | Hs-ADAM12_QF | CAGTTGCAGCAGGAAGGACT (SEQ ID NO: 47) |
| NJ-295 | Hs-ADAM12_QR | TCCACAAATCTGTTCCCACA (SEQ ID NO: 48) |
| NJ-364 | PE2_GRAMC_P4s | CAAGCAGAAGACGGCATACGAGATGTGACTGGA |
| | | GTTCAGACGTGTGCTCTTCCGATCTACAGTCCGA |
| | | CGATCCAGCAG (SEQ ID NO: 49) |
| NJ-399 | PE2_GRAMC_P3s | CAAGCAGAAGACGGCATACGAGATGTGACTGGA |
| | | GTTCAGACGTGTGCTCTTCCGATCTTAGACTCCC |
| | | TCAGCGGC (SEQ ID NO: 50) |
| NJ-400 | PE1_GRAMC_P3s | AATGATACGGCGACCACCGAGATCTACACTCTT |
| | | TCCCTACACGACGCTCTTCCGATCTTAGACTCCC |
| | | TCAGCGGC (SEQ ID NO: 51) |
| NJ-401 | PE2_GRAMC_P2s | CAAGCAGAAGACGGCATACGAGATGTGACTGGA |
| | | GTTCAGACGTGTGCTCTTCCGATCTACACTACTC |
| | | TCCAGCAG (SEQ ID NO: 52) |
| NJ-402 | PE1_GRAMC_P4s | AATGATACGGCGACCACCGAGATCTACACTCTT |
| | | TCCCTACACGACGCTCTTCCGATCTTACAGTCCG |
| | | ACGATCCAGCAG (SEQ ID NO: 53) |
| NJ-403 | PE2_GRAMC_P1s | CAAGCAGAAGACGGCATACGAGATGTGACTGGA |
| | | GTTCAGACGTGTGCTCTTCCGATCTTTCACTAGT |
| | | GATTCAGCAG (SEQ ID NO: 54) |
| NJ-404 | EGFPC1_QF1 | AAGGGCATCGACTTCAAGGA (SEQ ID NO: 55) |
| NJ-405 | EGFPC1_QR1 | GGCGGATCTTGAAGTTCACC (SEQ ID NO: 56) |
| NJ-443 | GRAMc_GFP_QF2 | GCCCTGTCTAAAGATCCCAA (SEQ ID NO: 57) |
| NJ 444 | GRAMc_GFP_QR2 | CTTGTACAGCTCGTCCATGC (SEQ ID NO: 58) |
| NJ-489 | GRAMc_RT oligo | TACAGTCCGACGATCC (SEQ ID NO: 59) |
| NJ-497 | PE1_adapter | AATGATACGGCGACCACCGAGATCTACACTCTT |
| | | TCCCTACACGACGCTCTTCCGATCT (SEQ ID NO: |
| | | 60) |
| NJ-498 | PE1s_GRAMc_2N | TACACGACGCTCTTCCGATCTNNTACAGTCCGAC |
| | _P4s | GATCCAGCAG (SEQ ID NO: 61) |
| NJ-499 | PE1s_GRAMc_4N | TACACGACGCTCTTCCGATC TACAGTCCG |
| | _P4s | ACGATCCAGCAG (SEQ ID NO: 62) |
| NJ-500 | PE1s_GRAMc_6N | TACACGACGCTCTTCCGATC TACAGT |
| | _P4s | CCGACGATCCAGCAG (SEQ ID NO: 63) |
| NJ-501 | PE1s_GRAMc_8N | TACACGACGCTCTTCCGATC TACA |
| | _P4s | GTCCGACGATCCAGCAG (SEQ ID NO: 64) |
| NJ-502 | PE1s_GRAMc_10 | TACACGACGCTCTTCCGATC TA |
| | N_P4s | CAGTCCGACGATCCAGCAG (SEQ ID NO: 65) |
| NJ-503 | PE1s_GRAMc_12 | TACACGACGCTCTTCCGATC |
| | N_P4s | TACAGTCCGACGATCCAGCAG (SEQ ID NO: 66) |
| NJ-504 | PE1s_GRAMc_2N | TACACGACGCTCTTCCGATCTNNTAGACTCCCTC |
| | _nP3s | AGCGGC (SEQ ID NO: 67) |
| NJ-505 | PE1s_GRAMc_4N | TACACGACGCTCTTCCGATC TAGACTCCC |
| | _nP3s | TCAGCGGC (SEQ ID NO: 68) |
| NJ-506 | PE1s_GRAMc_6N | TACACGACGCTCTTCCGATC TAGACT |
| | _nP3s | CCCTCAGCGGC (SEQ ID NO: 69) |
| NJ-507 | PE1s_GRAMc_8N | TACACGACGCTCTTCCGATC TAGA |
| | _nP3s | CTCCCTCAGCGGC (SEQ ID NO: 70) |
| NJ-508 | PE1s_GRAMc_10 | TACACGACGCTCTTCCGATC TA |
| | N_nP3s | GACTCCCTCAGCGGC (SEQ ID NO: 71) |
| NJ-509 | PE1s_GRAMc_12 | TACACGACGCTCTTCCGATC |
| | N_nP3s | TAGACTCCCTCAGCGGC (SEQ ID NO: 72) |
| NJ-523 | GRAMc_Ion- | CCTCTCTATGGGCAGTCGGTGATtagactccctcagcggc |
| | P_nP3s | (SEQ ID NO: 73) |
| NJ-575 | GRAMc_test1_F | TTCACTAGTGATTCAGCAGGAGTGCCATCATGAT |
| | | TCATAAATAG (SEQ ID NO: 74) |
| NJ-576 | GRAMc_test1_R | ACACTACTCTCCAGCAGGTACTTAATATTTGAGG |
| | | TTACTCGTAG (SEQ ID NO: 75) |
| NJ-577 | GRAMc_test2_F | TTCACTAGTGATTCAGCAGCACCTGACCACTAGT |
| | | GGG (SEQ ID NO: 76) |
| NJ-578 | GRAMc_test2_R | ACACTACTCTCCAGCAGCACTTTGGAATCCAAAT |
| | | TTCCAG (SEQ ID NO: 77) |
| NJ-579 | GRAMc_test3_F | TTCACTAGTGATTCAGCAGCAAGTACAGCATTG |
| | | ACTGAGC (SEQ ID NO: 78) |
| NJ-580 | GRAMc_test3_R | ACACTACTCTCCAGCAGAGACAGAGCTGACACA |
| | | CAC (SEQ ID NO: 79) |
| NJ-589 | GRAMc_test8_F | TTCACTAGTGATTCAGCAGTTATTTTGCTTACAG |
| | | GGCCAG (SEQ ID NO: 80) |
| NJ-590 | GRAMc_test8_R | ACACTACTCTCCAGCAGGTGACACAGGAGCTTA |
| | | TATATATATAAGC (SEQ ID NO: 81) |
| NJ-591 | GRAMc_test9_F | TTCACTAGTGATTCAGCAGTACAATCCACCTACT |
| | | TAAAGTGTG (SEQ ID NO: 82) |
| NJ-592 | GRAMc_test9_R | ACACTACTCTCCAGCAGTTAAATAGAGACGGGG |
| | | TTTCAC (SEQ ID NO: 83) |
| NJ-691 | G5_1_F | TTCACTAGTGATTCAGCAGCCTTTCTAACTTGGG |
| | | TCATTTCTG (SEQ ID NO: 84) |
| NJ-692 | G5_1_R | ACACTACTCTCCAGCAGCTTTCTTTATCTACAGC |
| | | AAACAGG (SEQ ID NO: 85) |
| NJ-693 | G5_2_F | TTCACTAGTGATTCAGCAGCACAAGATACATGT |
| | | AGCTGAATTTAG (SEQ ID NO: 86) |
| NJ-694 | G5_2_R | ACACTACTCTCCAGCAGTATTTTTAGTAGAGACG |
| | | GGGTTTCAC (SEQ ID NO: 87) |
| NJ-695 | G5_3_F | TTCACTAGTGATTCAGCAGAAACCCTCTAGGTCC |
| | | TTTAAC (SEQ ID NO: 88) |
| NJ-696 | G5_3_R | ACACTACTCTCCAGCAGGGATTACAGGAATGTG |
| | | CCAC (SEQ ID NO: 89) |
| NJ-697 | G5_4_F | TTCACTAGTGATTCAGCAGAAAACACCACGTAG |
| | | TTTGGC (SEQ ID NO: 90) |
| NJ-699 | G5_5_F | TTCACTAGTGATTCAGCAGAAGCCAGCGTTGCC |
| | | CATC (SEQ ID NO: 91) |
| NJ-700 | G5_5_R | ACACTACTCTCCAGCAGGCCTCAGCCTCCTGAGT |
| | | AG (SEQ ID NO: 92) |
| NJ-701 | G5_6_F | TTCACTAGTGATTCAGCAGGTAAATCCAATCCCA |
| | | GGTTG (SEQ ID NO: 93) |
| NJ-702 | G5_6_R | ACACTACTCTCCAGCAGGCCACCATGTTTGGCTA |
| | | TTTTC (SEQ ID NO: 94) |
| NJ-705 | G3_1_F | TTCACTAGTGATTCAGCAGAGTTTTGGTATTTTA |
| | | ATACTCTTG (SEQ ID NO: 95) |
| NJ-706 | G3_1_R | ACACTACTCTCCAGCAGCATTGGTTAAGTGTAGC |
| | | AAAC (SEQ ID NO: 96) |
| NJ-707 | G3_2_F | TTCACTAGTGATTCAGCAGATCATTTTTCTTTCC |
| | | GAGATGTTG (SEQ ID NO: 97) |
| NJ-708 | G3_2_R | ACACTACTCTCCAGCAGTATTTTTTTTGAGATGG |
| | | AGTTTCGC (SEQ ID NO: 98) |
| NJ-709 | G3_3_F | TTCACTAGTGATTCAGCAGCCCGTTCCACAAGG |
| | | ATCTGTG (SEQ ID NO: 99) |
| NJ-710 | G3_3_R | ACACTACTCTCCAGCAGCTCCGGAATAGCTGGG |
| | | ATTAC (SEQ ID NO: 100) |
| NJ-711 | G3_4_F | TTCACTAGTGATTCAGCAGTCTCCTTATAAATAT |
| | | CTTTCACTTCC (SEQ ID NO: 101) |
| NJ-712 | G3_4_R | ACACTACTCTCCAGCAGAGAATTAAGGGGGAAA |
| | | AGTTG (SEQ ID NO: 102) |
| NJ-713 | G3_5_F | TTCACTAGTGATTCAGCAGGTGGAATCTGGAGG |
| | | CCAG (SEQ ID NO: 103) |
| NJ-714 | G3_5_R | ACACTACTCTCCAGCAGTTGTTGGCTCTGGTTTT |
| | | TCTTTG (SEQ ID NO: 104) |
| NJ-717 | L1_1_F | TTCACTAGTGATTCAGCAGCTTCCTTCCTACCTT |
| | | CTTTTTC (SEQ ID NO: 105) |
| NJ-718 | L1_1_R | ACACTACTCTCCAGCAGAAAACCTGGGAGTCCC |
| | | AAAG (SEQ ID NO: 106) |
| NJ-719 | L1_2_F | TTCACTAGTGATTCAGCAGACCTTCTTACTTCTT |
| | | AAGGGGG (SEQ ID NO: 107) |
| NJ-720 | L1_2_R | ACACTACTCTCCAGCAGTCTGCGAGTCCTCCTCT |
| | | TCTTTG (SEQ ID NO: 108) |
| NJ-723 | L1_4_F | TTCACTAGTGATTCAGCAGGCAACCAGCTTGGA |
| | | AATTTCTC (SEQ ID NO: 109) |
| NJ-724 | L1_4_R | ACACTACTCTCCAGCAGAGACTTCGACTTCTTCG |
| | | GATG (SEQ ID NO: 110) |
| NJ-727 | L1_6_F | TTCACTAGTGATTCAGCAGAACTAACATGGCTG |
| | | ATGCCTTG (SEQ ID NO: 111) |
| NJ-728 | L1_6_R | ACACTACTCTCCAGCAGTATTTGGTTTGCTTAGA |
| | | GTCCTCCTCTG (SEQ ID NO: 112) |
| NJ-729 | EGFP_5p_F | ATGGTGAGCAAGGGCGAG (SEQ ID NO: 113) |
| NJ-730 | EGFP_3p_R | TTATCTAGATCCGGTGGATC (SEQ ID NO: 114) |
| NJ-731 | EGFP_GRAMc_ | GATCCACCGGATCTAGATAAGCCTCTAGACTCC |
| | gibson_F | CTCAGCGGCGC (SEQ ID NO: 115) |
| NJ-732 | EGFP_GRAMc_ | CTCGCCCTTGCTCACCATTTGTGATTCACTTGTA |
| | gibson_R | AGATGACG (SEQ ID NO: 116) |
| Adapter | GRAMcP1s-SE.fa | TCACTAGTGATTCAGCA (SEQ ID NO: 117) |
| Trimming | | |
| Adapter | GRAMcP2s-SE.fa | ACACTACTCTCCAGCA (SEQ ID NO: 118) |
| Trimming | | |
| Adapter | GRAMcP3s-SE.fa | ACTCCCTCAGCGGCGCGC (SEQ ID NO: 119) |
| Trimming | | |
| Adapter | GRAMcP4s-SE.fa | AGTCCGACGATCCAGCA (SEQ ID NO: 120) |
| Trimming | | |
| Adapter | GRAMcP1sr-SE.fa | CTGCTGAATCACTAGTGA (SEQ ID NO: 121) |
| Trimming | | |
| Adapter | GRAMcP2sr-SE.fa | CTGCTGGAGAGTAGTGT (SEQ ID NO: 122) |
| Trimming | | |
| Adapter | GRAMcP3sr-SE.fa | GCGCGCCGCTGAGGGAGT (SEQ ID NO: 123) |
| Trimming | | |
| Adapter | GRAMcP4sr-SE.fa | TGCTGGATCGTCGGAC (SEQ ID NO: 124 |
| Trimming | | |

### GRAMc Library Characterization by ILLUMINA® Paired-End Sequencing

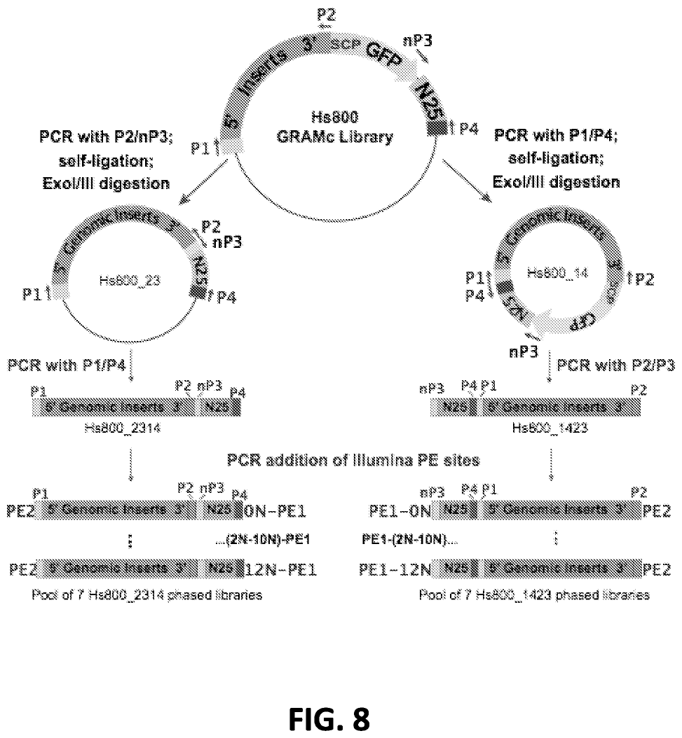

Sequencing library: To identify inserts and associated barcodes in individual reporter constructs, paired-end sequencing was used with the NextSeq500 platform. Sequencing the Hs800_GRAMc library on the ILLUMINA® platform was a problem for two reasons: i) the length of reporter constructs was too long for paired-end sequencing and ii) lack of diversity in the adapter sequences is incompatible with ILLUMINA® platform. To solve the length problem, the length of the constructs was reduced by bringing inserts and N25 barcodes closer by deleting either SCP-GFP region or the vector backbone by inverse PCR and self-ligation. To solve the low sequence diversity problem, a set of phased primers (Wu, et al. BMC microbiology 15.1 (2015): 125) was used to artificially increase sequence diversity. Generation of two different populations of sequencing libraries that lack either SCP-GFP region or the vector backbone also increases sequence diversity at the adapter region (FIG. 8).

In this example, constructing a sequencing library begins with cutting 500 ng of the maxi-prepped plasmids with Cas9 (NEB® M0386) using sgRNAs against either the vector backbone or the GFP ORF. Both sgRNAs were predicted to have 7 off-target sites in the human genome (crispr.mit.edu). Primer pairs, NJ-179/NJ-183 and NJ-180/NJ-183, were used to produce templates for in vitro transcription of sgRNAs that respectively target the backbone and GFP. The primer sequences are available in Table 3. The CRISPR-cut plasmid libraries were mixed with an equimolar amount of uncut plasmid libraries. Inverse PCR of 5 ng of the GFP-cut linear library mixture was performed using NJ-209 and NJ-141 (denoted as “Hs800_23”) to remove the SCP-GFP region, and inverse PCR of 5 ng of the backbone-cut linear library mixture was performed using NJ-208 and NJ-142 (denoted as “Hs800_14”) to remove the vector backbone. Q5® High-Fidelity DNA Polymerase (NEB®) for PCR was used. A total of 20 replicates were prepared per template/primer pairs. Respective replicates were combined, column concentrated, gel isolated, and AXYGEN® bead cleaned. Respective amplifications were self-ligated at a concentration of 75 ng in 350 μL of 1× T4 DNA Ligase buffer with 3 of concentrated T4 ligase overnight at 20° C., supplemented with 20 U each of exonuclease I and exonuclease III at 37° C. for 1 hr, followed by incubation with Proteinase K for 10 minutes at 37° C. Ligates were AXYGEN®-bead cleaned and eluted in 30 μL of water.

To amplify insert::N25 cassettes, from the circularized first round PCR products, 4 replicates containing 2 ng of Hs800_14 ligates were amplified using NJ-209 and NJ141 (hereinafter denoted as Hs800_1423), and 4 replicates containing 2 ng Hs800_23 ligates were amplified using NJ-208 and NJ142 (hereinafter denoted as Hs800_2314) with an annealing temperature of 60° C. and an extension time of 90 seconds for a total of 8 cycles. Products were column cleaned, gel isolated, and bead cleaned for subsequent PCR amplification to add PE adapter sequences for ILLUMINA® sequencing.

To increase diversity of the Hs800_1423 and Hs800_2314 sequencing libraries for sequencing on the ILLUMINA® platform, each library (Hs800_1423 and Hs800_2314) was amplified using 7 different phased PE1-containing primers. For the Hs800_1423 library, 2 ng of template was used per each separate reaction with the PE2-containing primer NJ-401 and each of the following partial PE1-containing primers: NJ-400, NJ-504, NJ-505, NJ-506, NJ-507, NJ-508, and NJ-509 with an annealing temperature of 60° C. and an extension time of 90 seconds for a total of 7 cycles. For the Hs800_2314 library, 2 ng of template were used per each separate reaction with the PE2-containing primer NJ-403 and each of the following partial PE1-containing primers: NJ-402, NJ-498, NJ-499, NJ-500, NJ-501, NJ-502, and NJ-503 with an annealing temperature of 60° C. and an extension time of 90 seconds for a total of 7 cycles. The phased PE1 primers can be pooled before PCR amplification to simply the procedure. Individual amplifications were column cleaned, gel isolated, and AXYGEN®-bead cleaned. Each of the 7 phased Hs800_1423 libraries were amplified using NJ-497 and NJ-401 to complete the PE1 adapter sequence. Each of the 7 phased Hs800_2314 libraries were amplified using NJ-497 and NJ-403 to complete the PE1 adapter sequence. For each amplification, 2 ng of respective library templates were amplified in 6 cycles of PCR with an annealing temperature of 60° C. and an extension time of 90 seconds. Libraries were again purified, gel isolated, and AXYGEN®-bead cleaned. Equimolar amounts of the 14 phased libraries (7 from each direction) were combined to the 90% of the sequencing pool plus 10% PhiX control and used for paired-end sequencing. The sequences of primers are available in Table 3.

Trimming adapter sequences from inserts and barcodes: The 5′- and 3′-ends of an insert and its associated N25 barcode were extracted from each pair of sequence reads. Trimmomatic (Bolger, et al. Bioinformatics 30.15 (2014): 2114-2120) was used to remove adapter sequences and seqtk (github.com) to reverse complement sequences. To extract the 5′-end and 3′-end of an insert, P1 and P2 adapters, respectively, were trimmed. To extract N25 barcodes, depending on the orientation of a sequence read, a P3 or P4 adapter was trimmed first, reverse complemented the trimmed sequence, and trimmed P4 or P3 adapter. Paired-end reads that failed to trim any adapter sequence were abandoned. Note that in the case of N25 barcode sequences, 1 bp was retained from each adapter, resulting in 27 bp reads. Adapter sequences used for trimming are available in Table 3.

Mapping sequence reads and identification of inserts in the human genome: To identify inserts, extracted 5′- and 3′-ends of inserts were mapped on to the GRCh38/hg38 assembly (downloaded from genome.ucsc.edu). The Burrows-Wheeler Alignment tool (BWA) (Li, et al. Bioinformatics 25.14 (2009): 1754-1760) was used to map sequences with the following command: “bwa mem -W 1500.” Mapped pairs of reads that spanned >1,500 bp or <300 bp were abandoned. When two mapped inserts overlapped, their mid-points were within a 20 bp range, and both ends were within a 50 bp range, they were combined into one insert, taking the coordinates that maximize its length.

Clustering N25 barcodes: To identify reads from the same barcode, the extracted barcode reads were clustered based on the following procedure: i) representative reads were generated by filtering redundant reads by using the Khmer software package (Crusoe, et al. F1000Research 4 (2015)) with the command: “normalize-by-median.py -C 1 -k 25 -N 5 -x 2.5e9;” and ii) the entire set of barcode reads was matched against the representative reads using the BWA software (Li, et al. Bioinformatics 25.14 (2009): 1754-1760) with the command: “bwa aln -n 2 -o 2 -e -1 -M 3 -O 11 -E 8 -k 1-l 6.” Barcode reads that did not match any of the representative reads were added to the representative reads file, and the BWA search was repeated. Reads for the same barcodes were identified by single-linkage-clustering, and each cluster was assigned a unique barcode cluster (bcl) number. A new file of representative reads with the bcl numbers was generated for future use (see below, GRAMc assay in HepG2: Matching barcode reads to barcode clusters).

Associating genomic inserts with barcode clusters (bcls): Although each barcode read is inherently connected to reads from an insert in paired-end reads, a minor fraction of bcls were associated with more than one of the identified genomic inserts. The main reason for this ambiguity is highly similar duplicated regions in the genome. The assignment of a bcl was forced for an insert that had the most reads for the bcl. If ≥2 inserts had the same number of reads for a bcl, the bcl was not assigned to any insert.

### GRAMc Assay in HepG2

Cell culture: HepG2 cells (ATCC HB-8065) were grown under supplier-recommended conditions of EMEM supplemented with 10% fetal bovine serum without antibiotics. HepG2 cells were used within no more than 16 passages from receipt for all experiments. All experiments were performed in cells that underwent a minimum of 5 passages from thawing because reporter expression in cells of <5 passages versus cells of ≥5 passages were different.

Genome-scale transfection and lysate collection: For each genome-scale transfection batch, 10⁷ cells were seeded in 30 ml media in each of 10×150 mm culture dish (100 M cells) and allowed to attach for 30 hours. Cells were transfected with 100 μg of the Hs800_GRAMc library using 100 μL of DNA-IN® for HepG2 reagent (MTI-Globalstem) in 4 ml of OPTI-MEM® (THERMOFISHER®) prepared in 2×2-mL siliconized tubes according to the manufacturer's protocol. A total of 10 10×150 mm dishes were used to collect about 200 M cells per batch.

For collection, cells were washed with 1×PBS for 26 hours after transfection and were collected by scraping in 2.4 mL RNA-STAT-60 (AMSBIO®) per plate. Lysates were combined and prepared according to the manufacturer's protocol with the addition of a second 70% ethanol wash.

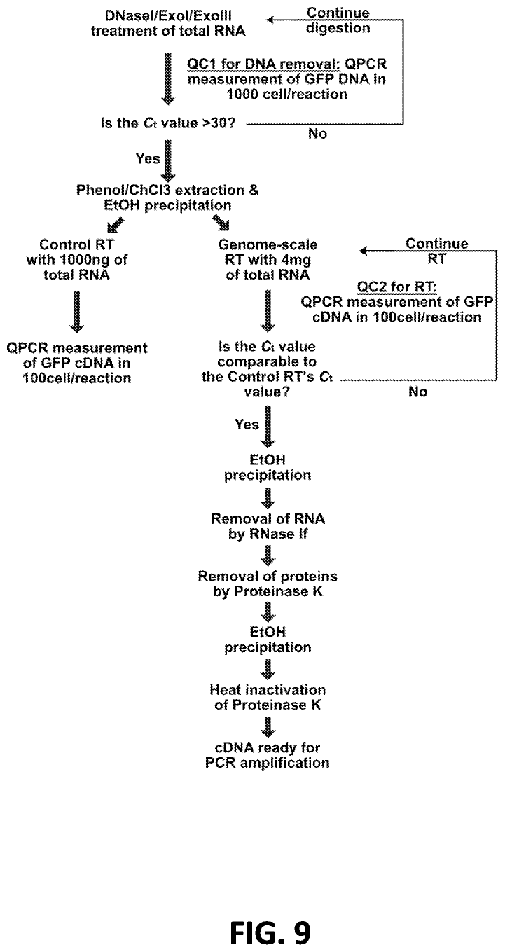

RNA preparation and cDNA synthesis: The protocol focuses on two parameters: i) comprehensively removing contaminated DNAs in RNA sample and ii) maximizing the efficiency of reverse transcription (RT) with a large quantity (about 4 mg) of total RNA. Complementing DNase I with a cocktail of exonuclease I and III comprehensively removes both double-strand and single-strand contaminating DNA, as DNase I is less efficient against single stranded DNA. To cost-efficiently maximize RT, 15 times more RNA was used than manufacturer's recommended maximal input RNA without compromising cDNA yield in RT reaction. A schematic of the procedure is available in FIG. 9.

To remove contaminated DNA, isolated total RNA (about 4 mg) was resuspended in 1.7 mL of nuclease-free water and digested for a minimum of 4 hours at 37° C. in a 2 mL reaction containing 1× DNase I Buffer, 100 U of DNase I (NEB® M0303), and 900 U each of exonuclease I (ExoI) and exonuclease III (ExoIII). The progress of DNA removal was monitored by QPCR against the GFP ORF (NJ-443 and NJ-444). For this quality control step, a diluted sample of RNA was heat inactivated at 80° C. for 20 minutes and loaded at an equivalent volume of about 1000 cell/well. As needed, DNase digestion was allowed to proceed overnight until the QPCR Ct value become greater than 30. Following digestion, nucleases were removed by extraction with Phenol:Chloroform:Isoamyl alcohol (25:24:1) and ethanol precipitated overnight at −20° C. followed by two washes with 75% ethanol. RNA was resuspended in 1 mL of RNase-free water.

As a quality control for reverse transcription (RT), an equivalent volume of the total RNA containing about 4000 cells (about 1 μg) was used for cDNA synthesis using the High Capacity cDNA Reverse Transcription Kit (APPLIED BIOSYSTEMS® 4368813) following the manufacturers protocol with the addition of 5 pmol of a GRAMc library specific RT oligo (NJ-489) and used as the standard for maximum cDNA synthesis from transcripts.

The remaining total RNA (about 4 mg) was diluted to 1.420 mL, and 2000 pmol of GRAMc_RT_oligo (NJ-489) was added. The RNA/primer mixture was incubated at 65° C. for 1 minute and chilled on ice, followed by addition of 200 μL of 10× High Capacity buffer, 80 of 10 mM dNTP and 100 μL of Multiscribe without using random oligomers. The reaction was incubated for 10 minutes at room temperature and then for 4 hours at 37° C. The progression of the genome-scale cDNA synthesis was monitored via QPCR against GFP in comparison to the standard RT control using an equivalent volume of 100 cells/well. Reactions were allowed to proceed until the Ct value became similar to the standard RT reaction. If needed, the reactions were spiked with M-MuLV Reverse Transcriptase (NEB® M0253) and additional dNTPs and allowed to proceed overnight.

Upon completion of the RT reaction, the samples were ethanol precipitated to reduce the volume. RNA/cDNA was resuspended and digested with 1000 U of RNase If (NEB® M0243) in a 500 μL reaction with 1× NEBUFFER® 3 at 37° C. overnight. For removal of excess protein, 1 μL of Proteinase K solution was added to the reaction and incubated at 37° C. for 15 minutes. cDNA was ethanol precipitated overnight at −20° C. with glycogen as a carrier and washed 3× with 80% ethanol. cDNA pellets were resuspended in 200 μL of water and heated to 95° C. for 10 minutes to destroy residual Proteinase K. A sample of the cDNA library was subjected to quality control by QPCR.

Preparation of expressed N25 barcodes for NGS: The entire pool of expressed N25s was amplified using primers NJ-141 and NJ-142 in 8 replicates of a 50 μl Q5® PCR reaction using an annealing temperature of 62° C. and an extension time of 1 minute for a total of 8 cycles. Replicates were combined for each batch. A 50 μL aliquot was processed from each batch as follows: unwanted long DNAs were bound using a 0.5× volume of AXYGEN® beads for 20 minutes at room temperature. The desired short amplicons (65 bp) from the supernatant were further purified for each batch using duplicate Zymo column and each eluted in 20 μL of water. To prepare amplicons for sequencing expressed barcodes, 2 ng of 1st round-amplified and cleaned N25 barcodes were subjected to another 9 cycles of amplification with NJ-141 and NJ-142. To prepare amplicons for sequencing the input library, 2 ng of the input library was amplified in 9 cycles of PCR from a mixture of uncut/CRISPR backbone-cut/CRISPR GFP-cut plasmid library template using the NJ-141 and NJ-142 primers.

Sequencing libraries were prepared both for IONTORRENT® Proton sequencing (Batch 1: NJ197 and NJ-523; Batch 2: NJ-198 and NJ-523) and ILLUMINA® NextSeq500 sequencing (14 phased libraries using NJ-400/NJ-504/NJ-505/NJ-506/NJ-507/NJ-508/NJ-509 with NJ364 or NJ-402/NJ-498/NJ-499/NJ-500/NJ-501/NJ-502/NJ-503 with NJ-399). For all of these amplifications, an annealing temperature of 65° C. and an extension time of 20 seconds was used for a total of 6 cycles. The sequences of primers are available in Table 3.

Matching barcode reads to barcode clusters (bcls): The goal of this step is to count the number of barcode reads from either expressed barcodes or the input library for each barcode cluster (bcl). Adapter-trimmed barcode reads were matched to the representative barcode reads established in the above by using BWA search with the same command as above. When a barcode read matched more than one bcl, each match was counted to the respective bcls. Because the same procedure was applied to both expressed barcodes and the input library, the effect of multiple counting of a barcode read is neutralized.

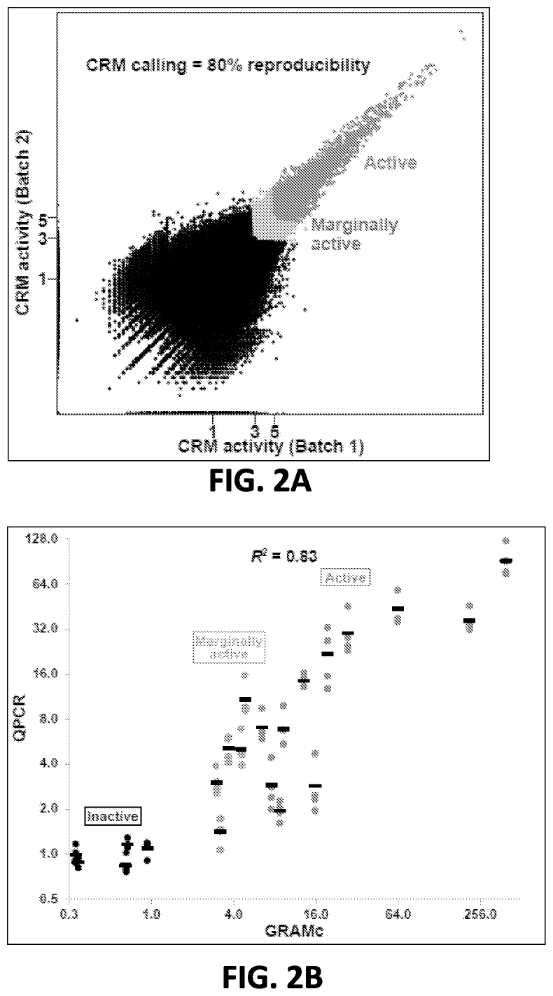

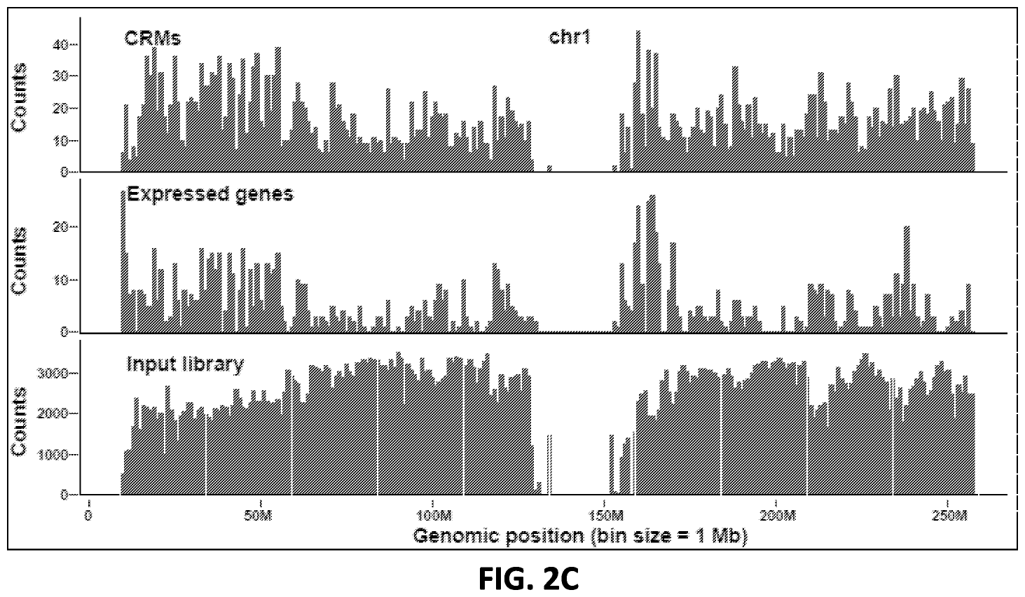

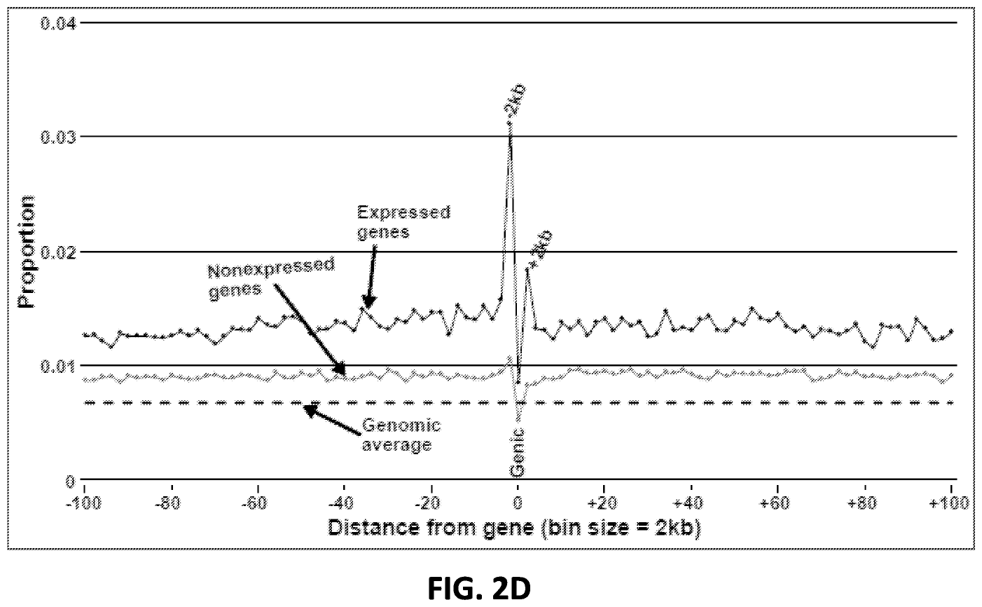

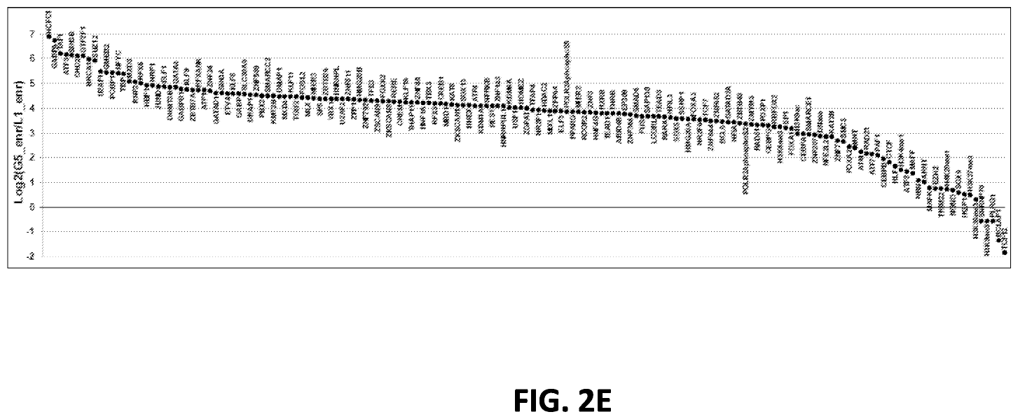

Computation of CRM activity: This step computes cis-regulatory activity of each insert based on the number of reads for each bcl that are counted from expressed barcodes and the input library. When an insert is associated with ≥2 bcls (99% of inserts), the read counts for all bcls for the insert were combined. First, to avoid false positive CRMs due to too low input counts, inserts with ≥10 counts from the input library or ≥50 counts of expressed barcodes for both batches of experiments were retained. This filtering resulted in 9,339,996 inserts that met the retention criteria. Second, read counts for expressed barcodes were divided by the read counts for the input library, and the resulting numbers were rank ordered. The middle 30% of data were used to compute the background activity (bg) (e.g., 26). CRM activities were further normalized to the background activity. An insert was considered a CRM when at least one batch showed ≥5×bg and another showed ≥4.5×bg (90% of 5×bg). A total of 54,115 inserts were identified that passed the criteria. After removing inserts with ≥95% identical sequences in other part of the genome and merging overlapping CRMs, the final set contained 41,216 unique and non-overlapping CRMs. A scatter plot is shown in FIG. 2A and was generated by using ggplot2 (Wickham. ggplot2: Elegant Graphics for Data Analysis, Springer-Verlag New York, 2009) in the R package (cran.r-project.org) using 500,000 randomly selected inserts.

### Genomic Distribution of CRMs

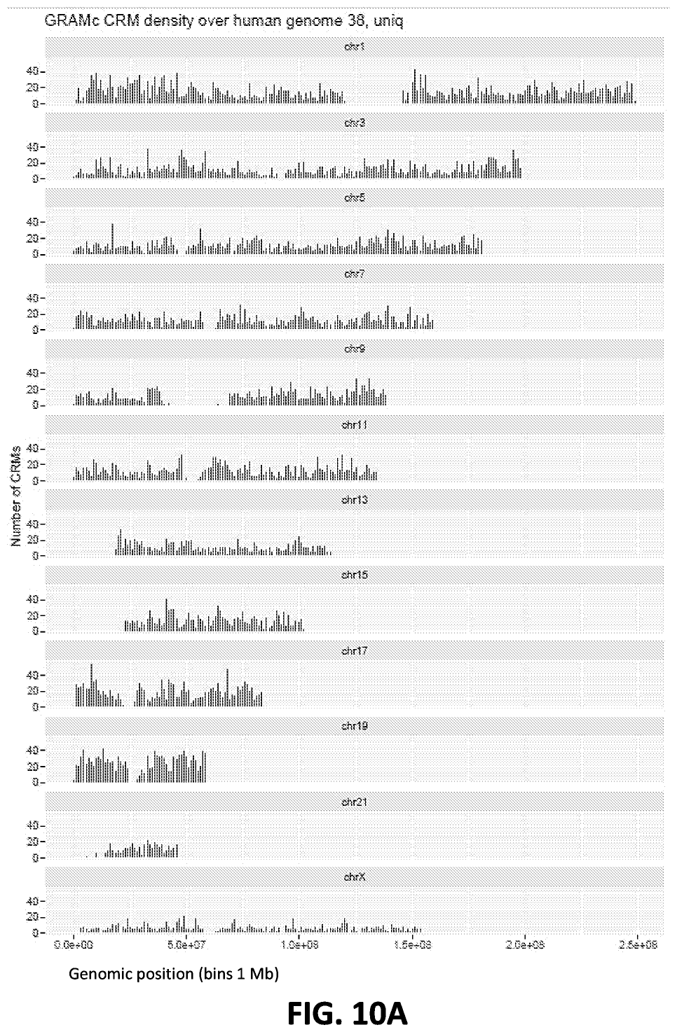

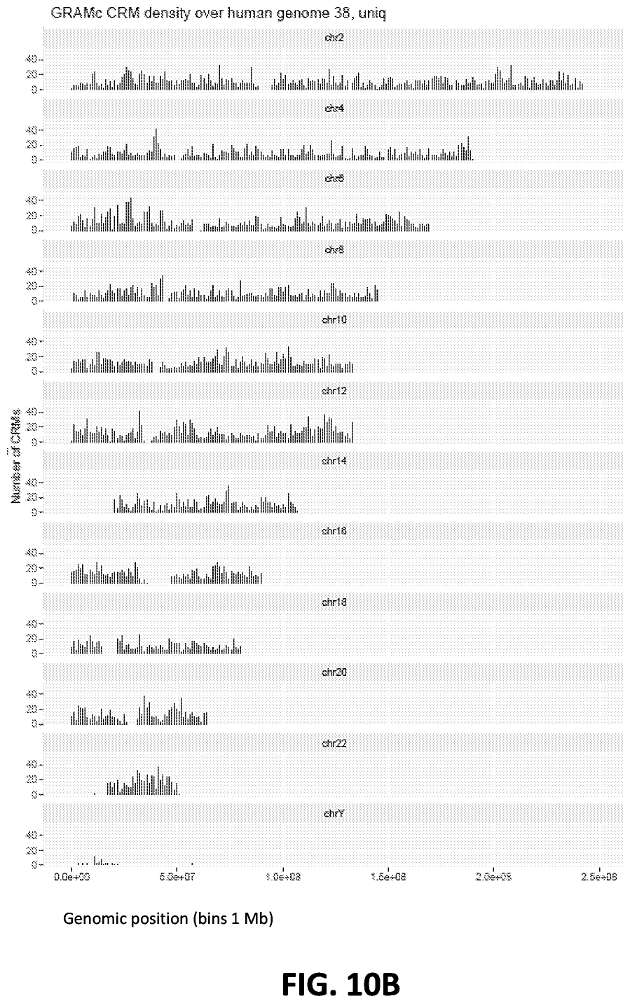

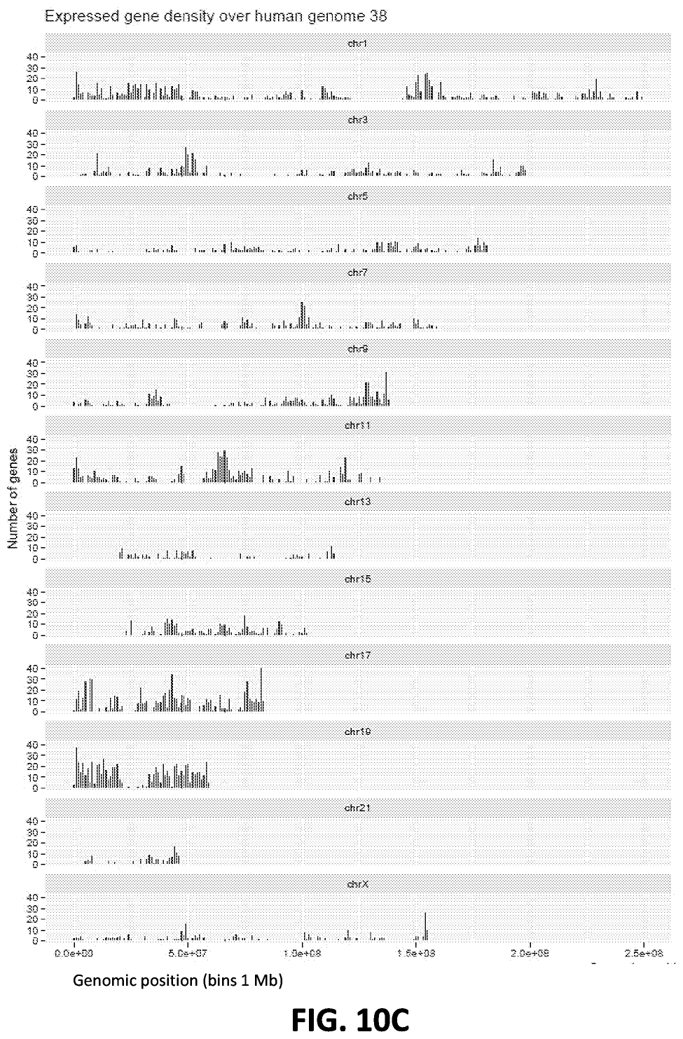

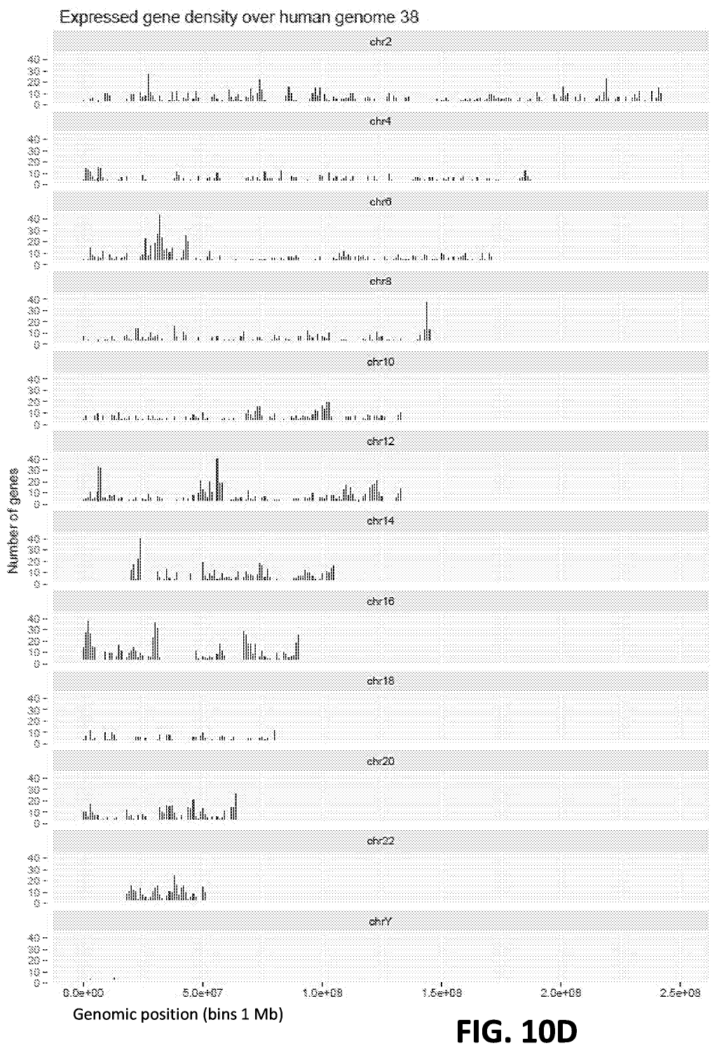

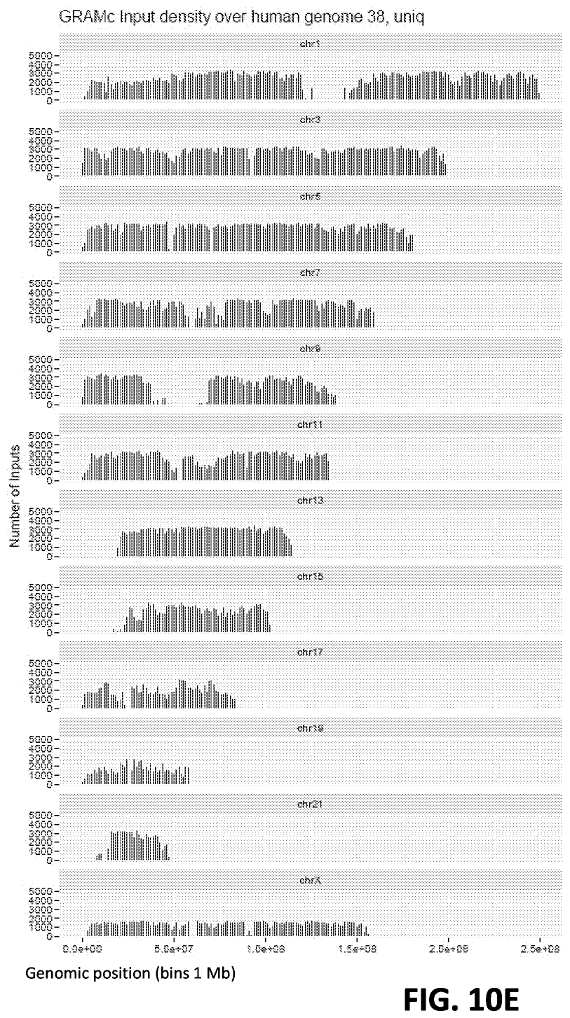

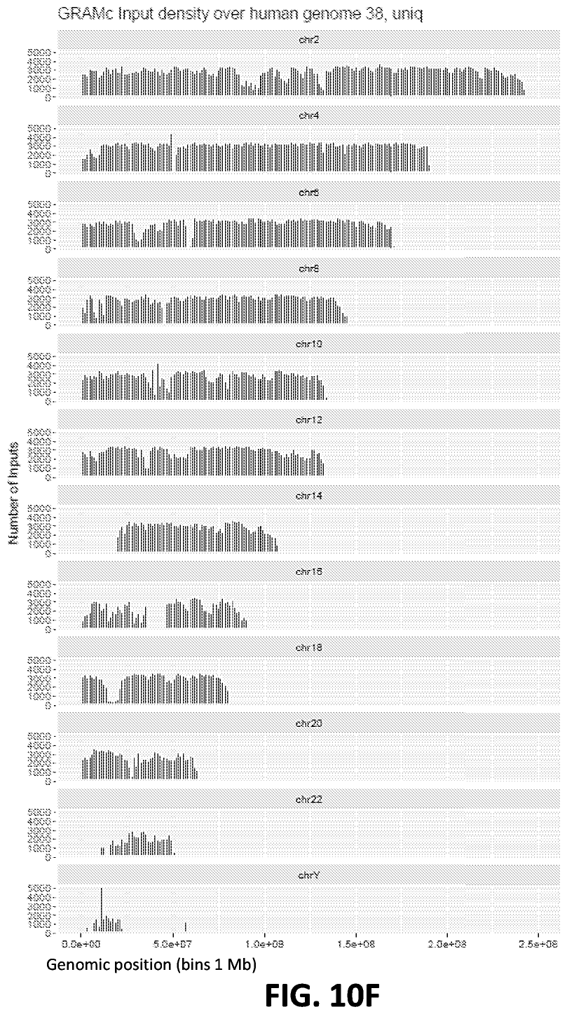

To compare genomic locations of CRMs and genes, publicly available gene annotation file “GRCh38.89.gff3” from ftp.ensembl.org and RNA-seq data for HepG2 cells “ENCFF861GCR and ENCFF640ZBJ” from encodeproject.org were used. Genes with FPKM≥1 in both RNA-seq data were considered “expressed”. To generate the maps shown in FIGS. 2C and 10A-10F, Grid Graphics Package (Murrell, graphics. CRC Press, 2016) in R was used with a bin size of 1 Mb.

To compute enrichment of CRMs in genomic regions with respect to genes (FIG. 2D), insert/CRMs that span more than a 2 kb window were assigned to a window that overlaps most with the insert. Genomic coordinates of the 5′-end and 3′-end of a gene were extracted from a GRCh38.89.gff3 file. An insert/CRM was counted only once for a gene but was allowed to be counted multiple times for different genes.

### One-by-One Reporter Assay for Validation

Making individual reporter constructs: Twenty genomic regions (11 CRMs, 5 marginally active regions, and 4 inactive regions) were individually amplified by PCR and cloned into a pre-barcoded SCP-GRAMc vector (Guay, et al. Developmental biology 422.2 (2017): 92-104) by GIBSON ASSEMBLY® (Gibson, et al. Methods in enzymology 498 (2011): 349-361). Primers were used to amplify inserts contain flanking sequences that overlap with adapter sequences present in the vector. Each assembly was performed using a 2 μL NEBUILDER® HiFi Assembly reaction. Assembly reactions were used to transform Mix and Go DH10B competent cells (Zymo Research T3019), and positive clones were identified by colony PCR. Endotoxin-free plasmids were prepared (Zymo Research D4208T).

The pre-barcoded SCP-GRAMc vector was further used to generate an EGFP internal control vector for use in QPCR of GFP reporter expressions for individual clones. For this step, the vector was amplified by inverse PCR with NJ731 and NJ732. The EGFP ORF from pEGFP-C1 was amplified using NJ729 and NJ730 and assembled to the SCP-GRAMc vector using GIBSON ASSEMBLY® at a ratio of 2:1 using the NEBUILDER® HiFi Assembly master mix. The GFP ORF used in the GRAMc vector is different from the commonly used EGFP ORF, and the two GFPs can be differentially detected by QPCR. The sequences of primers are available in Table 3.

Individual reporter assay to validate GRAMc results: HepG2 cells were seeded at about 60K cells per well in a 24-well plate in 500 μL of EMEM supplemented with 10% FBS. For consistency with the genome-scale assay, cells were used between passages 12 and 15 from receipt from ATCC and at least 7 passages after recovery. The cells were allowed to attach for 24 hours and transfected with a mixture of 50 μL OPTI-MEM®, 200 ng of GFP-containing individual test plasmids, 200 ng of a SCP-EGFP control vector, and 1.2 μL DNA-IN® reagent. After 26 hours (about 80-85% confluency, consistent with the genome-scale assay), cells were washed twice in DPBS, collected in 300 μL of DNA/RNA lysis buffer (ZymoResearch), and gDNA and total RNA for each sample was purified using Zymo II columns with binding and washing as per the manufacturer's protocol. RNA was eluted in 34 μL of water. Half of the total RNA for each sample was treated in a 20 μL Turbo DNase reaction (THERMOFISHER®) for 1 hour at 37° C. The reactions were terminated with 2 μL of DNase inactivation reagent (THERMOFISHER®). Half of the DNase-treated RNA was used in a 20 μL 1× High-Capacity cDNA synthesis reaction with an additional 10 pmole of GRAMc_RT_oligo (NJ-489) and RNase inhibitor. QPCR was performed against GFP and EGFP on a total gDNA equivalent of 1/40,000 of the original sample, a non-RT control equivalent of 1/40 of the total RNA sample, and a cDNA equivalent of 1/160 of the original sample. GFP expression driven by individual test fragments were normalized to the internal control (EGFP expression, NJ404/NJ405). The sequences of QPCR primers are available in Table 3.

### Relative Enrichment of ENCODE Annotations in CRMs Versus Inactive Inserts

ENCODE ChIP-seq files were obtained from encodeproject.org. Overlap between CRMs and individual ENCODE data was computed using bedtools (Quinlan, et al. Bioinformatics 26.6 (2010): 841-842) with the command: “bedtools jaccard -f 1E-09 -F 1E-09.” The relative enrichment of ENCODE annotations in CRMs was computed in the following procedure. i) First, the genomic proportion of overlapping base pairs between CRMs and an ENCODE annotation was computed. ii) Randomly expected overlap by multiplying genomic proportions of the two datasets was computed. iii) The result from i) was divided by result from ii) to compute the enrichment. iv) Following the same procedure, enrichment of the same ENCODE annotation in inactive regions (L1 group) was computed. v) Relative enrichment was computed by taking the ratio of iii and iv.

### Motif Enrichments in CRMs and Predicted Strong Enhancers

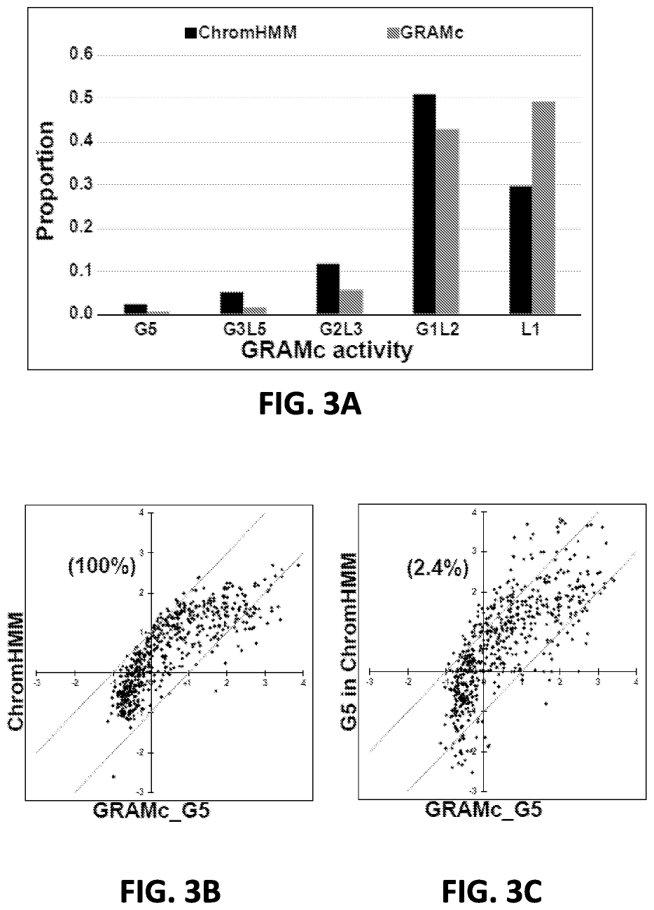

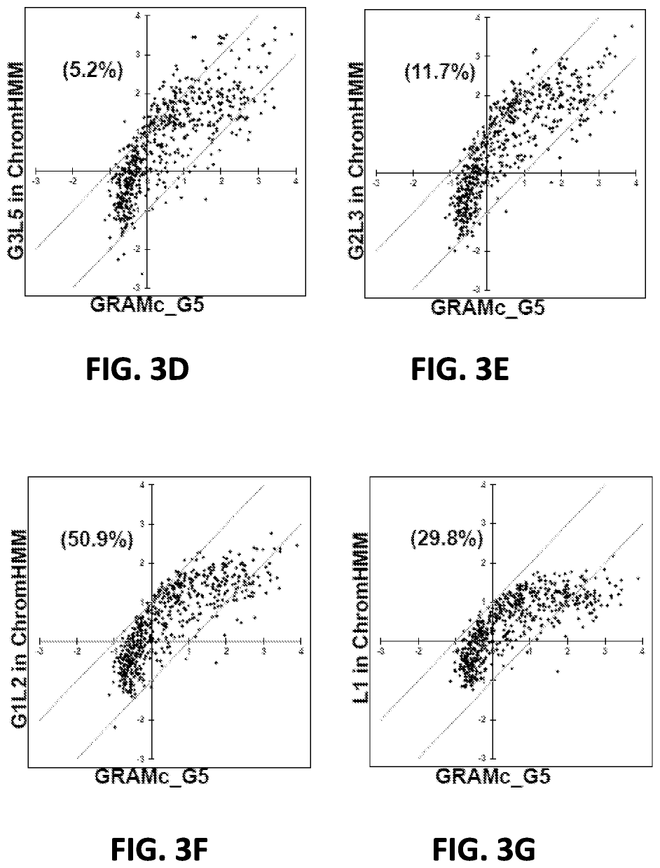

Selection of GRAMc inserts: Strong enhancers for HepG2 as predicted by ChromHMM (Ernst, et al. Nature 473.7345 (2011): 43; Ernst, et al. Nature biotechnology 28.8 (2010): 817) were compared to GRAMc data for CRM activity and motif enrichment. Genomic coordinates of chromatin states were converted via liftOver (Hinrichs, et al. Nucleic acids research 34.suppl_1 (2006): D590-D598) to hg38. First, nonoverlapping GRAMc inserts that ≥90% overlap in length with predicted strong enhancers were randomly selected. This selection process yielded 18,898 GRAMc inserts that correspond to predicted strong enhancers. This data was utilized to generate FIG. 3A.

To compare motif enrichment, another 18,898 nonoverlapping GRAMc CRMs (≥5×bg or G5) were randomly sampled without considering predicted enhancers. As a negative control, 37,796 nonoverlapping inactive (≤1×bg or L1) inserts were also sampled.

Motif enrichment survey: To survey putative transcription factor binding site (TFBS) motifs, the 75,592 inserts sampled were analyzed simultaneously. The HOCOMOCOv10 database (Kulakovskiy, et al. Nucleic acids research 44.D1 (2015): D116-D125) and FIMO software (Cuellar-Partida, et al. Bioinformatics 28.1 (2011): 56-62; Bailey, et al. Nucleic acids research 37 (2009): W202-W208) were used with an E-value cutoff of 1E-5. The abundance of each motif is the proportion of motif-harboring inserts for a given set. Relative motif enrichment was computed by dividing the abundance of a motif in CRMs or predicted enhancers by the abundance of the same motif in the negative control set.

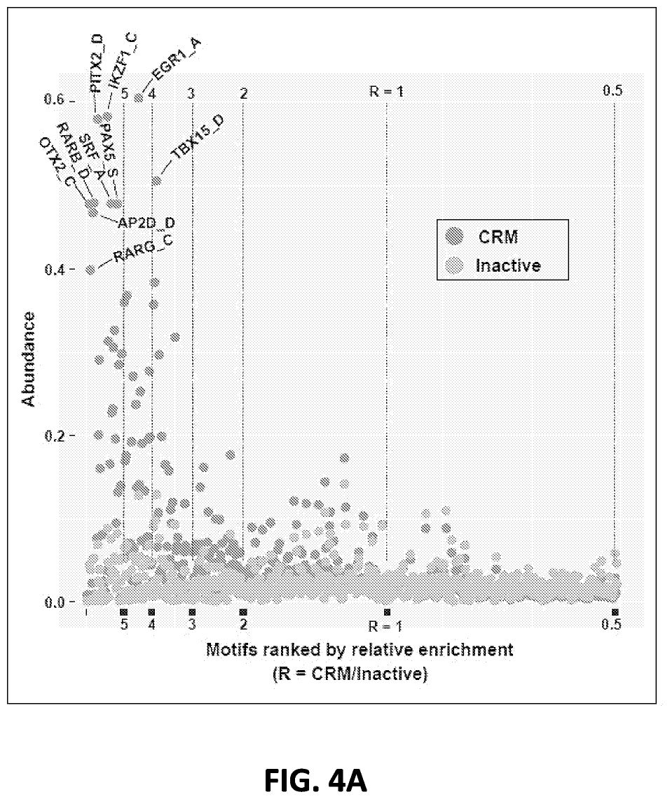

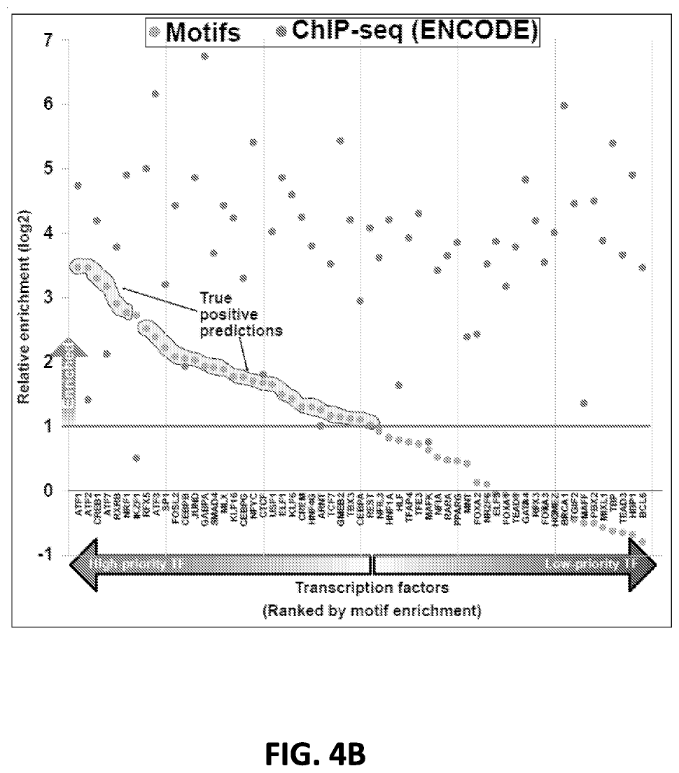

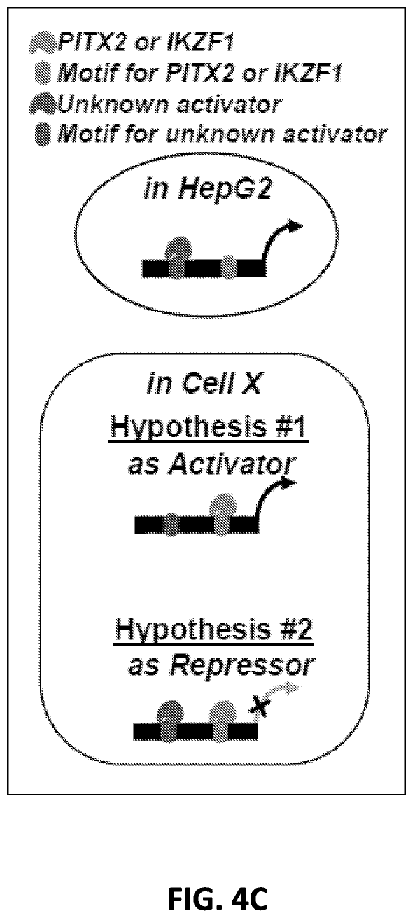

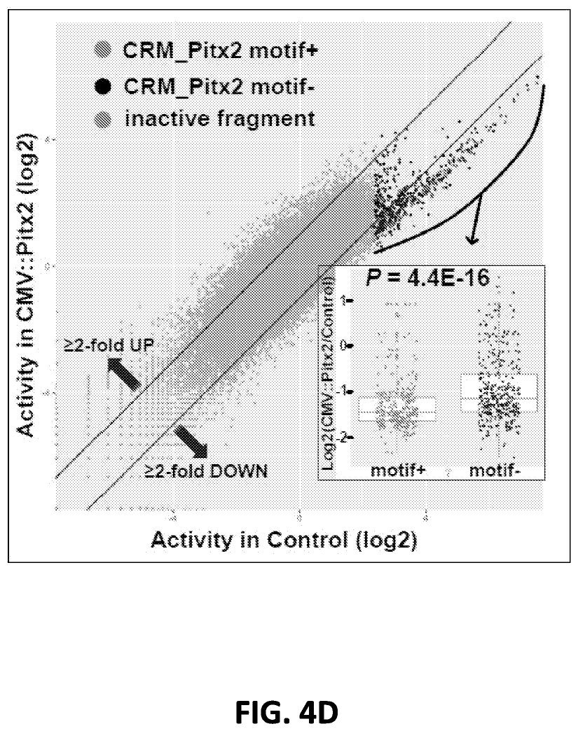

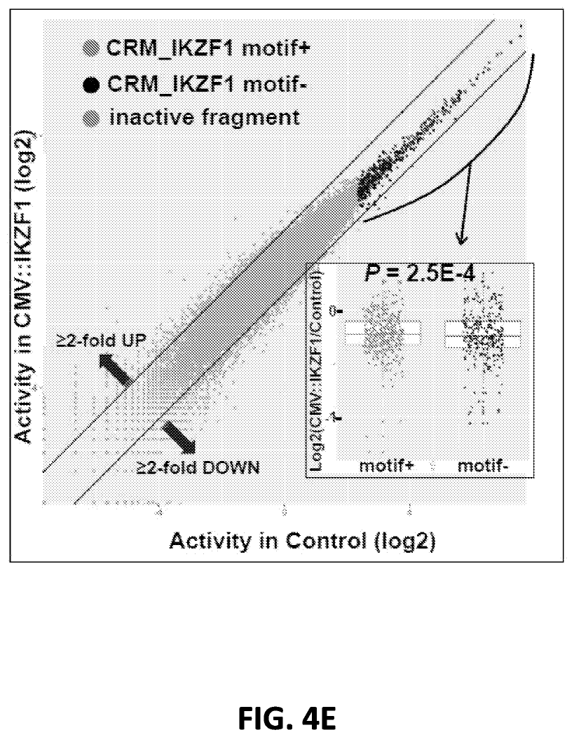

Comparison of enrichments of motifs and ChIP-seq peaks in CRMs: Fifty-eight common transcription factors between the HOCOMOCOv10 and ENCODE ChIP-seq data were identified by name. The relative enrichment scores computed were used to generate FIG. 4B.

### Measuring the Effect of Gene Ectopic Expression on CRMs

Preparation of random sub-sets of the GRAMc library: To obtain small-scale subsets of the GRAMc library for perturbation experiments by ectopic expression of pitx2 or ikzf1, about 50 μL of frozen glycerol stock was diluted into 2 ml of LB media, recovered with orbital shaking 250 RPM at 37° C. for 20 minutes. A series of 2-fold dilutions were prepared, 1/100th of which was used for 2 10-fold dilutions for plating and colony counting, and the remainder of each 2-fold diluted culture was used to seed 150 ml LB-Amp cultures for overnight growth. Cultures that were estimated to contain about 80,000 colonies (80 K library) were processed using the ZYMOPURE® Plasmid Maxiprep Kit.

Perturbation assay for the 80 K construct library: Cells were seeded in duplicates of about 2 M cells per 10 cm² plate for transfection with each of 3 co-transfections: 80 K library+CMV::pitx2 (Genscript OHu17480D), 80 K library+CMV::IKZF1 (Genscript OHu28016D), and 80 K library+CMV::EGFP (Clontech pEGFP-C1). Cells were cultured for about 24 h prior to transfection. Cells were co-transfected with 9 μg of the 80 K library and 3 μg of the respective expression vector using 36 μL of DNA-IN® for HepG2 reagent (MTI-Globalstem) and 1.2 ml of OPTI-MEM® (THERMOFISHER®) prepared according to the manufacturer's protocol.

Cells were harvested by trypsinization and washing with 1×DPBS 24 h after transfection. A 1/10th portion of the cells was saved for western blot analysis to confirm expression of Pitx2 and IKZF1. The remaining cells were lysed and processed using the Zymo-Duet kit with the IIICG column for both DNA and RNA without on-column DNase I treatment. DNA was eluted in 100 μL, and RNA was eluted in 80 μL and treated with DNase I (8 U)/ExoI (100 U)/ExoIII (100 U) for a minimum of 4 hr at 37° C. in a total reaction volume of 100 μL in 1× DNase I buffer. Assuming about 10 M cells per sample, an equivalent of about 10,000 cells gDNA and about 5000 cells nuclease-treated RNA was tested using QPCR with GFP as target to confirm the quality of transfection and completion of DNA removal in RNA, respectively. The reactions were spiked with another 2 U of DNase I as needed. RNA was column cleaned using a Zymo-IIIC column and eluted in 50 μL of water. An equivalent of about 4000 cells was used as a measure of quality control in a standard RT reaction as described in the genome-scale protocol. The remaining RNA was incubated with 80 pmole of GRAMc_RT_oligo (NJ-489) used for cDNA synthesis in an 80 μL 1× High-Capacity cDNA synthesis reaction using 8 μL of Multiscribe and 3.2 μL of dNTP but without the use of random primers for 4 hrs to overnight at 37° C. for a quality control QPCR following 2 hrs of RT. Upon completion of DNA digestion, 4 μL of NEBUFFER® 3 and 2 μL of RNase If were added to the reaction for 2 hr at 37° C. then spiked with Proteinase K for 15 min at 37° C. and heat inactivated for 10 min at 95° C. followed by overnight ethanol precipitation and resuspension in 30 μL of water.

N25 barcodes were preliminarily amplified as described above, but 6 cycles of a single 50 μL Q5® High-Fidelity DNA Polymerase reaction were used, and IX barcoding for IONTORRENT® Proton sequencing was used with the following primer pairs: for control-1: NJ-197/NJ523; for control-2: NJ-198/NJ523; for Pitx2-1: NJ-200/NJ523; for Pitx2-2: NJ-132/NJ523; for IKZF1-1: NJ-133/NJ523; and for IKZF1-2: NJ-134/NJ523. Data analysis was conducted as described above. The sequences of primers are available in Table 3.

Confirmation of ectopic Transcription Factor expression by Western Blot: An aliquot of each transfection condition (80 K library+CMV::pitx2, 80 K library+CMV::IKZF1, and 80 K library+CMV::EGFP) was lysed in 80 μL of RIPA buffer (150 mM NaCl, 1% NP40, 0.5% sodium deoxycholate, 0.1% SDS, 50 mM Tris-HCl pH 8.0, 5 mM EDTA) spiked with a 1:100 dilution of Halt Protease Inhibitor Cocktail (THERMOFISHER®) on ice for 30 min with intermittent flicking. Lysates were centrifuged at 12,000 RPM for 10 min at 4° C. and quantified using BCA reagent.

Approximately 25 ng of each sample was loaded in duplicate sets (expressed and control), separated on a 12% polyacrylamide gel, transferred to a PVDF membrane, and blotted with antibodies against FLAG (1:500, Santa Cruz sc-166355) or GAPDH (1:1000, Santa Cruz sc-25778). Horseradish peroxidase-conjugated secondary antibodies (1:5000) and enhanced chemiluminescence reagents (GE Healthcare) were used to detect bands on a Bio-Rad ChemiDoc MP system.

### Example 2

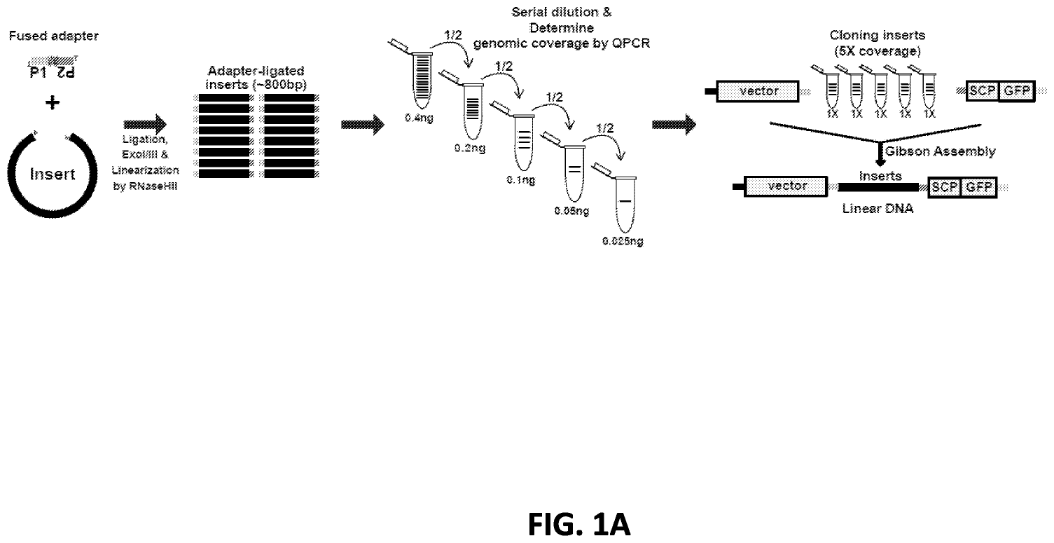

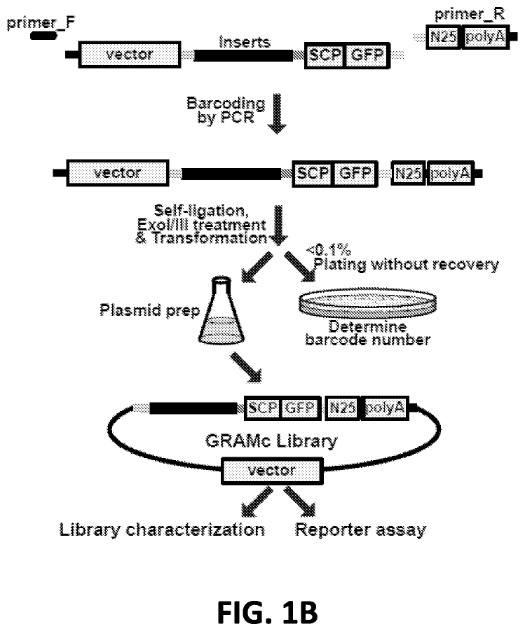

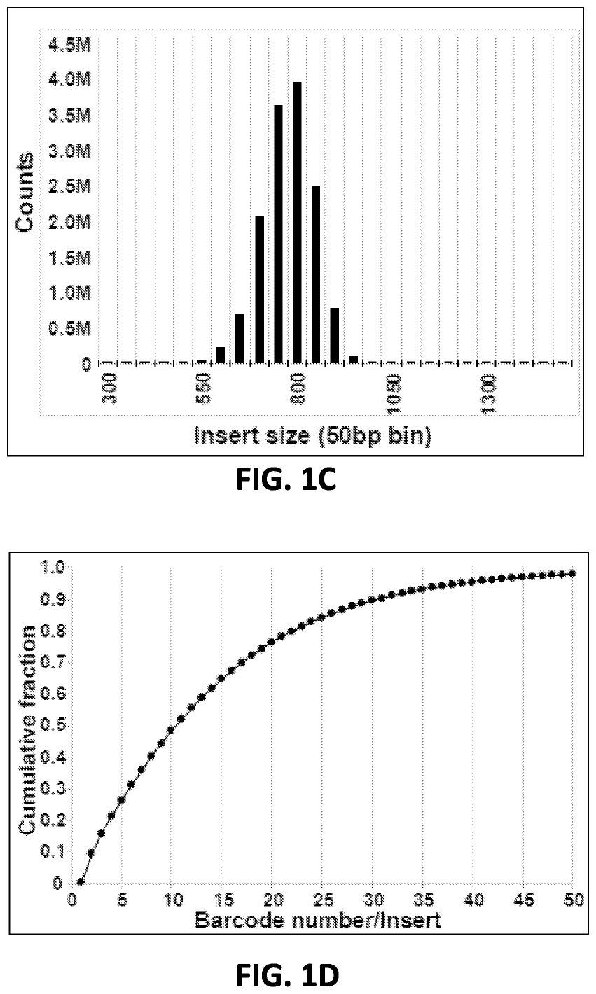

This example describes construction of a GRAMc library. In this example, a GRAMc library was generated by the following procedure (FIGS. 1A-1D). First, random genomic DNA fragments were size-selected, adapter-ligated, and serially diluted to reach an intended genomic coverage (FIG. 1A). To improve the accuracy of adapter ligation, an adapter (FIG. 6) was fused to form circular ligation products that can resist exonuclease I/III treatment against linear DNA, including non-ligated DNA and linear concatenates. After exonuclease treatment, circular ligation products were linearized by RNase HII, which cuts ribonucleotide sites (UU/AA) within the fused adapter. Linearized ligates were then serially diluted and PCR amplified using adapter-specific primers. A dilution of intended genomic coverage was identified by counting the presence or absence of 11 randomly chosen genomic regions by QPCR. For a dilution that contains about 4 M randomly sampled genomic DNA fragments ˜800 bp long (an average of 1× genomic coverage), the expected presence rate of target regions is 0.6. A dilution of 5× (or any desired genomic coverage) was assembled with two common pieces of DNA to form a library of linear DNA products that contain genomic test fragments, a basal promoter, a GFP ORF (Arnone, et al. Development 124.22 (1997): 4649-4659), and vector backbone (FIG. 7). The vector system uses a pan-bilaterian Super Core Promoter 1 (SCP) (Juven-Gershon, et al. Developmental biology 339.2 (2010): 225-229).

Second, the resulting genomic DNA library was barcoded with an excess number of random 25mers (N25) by PCR with a pair of common primers that can amplify the entire library including the vector backbone (FIG. 1B). One of the common primers, primer R, contains a random N25 in the middle and a core-poly adenylation signal (polyA) (Nag, et al. RNA 12.8 (2006): 1534-1544). The barcoded library was self-ligated, exonuclease I/III treated, and electroporated into E. coli for library amplification and plasmid extraction. A small fraction (e.g., 1/1,000th) of unrecovered transformants was used to measure the colony forming unit (cfu), and the remainder was used for library amplification in liquid culture and subsequent plasmid extraction. Because the PCR-mediated barcoding introduces an excess of barcodes, virtually all individual transformants contain unique barcodes. For example, barcodes present in transformants used for colony counting were not identified in the final library. The number of unique barcode reporters in a GRAMc library can be controlled by the scale of electroporation. In the protocol used herein, 4-10 ng of circular ligation products with inserts of about 800 bp consistently generated about 40 M cfu, which is comparable to the advertised efficiency of commercially available competent cells. The genomic coverage of the library that was determined in the first step is maintained as long as the number of unique barcodes harvested is much larger than the number of unique inserts. Purified plasmids were used for library characterization. Library characterization includes identification of genomic inserts and pairs of inserts and barcode reporters by ILLUMINA® paired-end sequencing (see Example 1 and FIG. 8).

Using the method, a human GRAMc library of inserts about 800 bp-long was generated. The intended numbers of unique genomic DNA inserts and unique barcodes in this library were 20 M (5× genomic coverage) and 200 M (10 barcodes/insert), respectively. After analyzing 479.1 M pairs of sequences mapped to the hg38 assembly (out of 519 M paired-end reads), 15.6 M genomic regions were identified. The total number of unique barcodes that were associated with these genomic regions was 191 M. The library covered 93.4% of the human genome at least once (Table 1).

TABLE 1
Genomic coverage of the human GRAMc library

| | | Coverage (unique, <95% |
|---|---|---|
| Chromosome | Coverage (all) | identity) |
| chr1 | 0.893 | 0.860 |
| chr2 | 0.962 | 0.948 |
| chr3 | 0.970 | 0.963 |
| chr4 | 0.968 | 0.960 |
| chr5 | 0.969 | 0.940 |
| chr6 | 0.963 | 0.954 |
| chr7 | 0.963 | 0.937 |
| chr8 | 0.966 | 0.951 |
| chr9 | 0.850 | 0.804 |
| chr10 | 0.963 | 0.948 |
| chr11 | 0.961 | 0.949 |
| chr12 | 0.964 | 0.959 |
| chr13 | 0.971 | 0.951 |
| chr14 | 0.968 | 0.939 |
| chr15 | 0.970 | 0.927 |
| chr16 | 0.867 | 0.820 |
| chr17 | 0.941 | 0.902 |
| chr18 | 0.969 | 0.941 |
| chr19 | 0.921 | 0.867 |
| chr20 | 0.956 | 0.936 |
| chr21 | 0.934 | 0.827 |
| chr22 | 0.937 | 0.861 |
| chrX | 0.866 | 0.828 |
| chrY | 0.423 | 0.300 |
| Genome | 0.934 | 0.907 |

Although obtaining more sequencing reads would improve these numbers, these numbers are already close to the intended numbers of inserts and barcodes in the library. Of the detected 15.6 M genomic regions, 13.8 M inserts were unique in sequences (<95% sequence identity with other genomic regions). In addition, the genomic distribution of unique inserts was more or less uniform (FIG. 2C). For unique inserts (FIG. 1C), 71% of the inserts were within 750-850 bp range, indicating that size selection was effective. Further, considering the number of barcodes per insert (FIG. 1D), although the barcode numbers of the majority of inserts deviated significantly from the expected number of 10, 99% and 55% of unique inserts were connected to ≥2 barcodes and ≥10 barcodes, respectively. Therefore, barcode-specific effects on reporter expression were insignificant in the GRAMc library. The list of genomic coordinates of inserts and their associated barcodes is available as FIG. 6.

### Example 3

In this example, the application of GRAMc in HepG2 cells is described. The GRAMc library was tested in two batches of 100 M HepG2 cells at the time of seeding or 200 M cells at the time of transfection. As a comparison, previous genome-scale enhancer screenings used 300 M LNCaP cells (Liu, et al. Genome biology 18.1 (2017): 219) and 800 M HeLa cells (Muerdter, et al. Nature methods 15.2 (2018): 141), and a genome-scale promoter screening used 100 M K562 cells (van Arensbergen, et al Nature biotechnology 35.2 (2017): 145). Following transfection of the GRAMc library into cells, total RNAs were extracted and reverse transcribed, and expressed barcodes were PCR amplified. To avoid losing reporter transcripts during secondary enrichment of mRNA (Muerdter, et al. Nature methods 15.2 (2018): 141) or reporter transcripts (Tewhey, et al. Cell 165.6 (2016): 1519-1529), the total RNAs and GRAMc-specific oligomers were used for reverse transcription. Expressed barcodes were amplified by PCR, and expression levels of reporters were measured by ILLUMINA® sequencing. A schematic of processing RNAs into sequencing libraries, along with the associated quality control steps is available in FIG. 9. Reporter expressions were double-normalized to the relative copy number of inserts in the input GRAMc library and background activities, which is the average activity of the middle 30% of rank ordered reporter expressions (Nam, et al. PNAS USA 107.8 (2010): 3930-3935). The background activity measured in this way has been very similar to the leaky activities of known inactive fragments in sea urchin embryos (Nam, et al. PNAS USA 107.8 (2010): 3930-3935, Guay, et al. Developmental biology 422.2 (2017): 92-104).

Approximately 200 M reads from each batch of expressed barcodes were obtained, and 78-79% of barcodes matched to barcodes with associated genomic regions. To account for copy number variations, approximately 450 M barcode reads were obtained from input plasmids. Because 99% of inserts are driving ≥2 barcodes, read numbers of multiple barcodes for the same insert were combined. Approximately 7.5 M inserts with ≥10 reads from input plasmids were used for data analysis. A total of 50,993 inserts from 41,216 non-overlapping genomic regions displayed activities of ≥5-fold higher than the background (bg) activity (red dots, ≥5×bg) in two independent experiments (FIG. 2A). The replicate GRAMc data showed a Pearson's correlation coefficient (r) of 0.95, and the probability of a CRM in one batch being considered a CRM in another batch was 0.80 (80% reproducibility of CRMs). When the cutoff was lowered to 3-fold of the background (orange and red dots, ≥3×bg), the number of active regions increased to 150,011 (62% reproducibility of CRMs).

To validate the accuracy of GRAMc, 11 CRMs (≥5×bg, red dots), 5 marginally active fragments (3-5×bg, orange dots), and 4 inactive fragments (≤1×bg, black dots) were randomly selected and their regulatory activities were individually tested with a one-by-one reporter assay (FIG. 2B). Levels of GFP transcripts relative to copies of transfected DNA were measured by QPCR. Reporter expressions were further normalized to the background activity (bg), which is the average level of the 4 inactive reporter constructs. Average levels of 4 independent assays are shown in black bars for individual inserts. Of the 11 CRMs tested, 8 inserts were ≥5×bg, while 2 inserts and 1 insert were 2.8×bg and 1.9×bg, respectively. This result is comparable to the 80% reproducibility of CRMs in GRAMc (FIG. 2A). In the case of the 5 marginally active inserts, 1 insert was 10×bg, 3 inserts were within the expected range of 3-5×bg, and 1 insert was 1.4×bg. Overall, cis-regulatory activities measured by GRAMc were reproducible in independent assays (R2=0.83). These results indicate that GRAMc is a reliable and efficient tool to discover CRMs at genome-scale.

### Example 4

This example describes GRAMc-identified CRMs that possess expected features of CRMs. As GRAMc is based on the standard configuration of reporter constructs, GRAMc-identified CRMs should possess known features of CRMs that have been identified by traditional reporter assays. First, CRMs should primarily be located near expressed genes in HepG2. The genomic locations of expressed genes in HepG2, CRMs, and the input library were compared, and the expressed genes and CRMs had similar patterns, while the input library was approximately uniformly distributed (FIGS. 2C and 10A-10F).

Second, CRMs are known to be enriched 5′-proximal to genes (promoters); however, the majority are located outside of the proximal regions (distal enhancers) (26). When the proportions of CRMs were computed for the number of inserts tested within sliding 2 kb windows upstream or downstream of expressed genes, the 5′-proximal 2 kb regions showed the highest enrichment (0.03) (FIG. 2D). The 3′-proximal 2 kb regions showed the second highest peaks, while genic regions are slightly depleted of CRMs. Despite these regional variations, CRMs are consistently enriched around expressed genes within at least 100 kb region in each direction compared to the genomic average of 0.0067. A similar pattern was also observed near unexpressed genes, but the degree of enrichment was lower than near expressed genes. These results indicate that GRAMc can efficiently identify both proximal promoters and distal enhancers.

Third, CRMs are expected to be associated with binding of transcription factors and other proteins that positively impact CRM function. The relative enrichment (total base pairs shared relative to random expectations) of narrow peaks was computed from 167 ENCODE ChIP-seq or DNase-seq data from HepG2 in CRMs versus inactive fragments (FIG. 2E), 153 data showed ≥2-fold enrichment in CRMs versus inactive regions. These include general transcriptional factors (e.g., GTF2F1, TAF1, and TBP), a transcriptional coactivator (P300), and histone modification enzymes (e.g., H3K4me3 and H3K9ac). ChIP-seq peaks that were not enriched or were even depleted in CRMs include transcription factors (TCF12 and BCLAF1), spliceosome components (PLRG1 and SNRNP70), and histone methylases (H3K27me3, H3K36me3 and H3K9me3). Interestingly, despite the overall enrichment, only 32% of GRAMc-identified CRMs overlapped with the 153 ENCODE data with ≥2-fold enrichment in CRMs, and 58% of CRMs did not overlap with any ENCODE data used in this analysis. Although obtaining ChIP-seq data for more transcription factors may increase the overlap, reporter assays may detect CRMs that are not active in the genome due to chromatin silencing or CRMs that can evade detection by ChIP-seq.

### Example 5

In this example, motif enrichment is shown to explain differential activities of ChromHMM predicted enhancers. Earlier studies have shown that, although CRM predictions based on chromatin marks are enriched in functionally validated CRMs, the majority of predicted CRMs do not drive significant expression in reporter assays (Liu, et al. (Genome biology 18.1 (2017): 219; Muerdter, et al. Nature methods 15.2 (2018): 141; van Arensbergen, et al. Nature biotechnology 35.2 (2017): 145). Consistent with these observations, in an assay of cis-regulatory activities of GRAMc-tested fragments that overlap ≥90% with ChromHMM-predicted strong enhancers in HepG2 (Ernst, et al. Nature methods 9.3 (2012): 215), approximately 80% of predicted enhancers showed ≤2-fold of the background activity in GRAMc (FIG. 3A). If the predicted enhancers are true enhancers, enrichment of transcription factor binding site (TFBS) motifs would be expected. Predicted strong enhancers were the focus herein because promoters are inherently enriched with motifs and predicted weak enhancers may increase ambiguity.

Enrichment of 601 HOCOMOCO_v10 HUMAN motifs (Kulakovskiy, et al. Nucleic acids research 44.D1 (2015): D116-D125) within predicted enhancers, GRAMc-identified CRMs, and inactive fragments were compared using FIMO software (Cuellar-Partida, et al. Bioinformatics 28.1 (2011): 56-62; Bailey, et al. Nucleic acids research 37 (2009): W202-W208). Overall, GRAMc-identified CRMs showed stronger enrichment of motifs than the predicted enhancers (FIG. 3B). The predicted enhancers that were active or marginally active in GRAMc (FIGS. 3C-3D) displayed enrichment or depletion of motifs comparable to that of the GRAMc-identified CRMs. On the contrary, enrichment of motifs gradually faded in predicted enhancers with weaker reporter expressions (FIGS. 3E-3G). Given their inability to drive significant reporter expressions and weak motif enrichment, it is likely that the majority of predicted enhancers are not true enhancers. However, this does not rule out the possibility that chromatin marks may indicate a neighborhood of enhancers rather than the exact location and that predicted enhancers may possess other types of cis-regulatory activities that cannot be measured in reporter assays.

Activation of the interferon pathway results in erroneous identification of interferon-responsive enhancers upon DNA transfection (Muerdter, et al Nature methods 15.2 (2018): 141), and such an artifact can reduce overlap between GRAMc-identified CRMs and ChromHMM predictions. However, consistent with the original discovery that HepG2 cells do not activate the pathway, motifs for interferon-stimulated transcription factors, including IRF1-9 and hMX1, were not enriched in GRAMc-identified CRMs.

### Example 6

This example shows that enriched motifs in CRMs predict a potentially new type of gene regulatory interaction. The pattern of reporter expression measured by small reporter constructs are direct readouts of the trans-regulatory environment in host cells. Because the DNA sequences of CRMs contain binding sites for transcription factors, computational motif analysis has often been used to infer gene regulatory programs (e.g., Xie, et al. Nature 434.7031 (2005): 338; Marini, et al. Cell systems 5.3 (2017): 187-201; Enuameh, et al Genome research (2013): gr-151472; Markstein, et al. Development 131.10 (2004): 2387-2394; Halfon, et al. BMC genomics 12.1 (2011): 578). Based on 601 HOCOMOCO_v10 HUMAN motifs (Kulakovskiy, et al. Nucleic acids research 44.D1 (2015): D116-D125) computationally predicted in the CRMs and in inactive fragments (negative controls) by FIMO, abundance (the proportion of motif− positive CRMs or inactive fragments) and relative enrichment of motifs (relative abundance of a motif in CRMs versus inactive fragments) were computed (FIG. 4A). The results show that 176 out of 601 motifs were ≥2-fold enriched in CRMs compared with inactive fragments. While the majority (65%) of enriched motifs were for expressed (FPKM≥1) transcription factors, interestingly, the remainder were for transcription factors that are either not expressed or have very low expression (FPKM<1) (3).

Enriched motifs for expressed transcription factors should predict positive regulators for the CRMs identified in HepG2. To assay for regulators, the motif analysis results were compared with ENCODE ChIP-seq data from HepG2 cells (3). If a predicted transcription factor based on motif enrichment is correct, ChIP-seq peaks for the same transcription factor should also be enriched. A total of 58 transcription factors were common between the two datasets. Of the 58 factors, 31 motifs and 56 ChIP-seq peaks were enriched ≥2-fold in CRMs versus inactive fragments (FIG. 4B). Given that all but one of the enriched motifs were also enriched in ChIP-seq data, prediction of positive regulators based on motif enrichment has a very low false-positive rate (<<0.1). The other approximately 50% of transcription factors showed <2-fold enrichment of motifs, but the ChIP-seq peaks were still highly enriched. Although more detailed analyses are necessary, in a conservative scenario, the motif-based prediction herein exhibits a false negative rate of about 0.5.

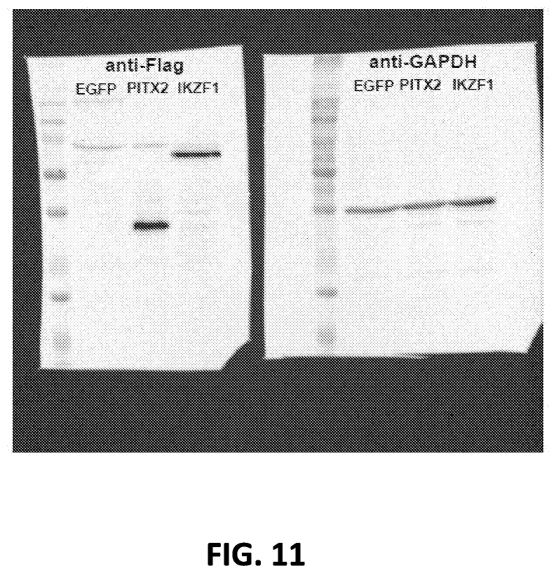

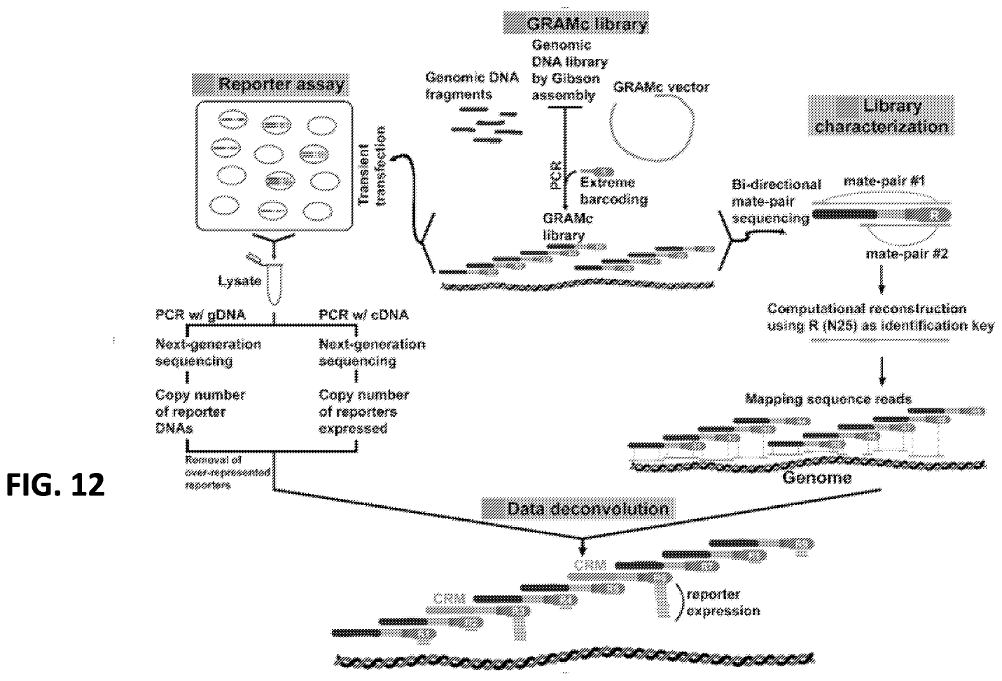

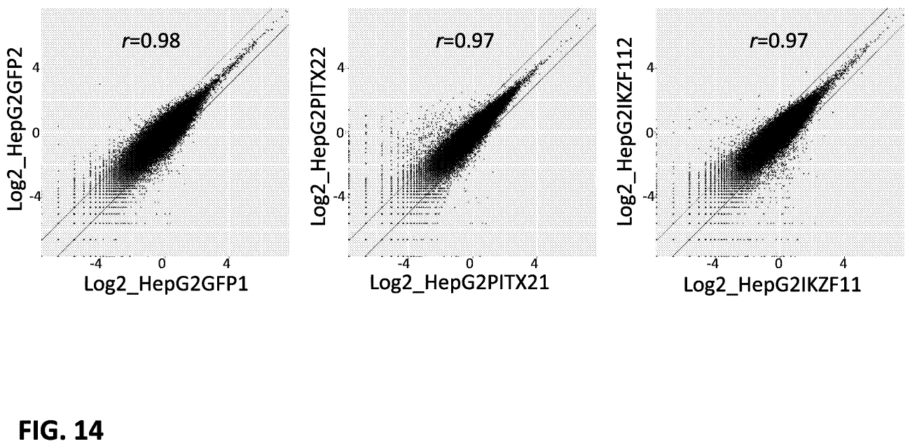

Enrichment of motifs for nonexpressed transcription factors indicates that they control the HepG2-CRMs either as an activator or as a repressor in other cell types or conditions (FIG. 4C). Ectopic expression of candidate transcription factors in HepG2 was used to assay for such regulators. Two transcription factor genes, pitx2 (a homeobox gene) and ikzf1 (an ikaros homolog), were examined. In mice, pitx2 is expressed in and is required for hematopoietic function of the fetal liver, and shut down of both pitx2 and hematopoietic function of the fetal liver is essential for differentiation of the adult liver from the fetal liver (Kieusseian, et al. Blood 107.2 (2006): 492-500). Similarly, ikzf1 is a key regulator of hematopoietic development (Davis. Therapeutic advances in hematology 2.6 (2011). 359-368) and is expressed in the fetal liver (Roy, et al PNAS USA (2012): 201211405); although its function in hepatic development is not known. Plasmids that can constitutively express mRNAs of pitx2 (CMV::pitx2) or ikzf1 (CMV::ikzf1) were co-transfected with a set of randomly selected about 80,000 GRAMc reporter constructs from the full GRAMc library. As a control experiment, plasmids that can constitutively express GFP mRNAs (CMV::gfp) were co-transfected with the same set of reporter constructs. Replicate experiments of all three experiments were highly reproducible (Pearson's r≥0.99) (FIG. 14). Ectopic expression of pitx2 in HepG2 down-regulated the majority of CRMs by ≥2-fold, and this down-regulation was more pronounced in pitx2 motif− positive CRMs (Two-Sample t-test, P=4.4E-16) (FIG. 4D). In the case of ikzf1, only 9 CRMs were downregulated by ≥2-fold, and 6 of the 9 down-regulated CRMs were positive for IKZF1 motif (Two-Sample t-test, P=2.5E-4) (FIG. 4E). Protein expression of both recombinant genes was confirmed by western blot (FIG. 11). These results indicate that pitx2 (and ikzf1, to a minor degree) maintains HepG2-CRM repression in the fetal liver, and clearance of pitx2 is critical for activation of HepG2-CRMs and gene expression in the adult liver. These results indicate that CRMs are not only useful for predicting regulatory programs in the host cell but also for predicting regulatory interactions between cells separated in time and space.

### Example 7

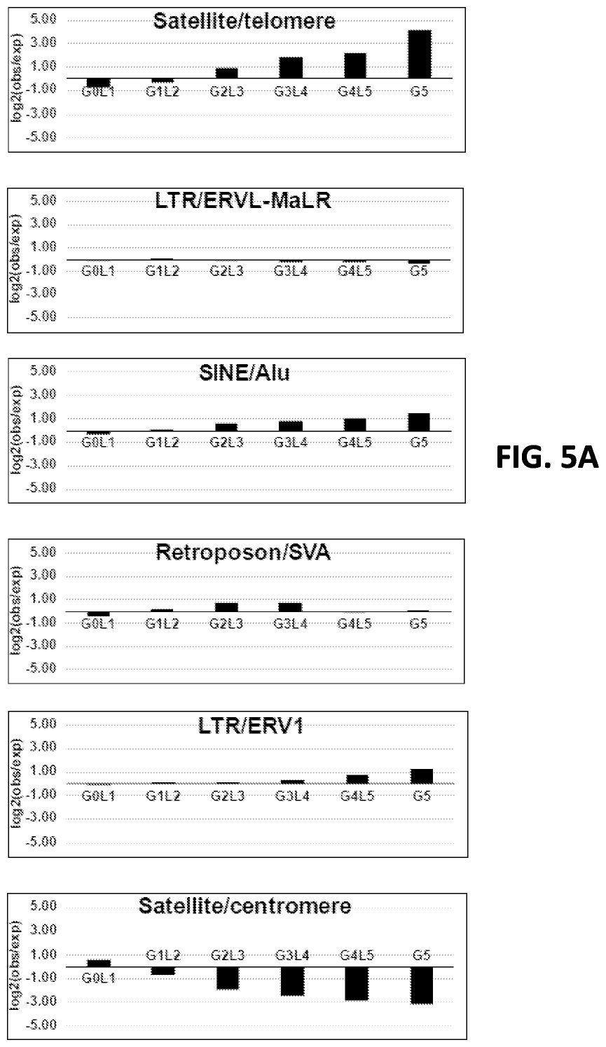

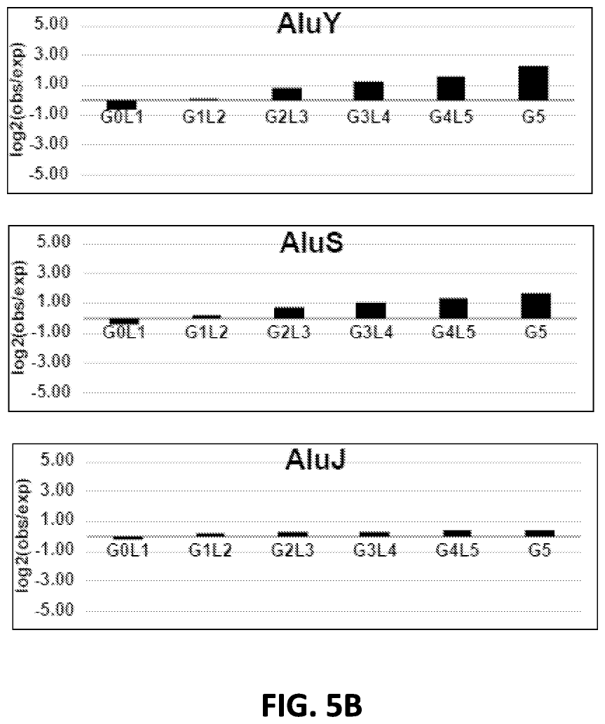

This example shows that SINE/Alu elements are enriched in CRMs. Early models for eukaryotic gene regulation proposed that repeat elements were a key player of gene expression control (McClintock. PNAS USA 36.6 (1950): 344-355; Britten, et al. Science 165.3891 (1969): 349-357). These predictions were later supported by multiple examples of Alu and ERV elements contributing to gene regulation and its evolution (Britten. PNAS USA 93.18 (1996): 9374-9377). Further, genomic surveys of chromatin signatures have shown that SINE/Alu elements are enriched in putative CRMs (Su, et al. Cell reports 7.2 (2014): 376-385; Trizzino, et al. BMC genomics 19.1 (2018): 468). However, genome-scale reporter assays for enhancers (Muerdter, et al. Nature methods 15.2 (2018): 141) or promoters (van Arensbergen, et al. Nature biotechnology 35.2 (2017): 145) have detected enrichment of LTR/ERV1 and LTR/ERVL-MaLR in CRMs but not SINE/Alu. To assay for such enrichment in GRAMc-identified CRMs, the data herein were compared with annotated repeat elements in the human genome (Smit, et al. “RepeatMasker Open-4.0” (2015)). Three families of repeat elements were detected, satellite/telomere, SINE/Alu and LTR/ERV1, as enriched ≥2-fold in CRMs (G5 set in FIG. 5A); however, LTR/ERVL-MaLR was not enriched in CRMs. The three elements were also enriched in marginally active G3L4 and G4L5 sets to lesser degrees. Interestingly, alpha-satellites were depleted by about 8-fold in CRMs, indicating a repressive function or incompatibility with other CRMs in HepG2. However, depletion of retroposon/SVA elements predicted to be transcriptional repressors in liver were not detected (Trizzino. Genome research 27.10 (2017): 1623-1633).

Using the GRAMc-identified CRMs, the evolution of Alu elements toward enhancers as a function of time was assayed (Su, et al. Cell reports 7.2 (2014): 376-385). Enrichment of Alu elements in CRMs should positively correlate with age. However, three major subfamilies of Alu (FIG. 5B) were examined, and the youngest subfamily (AluY) and the intermediate subfamily (AluS) showed ≥3-fold enrichment in CRMs, while the oldest subfamily (AluJ) showed only moderate enrichment (1.3-fold). Because the original study is based on the chromatin annotations in HeLa cells, this discrepancy can be explained by differences in cell types. Thus, subfamilies of 19 Alu elements that were tested with luciferase assays in HeLa cells were compiled (Su, et al. Cell reports 7.2 (2014): 376-385). Consistent with these results, 8/10 AluY or AluS elements were active, and only 4/9 AluJ elements were active. Therefore, the results are consistent with an alternative model that Alu elements lose regulatory activity with age.

These results demonstrate that GRAMc data can be useful for testing multiple evolutionary genomics hypotheses and that it can lead to different conclusions compared to the data generated by earlier genome-scale reporter assays or chromatin annotations. Further, it is possible that the observed discrepancies between GRAMc and earlier reporter assays can be attributed in large part to different cell types used. Enrichment of the entire list of repeat elements is available in Table 2.

TABLE 2
Enrichment of the entire list of repeat elements.

| Repeat | Total | G5 | G4L5 | G3L4 | G2L3 | G1L2 | L1 |
|---|---|---|---|---|---|---|---|
| Satellite/telomere | 207 | 4.26 | 2.23 | 1.89 | 1.02 | −0.24 | −0.64 |
| DNA/Merlin | 130 | 2.08 | Null | 0.39 | 0.11 | −0.04 | −0.04 |
| SINE/Alu | 1966506 | 1.44 | 1.06 | 0.80 | 0.59 | 0.14 | −0.35 |
| LTR/ERV1 | 407424 | 1.28 | 0.73 | 0.38 | 0.14 | 0.01 | −0.08 |
| DNA/PIF-Harbinger | 57 | 1.26 | Null | Null | 0.62 | 0.24 | −0.37 |
| LTR/ERVK | 28785 | 0.61 | 0.82 | 0.61 | 0.40 | 0.08 | −0.19 |
| DNA/TcMar-Tc1 | 1596 | 0.36 | −1.13 | −0.64 | −0.34 | 0.00 | 0.05 |
| LINE/Dong-R4 | 1656 | 0.31 | −1.77 | −1.11 | −0.66 | −0.02 | 0.10 |
| DNA?/PiggyBac? | 877 | 0.13 | −0.27 | −0.19 | −0.22 | 0.04 | −0.01 |
| Retroposon/SVA | 9557 | 0.10 | −0.05 | 0.71 | 0.79 | 0.24 | −0.46 |
| DNA?/hAT-Tip100? | 1324 | −0.10 | −0.12 | 0.29 | −0.31 | −0.03 | 0.05 |
| LINE/L1 | 2284142 | −0.12 | 0.16 | 0.31 | 0.21 | 0.00 | −0.03 |
| DNA/hAT-Tag1 | 6242 | −0.15 | −0.78 | −0.61 | −0.28 | −0.02 | 0.07 |
| DNA/PiggyBac | 5676 | −0.16 | −0.38 | −0.04 | 0.11 | 0.05 | −0.06 |
| LTR/Gypsy? | 19209 | −0.17 | −0.66 | −0.61 | −0.39 | −0.07 | 0.12 |
| DNA/TcMar-Tigger | 335319 | −0.20 | −0.35 | −0.35 | −0.27 | −0.03 | 0.06 |
| DNA/hAT-Charlie | 600484 | −0.26 | −0.34 | −0.33 | −0.19 | 0.01 | 0.03 |
| SINE/MIR | 1297906 | −0.26 | −0.26 | −0.23 | −0.07 | 0.05 | −0.03 |
| DNA/TcMar-Mariner | 43263 | −0.27 | −0.36 | −0.33 | −0.22 | 0.01 | 0.03 |
| DNA/TcMar-Tc2 | 20434 | −0.30 | −0.53 | −0.55 | −0.21 | −0.02 | 0.06 |
| LTR/ERVL-MaLR | 854714 | −0.33 | −0.21 | −0.18 | −0.07 | 0.03 | −0.01 |
| DNA/hAT | 20960 | −0.34 | −0.36 | −0.34 | −0.07 | 0.07 | −0.05 |
| DNA/MULE-MuDR | 4365 | −0.35 | 0.16 | −0.12 | −0.29 | −0.08 | 0.11 |
| DNA/hAT-Tip100 | 124069 | −0.35 | −0.43 | −0.46 | −0.19 | 0.00 | 0.04 |
| LINE/L2 | 1149160 | −0.41 | −0.46 | −0.44 | −0.30 | −0.02 | 0.07 |
| DNA/hAT-Tip100? | 6010 | −0.41 | −0.82 | −0.82 | −0.29 | −0.03 | 0.08 |
| LTR | 22195 | −0.42 | −0.63 | −0.45 | −0.46 | −0.06 | 0.12 |
| RC/Helitron | 4441 | −0.43 | −0.49 | −0.80 | −0.45 | −0.01 | 0.08 |
| LTR? | 22607 | −0.45 | −0.39 | −0.54 | −0.30 | −0.02 | 0.07 |
| LINE/RTE-X | 43695 | −0.46 | −0.72 | −0.60 | −0.38 | −0.03 | 0.09 |
| DNA/hAT-Blackjack | 49402 | −0.46 | −0.14 | −0.34 | −0.20 | 0.03 | 0.01 |
| Low_complexity | 184214 | −0.47 | −0.36 | −0.35 | −0.17 | 0.04 | 0.00 |
| Satellite | 13403 | −0.48 | 1.09 | 1.26 | 1.72 | 0.18 | −0.84 |
| LTR/ERVL | 396490 | −0.50 | −0.46 | −0.44 | −0.31 | −0.04 | 0.08 |
| LINE/RTE-BovB | 24224 | −0.55 | −0.64 | −0.58 | −0.48 | −0.08 | 0.14 |
| LTR/ERVL? | 10340 | −0.59 | −0.50 | −0.46 | −0.32 | −0.04 | 0.09 |
| LINE/CR1 | 171516 | −0.62 | −0.60 | −0.65 | −0.39 | −0.03 | 0.09 |
| LTR/Gypsy | 45290 | −0.64 | −0.31 | −0.76 | −0.43 | −0.05 | 0.11 |
| LINE/Penelope | 3005 | −0.65 | −0.30 | −0.33 | −0.49 | −0.04 | 0.10 |
| LTR/ERV1? | 4173 | −0.68 | −1.10 | −0.37 | −0.47 | −0.07 | 0.13 |
| DNA? | 10094 | −0.85 | −0.57 | −1.19 | −0.56 | −0.03 | 0.11 |
| SINE/5S-Deu-L2 | 7226 | −0.86 | −0.72 | −0.82 | −0.30 | −0.03 | 0.09 |
| DNA/hAT-Ac | 13759 | −0.87 | −0.65 | −0.44 | −0.46 | −0.03 | 0.10 |
| RC?/Helitron? | 1315 | −0.94 | −0.11 | −0.14 | −0.16 | 0.01 | 0.02 |
| Unknown | 18054 | −0.98 | −0.89 | −0.67 | −0.47 | −0.05 | 0.12 |
| DNA/TcMar | 5016 | −1.02 | −1.20 | −1.12 | −0.27 | −0.03 | 0.09 |
| DNA | 18278 | −1.04 | −1.03 | −0.62 | −0.49 | −0.02 | 0.10 |
| DNA/hAT? | 6228 | −1.05 | −0.43 | −1.06 | −0.61 | −0.08 | 0.16 |
| None | 946660 | −1.49 | −0.85 | −0.82 | −0.54 | −0.07 | 0.15 |
| DNA/Kolobok | 775 | −1.50 | Null | −0.86 | −1.38 | −0.01 | 0.15 |
| LINE/L1-Tx1 | 488 | −1.83 | Null | −0.19 | −0.71 | −0.06 | 0.15 |
| RNA | 1951 | −1.83 | −1.00 | −1.06 | −0.21 | −0.07 | 0.12 |
| DNA/TcMar? | 522 | −1.93 | −0.10 | −0.62 | −0.73 | 0.09 | 0.01 |
| LINE/Jockey | 584 | −2.09 | −1.27 | −0.45 | −0.60 | −0.14 | 0.20 |
| Satellite/centromere | 54022 | −3.23 | −2.93 | −2.43 | −1.92 | −0.72 | 0.58 |
| DNA/TcMar-Pogo | 99 | Null | Null | Null | −0.18 | −0.10 | 0.17 |
| Satellite/acro | 76 | Null | Null | 0.17 | −0.12 | 0.35 | −0.37 |

Note:
Enrichment scores are in log2 scale.

In view of the many possible embodiments to which the principles of the disclosure may be applied, it should be recognized that the illustrated embodiments are only examples and should not be taken as limiting the scope of the invention. Rather, the scope of the invention is defined by the following claims. We therefore claim as our invention all that comes within the scope and spirit of these claims.

1. A method of constructing a nucleic acid molecule reporter library, comprising:
isolating a plurality of nucleic acid molecules of a selected size range;
ligating the plurality of isolated nucleic acid molecules of the selected size range to at least one linear adapter sequence using a ligase, wherein the linear adapter sequence comprises at least two consecutive ribonucleotides flanked by at least one deoxyribonucleotide on the 3′ end, and at least one deoxyribonucleotide on the 5′ end, thereby producing a plurality of circular nucleic acid molecules comprising an insert and an adapter;
contacting the plurality of circular nucleic acid molecules comprising an insert and an adapter with an exonuclease under conditions sufficient to remove linear nucleic acid molecules from the plurality of circular nucleic acid molecules;
contacting the plurality of circular nucleic acid molecules comprising an insert and an adapter with an endoribonuclease under conditions sufficient to produce a plurality of linear nucleic acid molecules each comprising the at least one deoxyribonucleotide on the 3′ end and the at least one deoxyribonucleotide on the 5′ end, flanking the insert; and
fusing each of the plurality of linear nucleic acid molecules to at least one reporter nucleic acid to produce a plurality of reporter constructs, thereby producing the nucleic acid molecule reporter library.

2-3. (canceled)

4. The method of claim 1, wherein the plurality of nucleic acid molecules of a selected size range are about 100-3000 base pairs long or are about 750-850 base pairs long.

5. (canceled)

6. The method of claim 1, wherein the plurality of isolated nucleic acid molecules of a selected size range are selected using gel electrophoresis or bead-based size selection.

7. The method of claim 1, wherein the plurality of nucleic acid molecules of a selected size range comprise genomic DNA or synthetic DNA.

8. The method of claim 7, wherein the genomic DNA is from a mammalian cell, plant cell, bacterial cell, fungal cell, or archaeal cell.

9-14. (canceled)

15. The method of claim 1, wherein contacting the plurality of circular nucleic acid molecules comprising an insert and an adapter with the endoribonuclease comprises contacting the plurality of circular nucleic acid molecules comprising an insert and an adapter with an endoribonuclease specific for ribonucleotides within a DNA duplex.

16. (canceled)

17. The method of claim 1, further comprising determining genomic coverage of the plurality of linear nucleic acid molecules comprising the at least one deoxyribonucleotide on the 3′ end and the at least one deoxyribonucleotide on the 5′ end, flanking the insert.

18. The method of claim 17, wherein the determining genomic coverage comprises:
selecting at least one genomic region of interest;
amplifying the plurality of linear nucleic acid molecules comprising the at least one deoxyribonucleotide on the 3′ end and the at least one deoxyribonucleotide on the 5′ end, flanking the insert; and
determining the whether the selected genomic region is present in the plurality of linear nucleic acid molecules.

19. The method of claim 1, wherein the at least one reporter nucleic acid comprises a nucleic acid encoding a fluorescent protein and/or comprising a barcode nucleic acid.

20. The method of claim 1, further comprising fusing the plurality of linear nucleic acid molecules comprising the at least one deoxyribonucleotide on the 3′ end and the at least one deoxyribonucleotide on the 5′ end, flanking the insert, to a linear vector nucleic acid, thereby producing a plurality of linear vectors.

21-22. (canceled)

23. The method of claim 20, further comprising:
contacting each of the plurality of linear vectors with a primer nucleic acid comprising a barcode reporter construct;
performing a polymerase chain reaction (PCR), thereby producing a plurality of amplified vectors comprising the barcode reporter construct;
ligating the amplified vectors comprising the barcode reporter construct, thereby producing a plurality of circular vectors comprising the barcode reporter construct; and
contacting the plurality of circular vectors comprising the barcode reporter construct with an exonuclease under conditions sufficient to remove linear nucleic acid molecules from the plurality of circular vectors comprising the barcode reporter construct.

24. A method of constructing a nucleic acid molecule reporter library, comprising:
(i) isolating a plurality of nucleic acid molecules of a selected size range;
ligating the plurality of isolated nucleic acid molecules of the selected size range to at least one linear adapter sequence using a ligase, wherein the linear adapter sequence comprises at least two consecutive ribonucleotides flanked by at least one deoxyribonucleotide on the 3′ end, and at least one deoxyribonucleotide on the 5′ end, thereby producing a plurality of circular nucleic acid molecules comprising an insert and an adapter;
(ii) contacting the plurality of circular nucleic acid molecules comprising an insert and an adapter with an exonuclease under conditions sufficient to remove linear nucleic acid molecules from the plurality of circular nucleic acid molecules;
(iii) contacting the plurality of circular nucleic acid molecules comprising an insert and an adapter with an endoribonuclease under conditions sufficient to produce a plurality of linear nucleic acid molecules each comprising the at least one deoxyribonucleotide on the 3′ end and the at least one deoxyribonucleotide on the 5′ end, flanking the insert;
(iv) determining genomic coverage of the plurality of linear nucleic acid molecules comprising the at least one deoxyribonucleotide on the 3′ end and the at least one deoxyribonucleotide on the 5′ end, flanking the insert, comprising:
(a) selecting at least one genomic region of interest,
(b) amplifying the plurality of linear nucleic acid molecules comprising the at least one deoxyribonucleotide on the 3′ end and the at least one deoxyribonucleotide on the 5′ end, flanking the insert, and
(c) determining the whether the selected genomic region is present in the plurality of linear nucleic acid molecules; and
(v) fusing the plurality of linear nucleic acid molecules comprising the at least one deoxyribonucleotide on the 3′ end and the at least one deoxyribonucleotide on the 5′ end, flanking the insert to at least one reporter nucleic acid to produce a plurality of reporter constructs, comprising:
(a) fusing the plurality of linear nucleic acid molecules comprising the at least one deoxyribonucleotide on the 3′ end and the at least one deoxyribonucleotide on the 5′ end, flanking the insert to a linear vector nucleic acid, thereby producing a plurality of linear vectors;
(b) contacting each of the plurality of linear vectors with a primer comprising a barcode nucleic acid; and
(c) performing a polymerase chain reaction (PCR), producing a plurality of circular vectors comprising a barcode reporter construct comprising the at least one deoxyribonucleotide on the 3′ end and the at least one deoxyribonucleotide on the 5′ end, flanking the insert and a barcode; and
(d) contacting the plurality of circular vectors comprising the barcode reporter construct with an exonuclease under conditions sufficient to remove linear nucleic acid molecules from the plurality of circular vectors comprising a barcode reporter construct.

25-27. (canceled)

28. A nucleic acid molecule reporter library produced using the method of claim 1.

29. A method of detecting functional nucleic acid regulatory elements, comprising:
wherein the plurality of nucleic acid molecules comprise at least 80% of the cis regulatory elements in a selected genome of interest
transfecting at least one cell of interest with the library of claim 28; and
measuring the at least one reporter.

30. The method of claim 29, further comprising identifying and/or quantifying the at least one reporter.

31. The method of claim 29, further comprising isolating RNA from the cell of interest, producing isolated RNA.

32. The method of claim 31, wherein measuring the reporter comprises:
reverse transcribing the isolated RNA, producing cDNA; and
detecting the cDNA.

33-34. (canceled)

35. The method of claim 29, wherein the at least one reporter is at least one unique barcode nucleic acid.

36. The method of claim 35, wherein detecting the cDNA comprises:
amplifying the cDNA; and
identifying the at least one unique nucleic acid barcode.

37. The method claim 36, wherein amplifying the cDNA and identifying the at least one unique nucleic acid barcode comprises:
selecting primers specific for nucleotides comprising at least one unique nucleic acid barcode;
contacting the primers with the cDNA;
performing PCR using the primers and the cDNA, producing amplified DNA; and
sequencing the amplified DNA.

38. (canceled)

39. The method of claim 35, further comprising quantifying the at least one unique nucleic acid barcode.

40. The method of claim 29, wherein the at least one cell is a mammalian cell, plant cell, fungal cell, bacterial cell, or archaeal cell.

41-48. (canceled)

49. The method of claim 1, wherein the plurality of nucleic acid molecules comprise at least 80% of a selected genome of interest or wherein the plurality of nucleic acid molecules comprise at least 80% of the cis regulatory elements in a selected genome of interest.

50-56. (canceled)


SEQUENCE LISTING (124 sequences)

SEQ ID NO: 1 - DNA, 52 bp, Artificial Sequence
  Example linear adaptor sequence
ctgctgaatcactagtgaattattacccuucaagacactactctccagcagt

SEQ ID NO: 2 - DNA, 52 bp, Artificial Sequence
  Example linear adaptor sequence
  misc_RNA (24)..(25)
ctgctggagagtagtgtcttgaagggtaataattcactagtgattcagcagt

SEQ ID NO: 3 - DNA, 41 bp, Artificial Sequence
  Example primer sequence, NJ-95, Gibson_SCP1_amp1
ctgctggagagtagtgtcttgtacttatataagggggtggg

SEQ ID NO: 4 - DNA, 26 bp, Artificial Sequence
  Example primer sequence, NJ-96, EcoP15l_P1r_lin
ctgctgaatcactagtgaattcgcgg

SEQ ID NO: 5 - DNA, 19 bp, Artificial Sequence
  Example primer sequence, NJ-101, 3PofN25merShort
ggcgcgccgctgagggagt

SEQ ID NO: 6 - DNA, 23 bp, Artificial Sequence
  Example primer sequence, NJ-126, NT_del_F
aattcgccctatagtgagtcgta

SEQ ID NO: 7 - DNA, 71 bp, Artificial Sequence
  Example primer sequence, NJ-127, bN25_polyA_R-1/primer_R
  misc_feature (1)..(1): 5′ biotin modification
  misc_feature (22)..(46): n is a, c, g, t or u
tacagtccgacgatccagcagnnnnnnnnnnnnnnnnnnnnnnnnnggcgcgccgctgagggagtctagag

SEQ ID NO: 8 - DNA, 66 bp, Artificial Sequence
  Example primer sequence, NJ-128, pN25_polyA_R-2
  misc_feature (1)..(1): 5′ phosphorylation modification
cacaaaccacaactagaatgcagtgaaaaaaatgctttatttgtttacagtccgacgatccagcag

SEQ ID NO: 9 - DNA, 23 bp, Artificial Sequence
  Example sequence listing, NJ-129, pNT_del_F
aattcgccctatagtgagtcgta

SEQ ID NO: 10 - DNA, 64 bp, Artificial Sequence
  Example primer sequence, NJ-132, GRAMc_Ion-A_IX7_P4s
ccatctcatccctgcgtgtctccgactcagttcgtgattcgattacagtccgacgatccagcag

SEQ ID NO: 11 - DNA, 64 bp, Artificial Sequence
  Example primer sequence, NJ-133, GRAMc_Ion-A_IX8_P4s
ccatctcatccctgcgtgtctccgactcagttccgataacgattacagtccgacgatccagcag

SEQ ID NO: 12 - DNA, 64 bp, Artificial Sequence
  Example primer sequence, NJ-134, GRAMc_Ion-A_IX9_P4s
ccatctcatccctgcgtgtctccgactcagtgagcggaacgattacagtccgacgatccagcag

SEQ ID NO: 13 - DNA, 17 bp, Artificial Sequence
  Example primer sequence, NJ-141, pGRAMc_nP3_short
  misc_feature (1)..(1): Phosphorylation modification
tagactccctcagcggc

SEQ ID NO: 14 - DNA, 21 bp, Artificial Sequence
  Example primer sequence, NJ-142, pGRAMc_P4_short
  misc_feature (1)..(1): Phosphorylation modification
tacagtccgacgatccagcag

SEQ ID NO: 15 - DNA, 19 bp, Artificial Sequence
  Example primer sequence, NJ-145, S_3PofN25merShort
  misc_feature (1)..(7): Nucleotide bonds are phosphorothioated bonds from nucleotides 1-2 through 6-7
ggcgcgccgctgagggagt

SEQ ID NO: 16 - DNA, 23 bp, Artificial Sequence
  Example primer sequence, NJ-146, S_NT_del_F
  misc_feature (1)..(7): Nucleotide bonds are phosphorothioated bonds from nucleotides 1-2 through 6-7
aattcgccctatagtgagtcgta

SEQ ID NO: 17 - DNA, 59 bp, Artificial Sequence
  Example primer sequence, NJ-179, CRSP_F_T7_backbone
ttaatacgactcactataggtcgtagttatctacacgacggttttagagctagaaatag

SEQ ID NO: 18 - DNA, 59 bp, Artificial Sequence
  Example primer sequence, NJ-180, CRSP_F_T7_GFP
ttaatacgactcactataggcgcgctgaagtcaagttcgagttttagagctagaaatag

SEQ ID NO: 19 - DNA, 20 bp, Artificial Sequence
  Example primer sequence, NJ-183, CRSP_R
aaaagcaccgactcggtgcc

SEQ ID NO: 20 - DNA, 64 bp, Artificial Sequence
  Example primer sequence, NJ-197, GRAMc_Ion-A_IX1_P4s
ccatctcatccctgcgtgtctccgactcagctaaggtaacgattacagtccgacgatccagcag

SEQ ID NO: 21 - DNA, 64 bp, Artificial Sequence
  Example primer sequence, NJ-198, GRAMc_Ion-A_IX2_P4s
ccatctcatccctgcgtgtctccgactcagtaaggagaacgattacagtccgacgatccagcag

SEQ ID NO: 22 - DNA, 64 bp, Artificial Sequence
  Example primer sequence, NJ-200, GRAMc_Ion-A_IX3_P4s
ccatctcatccctgcgtgtctccgactcagaagaggattcgattacagtccgacgatccagcag

SEQ ID NO: 23 - DNA, 20 bp, Artificial Sequence
  Example primer sequence, NJ-208, pGRAMc_P1s_NoT
  misc_feature (1)..(1): 5′ phosphorylation modification
attcactagtgattcagcag

SEQ ID NO: 24 - DNA, 18 bp, Artificial Sequence
  Example primer sequence, NJ-209, pGRAMc_P2s_NoT
  misc_feature (1)..(1): 5′ phosphorylation modification
gacactactctccagcag

SEQ ID NO: 25 - DNA, 25 bp, Artificial Sequence
  Example primer sequence, NJ-213, Gibson_P1-T
gcgaattcactagtgattcagcagt

SEQ ID NO: 26 - DNA, 22 bp, Artificial Sequence
  Example primer sequence, NJ-214, Gibson_iNBP-T
caagacactactctccagcagt

SEQ ID NO: 27 - DNA, 20 bp, Artificial Sequence
  Example primer sequence, NJ-268, Hs-Top1_QF
acttcgtgtggagcacatca

SEQ ID NO: 28 - DNA, 20 bp, Artificial Sequence
  Example primer sequence, NJ-269, Hs-Top1_QR
cgtttctcaacagggacctt

SEQ ID NO: 29 - DNA, 20 bp, Artificial Sequence
  Example primer sequence, NJ-270, Hs-ACTA1_QF
atggtcggtatgggtcagaa

SEQ ID NO: 30 - DNA, 20 bp, Artificial Sequence
  Example primer sequence, NJ-271, Hs-ACTA1_QR
tctccatgtcatcccagttg

SEQ ID NO: 31 - DNA, 19 bp, Artificial Sequence
  Example primer sequence, NJ-276, Hs-AXL_QF2
ctgtcagacgatgggatgg

SEQ ID NO: 32 - DNA, 20 bp, Artificial Sequence
  Example primer sequence, NJ-277, Hs-AXL_QR2
taaggggtgtgaggatggag

SEQ ID NO: 33 - DNA, 20 bp, Artificial Sequence
  Example primer sequence, NJ-278, Hs-DLX5_QF
tacacaagtgcagccagctc

SEQ ID NO: 34 - DNA, 22 bp, Artificial Sequence
  Example primer sequence, NJ-279, Hs-DLX5_QR
gagtaagagagagcagcccatc

SEQ ID NO: 35 - DNA, 20 bp, Artificial Sequence
  Example primer sequence, NJ-280, Hs-NOTCH2_QF
aaatgcctcacaggcttcac

SEQ ID NO: 36 - DNA, 20 bp, Artificial Sequence
  Example primer sequence, NJ-281, Hs-NOTCH2_QR
cactggcactggtaggaacc

SEQ ID NO: 37 - DNA, 20 bp, Artificial Sequence
  Example primer sequence, NJ-282, Hs-RPP30_QF
ctgcttccaggagacctgac

SEQ ID NO: 38 - DNA, 20 bp, Artificial Sequence
  Example primer sequence, NJ-283, Hs-RPP30_QR
tttgtggtgatttcccccta

SEQ ID NO: 39 - DNA, 20 bp, Artificial Sequence
  Example primer sequence, NJ-284, Hs-ADM_QF
ggtcggactctggtgtcttc

SEQ ID NO: 40 - DNA, 20 bp, Artificial Sequence
  Example primer sequence, NJ-285, Hs-ADM_QR
cttgcgcgactattccttgt

SEQ ID NO: 41 - DNA, 20 bp, Artificial Sequence
  Example primer sequence, NJ-286, Hs-CFB_QF
caagcagacaagcaaagcaa

SEQ ID NO: 42 - DNA, 20 bp, Artificial Sequence
  Example primer sequence, NJ-287, Hs-CFB_QR
gataaagggcatcaggcaga

SEQ ID NO: 43 - DNA, 20 bp, Artificial Sequence
  Example primer sequence, NJ-288, Hs-Kiss1_QF
acctgccgaactacaactgg

SEQ ID NO: 44 - DNA, 21 bp, Artificial Sequence
  Example primer sequence, NJ-289, Hs-Kiss1_QR
tttggggtctgaagttcactg

SEQ ID NO: 45 - DNA, 19 bp, Artificial Sequence
  Example primer sequence, NJ-292, Hs-NCOA6_QF
tggcttctcagcaggacag

SEQ ID NO: 46 - DNA, 20 bp, Artificial Sequence
  Example primer sequence, NJ-293, Hs-NCOA6_QR
tgctggacattttgatttgc

SEQ ID NO: 47 - DNA, 20 bp, Artificial Sequence
  Example primer sequence, NJ-294, Hs-ADAM12_QF
cagttgcagcaggaaggact

SEQ ID NO: 48 - DNA, 20 bp, Artificial Sequence
  Example primer sequence, NJ-295, Hs-ADAM12_QR
tccacaaatctgttcccaca

SEQ ID NO: 49 - DNA, 78 bp, Artificial Sequence
  Example primer sequence, NJ-364, PE2_GRAMC_P4s
caagcagaagacggcatacgagatgtgactggagttcagacgtgtgctcttccgatctacagtccgacgatccagcag

SEQ ID NO: 50 - DNA, 75 bp, Artificial Sequence
  Example primer sequence, NJ-399, PE2_GRAMC_P3s
caagcagaagacggcatacgagatgtgactggagttcagacgtgtgctcttccgatcttagactccctcagcggc

SEQ ID NO: 51 - DNA, 75 bp, Artificial Sequence
  Example primer sequence, NJ-400, PE1_GRAMC_P3s
aatgatacggcgaccaccgagatctacactctttccctacacgacgctcttccgatcttagactccctcagcggc

SEQ ID NO: 52 - DNA, 75 bp, Artificial Sequence
  Example primer sequence, NJ-401, PE2_GRAMC_P2s
caagcagaagacggcatacgagatgtgactggagttcagacgtgtgctcttccgatctacactactctccagcag

SEQ ID NO: 53 - DNA, 79 bp, Artificial Sequence
  Example primer sequence, NJ-402, PE1_GRAMC_P4s
aatgatacggcgaccaccgagatctacactctttccctacacgacgctcttccgatcttacagtccgacgatccagcag

SEQ ID NO: 54 - DNA, 77 bp, Artificial Sequence
  Example primer sequence, NJ-403, PE2_GRAMC_P1s
caagcagaagacggcatacgagatgtgactggagttcagacgtgtgctcttccgatctttcactagtgattcagcag

SEQ ID NO: 55 - DNA, 20 bp, Artificial Sequence
  Example primer sequence, NJ-404, EGFPC1_QF1
aagggcatcgacttcaagga

SEQ ID NO: 56 - DNA, 20 bp, Artificial Sequence
  Example primer sequence, NJ-405, EGFPC1_QR1
ggcggatcttgaagttcacc

SEQ ID NO: 57 - DNA, 20 bp, Artificial Sequence
  Example primer sequence, NJ-443, GRAMc_GFP_QF2
gccctgtctaaagatcccaa

SEQ ID NO: 58 - DNA, 20 bp, Artificial Sequence
  Example primer sequence, NJ-444, GRAMc_GFP_QR2
cttgtacagctcgtccatgc

SEQ ID NO: 59 - DNA, 16 bp, Artificial Sequence
  Example primer sequence, NJ-489, GRAMc_RT_oligo
tacagtccgacgatcc

SEQ ID NO: 60 - DNA, 58 bp, Artificial Sequence
  Example primer sequence, NJ-497, PE1_adapter
aatgatacggcgaccaccgagatctacactctttccctacacgacgctcttccgatct

SEQ ID NO: 61 - DNA, 44 bp, Artificial Sequence
  Example primer sequence, NJ-498, PE1s_GRAMc_2N_P4s
  misc_feature (22)..(23): n is a, c, g, t or u
tacacgacgctcttccgatctnntacagtccgacgatccagcag

SEQ ID NO: 62 - DNA, 46 bp, Artificial Sequence
  Example primer sequence, NJ-499, PE1s_GRAMc_4N_P4s
  misc_feature (22)..(25): n is a, c, g, t or u
tacacgacgctcttccgatctnnnntacagtccgacgatccagcag

SEQ ID NO: 63 - DNA, 48 bp, Artificial Sequence
  Example primer sequence, NJ-500, PE1s_GRAMc_6N_P4s
  misc_feature (22)..(27): n is a, c, g, t or u
tacacgacgctcttccgatctnnnnnntacagtccgacgatccagcag

SEQ ID NO: 64 - DNA, 50 bp, Artificial Sequence
  Example primer sequence, NJ-501, PE1s_GRAMc_8N_P4s
  misc_feature (22)..(29): n is a, c, g, t or u
tacacgacgctcttccgatctnnnnnnnntacagtccgacgatccagcag

SEQ ID NO: 65 - DNA, 52 bp, Artificial Sequence
  Example primer sequence, NJ-502, PE1s_GRAMc_10N_P4s
  misc_feature (22)..(31): n is a, c, g, t or u
tacacgacgctcttccgatctnnnnnnnnnntacagtccgacgatccagcag

SEQ ID NO: 66 - DNA, 54 bp, Artificial Sequence
  Example primer sequence, NJ-503, PE1s_GRAMc_12N_P4s
  misc_feature (22)..(33): n is a, c, g, t or u
tacacgacgctcttccgatctnnnnnnnnnnnntacagtccgacgatccagcag

SEQ ID NO: 67 - DNA, 40 bp, Artificial Sequence
  Example primer sequence, NJ-504, PE1s_GRAMc_2N_nP3s
  misc_feature (22)..(23): n is a, c, g, t or u
tacacgacgctcttccgatctnntagactccctcagcggc

SEQ ID NO: 68 - DNA, 42 bp, Artificial Sequence
  Example primer sequence, NJ-505, PE1s_GRAMc_4N_nP3s
  misc_feature (22)..(25): n is a, c, g, t or u
tacacgacgctcttccgatctnnnntagactccctcagcggc

SEQ ID NO: 69 - DNA, 44 bp, Artificial Sequence
  Example primer sequence, NJ-506, PE1s_GRAMc_6N_nP3s
  misc_feature (22)..(27): n is a, c, g, t or u
tacacgacgctcttccgatctnnnnnntagactccctcagcggc

SEQ ID NO: 70 - DNA, 46 bp, Artificial Sequence
  Example primer sequence, NJ-507, PE1s_GRAMc_8N_nP3s
  misc_feature (22)..(29): n is a, c, g, t or u
tacacgacgctcttccgatctnnnnnnnntagactccctcagcggc

SEQ ID NO: 71 - DNA, 48 bp, Artificial Sequence
  Example primer sequence, NJ-508, PE1s_GRAMc_10N_nP3s
  misc_feature (22)..(31): n is a, c, g, t or u
tacacgacgctcttccgatctnnnnnnnnnntagactccctcagcggc

SEQ ID NO: 72 - DNA, 50 bp, Artificial Sequence
  Example primer sequence, NJ-509, PE1s_GRAMc_12N_nP3s
  misc_feature (22)..(33): n is a, c, g, t or u
tacacgacgctcttccgatctnnnnnnnnnnnntagactccctcagcggc

SEQ ID NO: 73 - DNA, 40 bp, Artificial Sequence
  Example primer sequence, NJ-523, GRAMc_Ion-P_nP3s
cctctctatgggcagtcggtgattagactccctcagcggc

SEQ ID NO: 74 - DNA, 44 bp, Artificial Sequence
  Example primer sequence, NJ-575, GRAMc_test1_F
ttcactagtgattcagcaggagtgccatcatgattcataaatag

SEQ ID NO: 75 - DNA, 44 bp, Artificial Sequence
  Example primer sequence, NJ-576, GRAMc_test1_R
acactactctccagcaggtacttaatatttgaggttactcgtag

SEQ ID NO: 76 - DNA, 37 bp, Artificial Sequence
  Example primer sequence, NJ-577, GRAMc_test2_F
ttcactagtgattcagcagcacctgaccactagtggg

SEQ ID NO: 77 - DNA, 40 bp, Artificial Sequence
  Example primer sequence, NJ-578, GRAMc_test2_R
acactactctccagcagcactttggaatccaaatttccag

SEQ ID NO: 78 - DNA, 40 bp, Artificial Sequence
  Example primer sequence, NJ-579, GRAMc_test3_F
ttcactagtgattcagcagcaagtacagcattgactgagc

SEQ ID NO: 79 - DNA, 36 bp, Artificial Sequence
  Example primer sequence, NJ-580, GRAMc_test3_R
acactactctccagcagagacagagctgacacacac

SEQ ID NO: 80 - DNA, 40 bp, Artificial Sequence
  Example primer sequence, NJ-589, GRAMc_test8_F
ttcactagtgattcagcagttattttgcttacagggccag

SEQ ID NO: 81 - DNA, 46 bp, Artificial Sequence
  Example primer sequence, NJ-590, GRAMc_test8_R
acactactctccagcaggtgacacaggagcttatatatatataagc

SEQ ID NO: 82 - DNA, 43 bp, Artificial Sequence
  Example primer sequence, NJ-591, GRAMc_test9_F
ttcactagtgattcagcagtacaatccacctacttaaagtgtg

SEQ ID NO: 83 - DNA, 39 bp, Artificial Sequence
  Example primer sequence, NJ-592, GRAMc_test9_R
acactactctccagcagttaaatagagacggggtttcac

SEQ ID NO: 84 - DNA, 43 bp, Artificial Sequence
  Example primer sequence, NJ-691, G5_1_F
ttcactagtgattcagcagcctttctaacttgggtcatttctg

SEQ ID NO: 85 - DNA, 41 bp, Artificial Sequence
  Example primer sequence, NJ-692, G5_1_R
acactactctccagcagctttctttatctacagcaaacagg

SEQ ID NO: 86 - DNA, 45 bp, Artificial Sequence
  Example primer sequence, NJ-693, G5_2_F
ttcactagtgattcagcagcacaagatacatgtagctgaatttag

SEQ ID NO: 87 - DNA, 43 bp, Artificial Sequence
  Example primer sequence, NJ-694, G5_2_R
acactactctccagcagtatttttagtagagacggggtttcac

SEQ ID NO: 88 - DNA, 40 bp, Artificial Sequence
  Example primer sequence, NJ-695, G5_3_F
ttcactagtgattcagcagaaaccctctaggtcctttaac

SEQ ID NO: 89 - DNA, 37 bp, Artificial Sequence
  Example primer sequence, NJ-696, G5_3_R
acactactctccagcagggattacaggaatgtgccac

SEQ ID NO: 90 - DNA, 39 bp, Artificial Sequence
  Example primer sequence, NJ-697, G5_4_F
ttcactagtgattcagcagaaaacaccacgtagtttggc

SEQ ID NO: 91 - DNA, 37 bp, Artificial Sequence
  Example primer sequence, NJ-699, G5_5_F
ttcactagtgattcagcagaagccagcgttgcccatc

SEQ ID NO: 92 - DNA, 36 bp, Artificial Sequence
  Example primer sequence, NJ-700, G5_5_R
acactactctccagcaggcctcagcctcctgagtag

SEQ ID NO: 93 - DNA, 39 bp, Artificial Sequence
  Example primer sequence, NJ-701, G5_6_F
ttcactagtgattcagcaggtaaatccaatcccaggttg

SEQ ID NO: 94 - DNA, 39 bp, Artificial Sequence
  Example primer sequence, NJ-702, G5_6_R
acactactctccagcaggccaccatgtttggctattttc

SEQ ID NO: 95 - DNA, 43 bp, Artificial Sequence
  Example primer sequence, NJ-705, G3_1_F
ttcactagtgattcagcagagttttggtattttaatactcttg

SEQ ID NO: 96 - DNA, 38 bp, Artificial Sequence
  Example primer sequence, NJ-706, G3_1_R
acactactctccagcagcattggttaagtgtagcaaac

SEQ ID NO: 97 - DNA, 43 bp, Artificial Sequence
  Example primer sequence, NJ-707, G3_2_F
ttcactagtgattcagcagatcatttttctttccgagatgttg

SEQ ID NO: 98 - DNA, 42 bp, Artificial Sequence
  Example primer sequence, NJ-708, G3_2_R
acactactctccagcagtattttttttgagatggagtttcgc

SEQ ID NO: 99 - DNA, 40 bp, Artificial Sequence
  Example primer sequence, NJ-709, G3_3_F
ttcactagtgattcagcagcccgttccacaaggatctgtg

SEQ ID NO: 100 - DNA, 38 bp, Artificial Sequence
  Example primer sequence, NJ-710, G3_3_R
acactactctccagcagctccggaatagctgggattac

SEQ ID NO: 101 - DNA, 45 bp, Artificial Sequence
  Example primer sequence, NJ-711, G3_4_F
ttcactagtgattcagcagtctccttataaatatctttcacttcc

SEQ ID NO: 102 - DNA, 38 bp, Artificial Sequence
  Example primer sequence, NJ-712, G3_4_R
acactactctccagcagagaattaagggggaaaagttg

SEQ ID NO: 103 - DNA, 37 bp, Artificial Sequence
  Example primer sequence, NJ-713, G3_5_F
ttcactagtgattcagcaggtggaatctggaggccag

SEQ ID NO: 104 - DNA, 40 bp, Artificial Sequence
  Example primer sequence, NJ-714, G3_5_R
acactactctccagcagttgttggctctggtttttctttg

SEQ ID NO: 105 - DNA, 41 bp, Artificial Sequence
  Example primer sequence, NJ-717, L1_1_F
ttcactagtgattcagcagcttccttcctaccttctttttc

SEQ ID NO: 106 - DNA, 37 bp, Artificial Sequence
  Example primer sequence, NJ-718, L1_1_R
acactactctccagcagaaaacctgggagtcccaaag

SEQ ID NO: 107 - DNA, 41 bp, Artificial Sequence
  Example primer sequence, NJ-719, L1_2_F
ttcactagtgattcagcagaccttcttacttcttaaggggg

SEQ ID NO: 108 - DNA, 40 bp, Artificial Sequence
  Example primer sequence, NJ-720, L1_2_R
acactactctccagcagtctgcgagtcctcctcttctttg

SEQ ID NO: 109 - DNA, 41 bp, Artificial Sequence
  Example primer sequence, NJ-723, L1_4_F
ttcactagtgattcagcaggcaaccagcttggaaatttctc

SEQ ID NO: 110 - DNA, 38 bp, Artificial Sequence
  Example primer sequence, NJ-724, L1_4_R
acactactctccagcagagacttcgacttcttcggatg

SEQ ID NO: 111 - DNA, 41 bp, Artificial Sequence
  Example primer sequence, NJ-727, L1_6_F
ttcactagtgattcagcagaactaacatggctgatgccttg

SEQ ID NO: 112 - DNA, 45 bp, Artificial Sequence
  Example primer sequence, NJ-728, L1_6_R
acactactctccagcagtatttggtttgcttagagtcctcctctg

SEQ ID NO: 113 - DNA, 18 bp, Artificial Sequence
  Example primer sequence, NJ-729, EGFP_5p_F
atggtgagcaagggcgag

SEQ ID NO: 114 - DNA, 20 bp, Artificial Sequence
  Example primer sequence, NJ-730, EGFP_3p_R
ttatctagatccggtggatc

SEQ ID NO: 115 - DNA, 44 bp, Artificial Sequence
  Example primer sequence, NJ-731, EGFP_GRAMc_gibson_F
gatccaccggatctagataagcctctagactccctcagcggcgc

SEQ ID NO: 116 - DNA, 42 bp, Artificial Sequence
  Example primer sequence, NJ-732, EGFP_GRAMc_gibson_R
ctcgcccttgctcaccatttgtgattcacttgtaagatgacg

SEQ ID NO: 117 - DNA, 17 bp, Artificial Sequence
  Example trimming adaptor sequence, GRAMcP1s-SE.fa
tcactagtgattcagca

SEQ ID NO: 118 - DNA, 16 bp, Artificial Sequence
  Example trimming adaptor sequence, GRAMcP2s-SE.fa
acactactctccagca

SEQ ID NO: 119 - DNA, 18 bp, Artificial Sequence
  Example trimming adaptor sequence, GRAMcP3s-SE.fa
actccctcagcggcgcgc

SEQ ID NO: 120 - DNA, 17 bp, Artificial Sequence
  Example trimming adaptor sequence, GRAMcP4s-SE.fa
agtccgacgatccagca

SEQ ID NO: 121 - DNA, 18 bp, Artificial Sequence
  Example trimming adaptor sequence, GRAMcP1sr-SE.fa
ctgctgaatcactagtga

SEQ ID NO: 122 - DNA, 17 bp, Artificial Sequence
  Example trimming adaptor sequence, GRAMcP2sr-SE.fa
ctgctggagagtagtgt

SEQ ID NO: 123 - DNA, 18 bp, Artificial Sequence
  Example trimming adaptor sequence, GRAMcP3sr-SE.fa
gcgcgccgctgagggagt

SEQ ID NO: 124 - DNA, 16 bp, Artificial Sequence
  Example trimming adaptor sequence, GRAMcP4sr-SE.fa
tgctggatcgtcggac